(19) United States
(12) Patent Application Publication
(10) Pub. No.: US 2024/0228480 A1
(43) Pub. Date: Jul. 11, 2024

(54) PROCESSES FOR PREPARING PYRROLOPYRIDINE-ANILINE COMPOUNDS

(71) Applicant: NFLECTION THERAPEUTICS, INC., Boston, MA (US)

(72) Inventors: Michael HOUGHTON, Boston, MA (US); John KINCAID, Boston, MA (US); Ananya SENGUPTA, Boston, MA (US)

(21) Appl. No.: 18/273,322

(22) PCT Filed: Jan. 20, 2022
(86) PCT No.: PCT/US2022/013153

§ 371 Date: Jul. 20, 2023

Related U.S. Application Data
(60) Provisional application No. 63/139,981, filed on Jan. 21, 2021.

(51) Int. Cl. C07D 471/04; C07C 213/02; C07C 309/49

(52) U.S. Cl. CPC C07D 471/04; C07C 213/02; C07C 309/49

(57) ABSTRACT

The present disclosure provides processes for preparing a compound of formula (I) from a compound of formula (II) via two steps: 6a) contacting 2-(aminooxy)ethanol (i.e., formula (K)) or a salt thereof (e.g., formula (K-1)), with abase and a silylating agent to form a first mixture including an O-silyl protected compound of formula (K); and 6b) adding a second mixture including a compound of formula (II) or a salt therefore, to the first mixture of step 6a) to form the compound represented by formula (I): The present processes only utilize less than 1.5 equivalents of 2-(aminooxy)ethanol or the salt thereof relative to the compound of formula (II), and therefore reduce the burden to remove excess 2-(aminooxy)ethanol on a large manufacturing scale. Also provided are processes for preparing the compound of formula (K) or (K-1).

### CROSS-REFERENCES TO RELATED APPLICATIONS

This application claims priority to U.S. Provisional Application No. 63/139,981 filed Jan. 21, 2021, which is incorporated in its entity for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE DISCLOSURE

Neurofibromatosis type 1 (NF1) occurs in approximately 1:3,500 births, and is one of the most common autosomal dominant single-gene disorders affecting neurological function in humans. Clinically, NF1 disease is characterized by the presence of benign peripheral nerve tumors, called neurofibromas, involving Schwann cells with biallelic mutations in the NF1 gene, as well as other tumor and non-tumor manifestations. See Jousma et al. Pediatr. Blood Cancer 62: 1709-1716, 2015. NF1 is associated with several dermal disorders, including dermal neurofibromas; plexiform neurofibromas; café au lait spots; and axillary and inguinal freckling. Dermal neurofibromas occur in over 95% of NF1 patients, and can appear anywhere on the body, causing itching, irritation, infection, physical pain, and disfigurement. Moreover, dermal neurofibromas are associated with social isolation and anxiety.

NF1 is caused by one or more germ line mutations in NF1, a gene that inactivates the RAS pathway. Because the NF1 gene encodes a Ras-GAP protein, NF1 loss results in high Ras-GTP. Therefore, NF1 research has focused intensively on testing inhibitors in the Ras signaling pathway, including the Ras-MAPK cascade. See Jousma et al. Pediatr. Blood Cancer 62: 1709-1716, 2015. Four distinct MAPK cascades have been identified and named according to their MAPK module. See Akinleye et al. Journal of Hematology & Oncology 6:27, 2013. MEK proteins belong to a family of enzymes that lie upstream to their specific MAPK targets in each of the four MAP kinase signaling pathways. Two of these MEK proteins, MEK1 and MEK2, are closely related and participate in this signaling pathway cascade. Inhibitors of MEK1 and MEK2 have been shown to effectively inhibit MEK signaling downstream of Ras, and thus provide a strong rationale for targeting MEK in the treatment of NF1. See Rice et al. Medicinal Chemistry Letters 3:416-421, 2012.

Currently available MEK inhibitors are designed to have oral bioavailability for systemic delivery, and are associated with significant side effects including decreased left ventricular ejection fraction, elevated creatine phosphokinase, pneumonitis, renal failure, diarrhea, infection, uticaria, and maculo-papular rash, all of which are dose limiting or require permanent discontinuation. Moreover, clinical trials have shown side effects with prolonged high-dose administration of MEK inhibitors. See Huang et al. J. Ocul. Pharmacol. Ther. 25:519-530, 2009. For example, PD0325901, a MEK inhibitor currently in clinical trials, has exhibited neurological side effects associated with ataxia, confusion, and syncope. In addition, a number of other side effects have been observed with systemic exposure to MEK inhibitors including: acneiform rash, CPK elevation, nausea, vomiting, diarrhea, abdominal pain, and fatigue. Thus, there is a need for therapies that inhibit MEK to treat NF1 associated dermal neurofibromas, which limit these serious side effects.

Benign cutaneous tumors of the vascular, keratinocytic, and melanocytic compartments often occur at birth or during childhood. These lesions, referred in this application as “birthmarks”, can cause cosmetic distress, disfigurement and social anxiety. In some cases, these lesions can predispose individuals to functional impairment or future malignancies. These birthmarks can be sporadic or arise as part of an underlying neurocutaneous syndrome.

Vascular birthmarks include, for example port wine stain/capillary malformation, angiomas, lobular capillary hemangiomas, arteriovascular malformation, lymphatic malformation, vascular malformation, hemangiomas, and other angioma. Keratinocytic nevi refers to Keratinocytic epidermal nevi and nevi sebacei. Melanocytic nevi (commonly known as moles) include, for example congenital nevi, multiple lentigines (which can occur in syndromes such as LEOPARD), ephiledes (freckles), and nevus spiilus.

Neurocutaneous syndromes, also referred to as birthmarks, such as port-wine stains, are associated with congenital low-flow vascular malformations (capillary malformation) in the skin which, if left untreated, can hypertrophy and develop nodularity (Minkis, K. et al, Lasers Surg Med. (2009) 41(6): pp 423-426). Laser therapy is typically used for treatment of port-wine stains, but often without full resolution. Epidermal nevi are common cutaneous mosaic disorders, subdivided into keratinocytic and organoid nevi. Organoid nevi include nevus sebaceus (NS). Immunolabelling of NS is reportedly associated with increased phosphorylated ERK staining (Aslam, A, et al., Clinical and ExperimentalDermatology (2014) 39: pp 1-6). Non-organoid keratinocytic epidermal nevus (KEN) is characterized by benign congenital hyperpigmented skin lesions. Epidermal nevi with localized epidermal thickening are present at birth or become visible during childhood. Other cutaneous disorders that also occur in childhood birthmarks include nevus cellular nevus, lobulary capillary hemangioma, congenital nevi, ephiledes (freckles), multiple lentigines (which can occur in multiple syndromes including LEOPARD syndrome), capillary angioma, nevus spilus, arterio-venous malformations, lymphatic malformations, and congenital melanocytic nevus. Lentigines can occur in childhood (in syndromes such as LEOPARD syndrome), which has mutations that activate RAS/MAPK pathway, as well as can be acquired in adults. In some cases birthmarks are not amenable to surgical excision and/or laser treatment. In some cases birthmarks, when untreated, can progress to lesions and/or proliferative skin conditions.

Modulation of ERK/MEK pathways may have a therapeutic effect on birthmarks. RAS mutations have been reported in mosaic RASopathies i.e. non-organoid KEN, and sebaceous nevus (Farschtschi S, et al., BMC Medical Genetics. (2015); 16: pp 6; and Sun, B. K. et. Al, Journal of Investigative Dermatology, (2013); 3: pp 824-827). Thus, inhibition of Ras signaling pathway, including the Ras-MAPK cascade, may be useful in treating birthmarks.

Four distinct MAPK cascades have been identified and named according to their MAPK module. See Akinleye et al. Journal of Hematology & Oncology 6:27, 2013. MEK proteins belong to a family of enzymes that lie upstream to their specific MAPK targets in each of the four MAP kinase signaling pathways. Two of these MEK proteins, MEK1 and MEK2, are closely related and participate in this signaling pathway cascade. Inhibitors of MEK1 and MEK2 have been shown to effectively inhibit MEK signaling downstream of Ras (Rice et al. Medicinal Chemistry Letters 3:416-421, 2012), and thus provide a rationale for targeting MEK in the treatment of birthmarks.

Currently available MEK pathway inhibitors are designed to have oral bioavailability for systemic delivery, but are associated with one or more significant side effects including decreased left ventricular ejection fraction, elevated creatine phosphokinase, pneumonitis, renal failure, diarrhea, infection, uticaria, and maculo-papular rash, all of which are dose limiting or require permanent discontinuation. Moreover, clinical trials have shown one or more side effects with prolonged high-dose administration of MEK inhibitors. (Huang et al. J. Ocul. Pharmacol. Ther. 25:519-530, 2009). For example, PD0325901, a clinically-tested MEK inhibitor, has exhibited one or more neurological side effects associated with ataxia, confusion, and syncope. In addition, a number of other side effects have been observed with systemic exposure to MEK inhibitors including: acneiform rash, CPK elevation, nausea, vomiting, diarrhea, abdominal pain, and fatigue. Thus, there is a need for therapies that treat birthmarks and also limit one or more side effects associated with systemic exposure to MEK/ERK pathway inhibitors.

A compound of formula (I) was first disclosed in WO 2018/213810 as a MEK inhibitor for the treatment of dermal diseases or dermal disorders associated therewith. As described in WO 2018/213810, the compound of formula (I) was prepared by reacting a compound represented by formula (II):

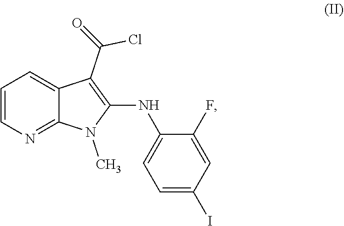 [SMILES: Cn1c(Nc2ccc(I)cc2F)c(C(=O)Cl)c2cccnc21]

or a salt therefore, with 5 equivalents of 2-(aminooxy)ethanol in THF. The disclosed reaction requires a large excess of 2-(aminooxy)ethanol, which poses a challenge to remove on a large manufacturing scale. More importantly, the reaction is very sensitive to impurities (e.g., ethylene glycol, certain solvent residues such as DMSO, DMF, et. al.) present in the material of 2-(aminooxy)ethanol, therefore a rigid specification is required to meet in order to ensure the successful manufacturing of the compound of formula (I) as an active ingredient (API). However, commercial available 2-(aminooxy)ethanol provides inconsistent purity and impurity profiles of the material, which in turn can bring a severe impact on the quality of the final product as an active ingredient (API). Therefore, there remains a need to development improved processes suitable for manufacturing the compound of formula (I) on a large scale. The present disclosure addresses this need and provides related advantages as well.

### BRIEF SUMMARY OF THE DISCLOSURE

In a first aspect, the present disclosure provides a process for preparing a compound represented by formula (I):

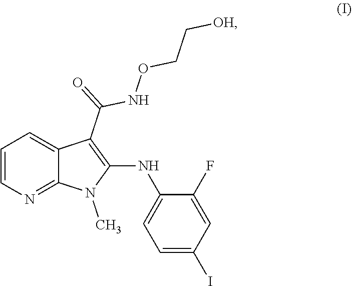

or a salt thereof, the process including:
6a) contacting a compound represented by formula (K):

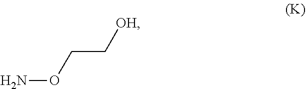

or a salt thereof, with a first base and a silylating agent in a first solvent to form a first mixture comprising an O-silyl protected compound of formula (K); and
6b) adding a second mixture comprising a compound represented by formula (II):

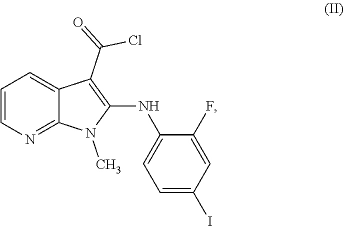

or a salt therefore, to the first mixture of step 6a) to form the compound represented by formula (I).

In a second aspect, the present disclosure provides a process for preparing a compound represented by formula (I):

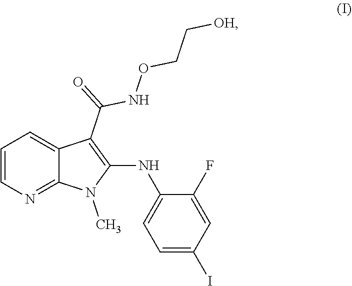

or a salt thereof, the process including:
3) converting a compound represented by formula (VI):

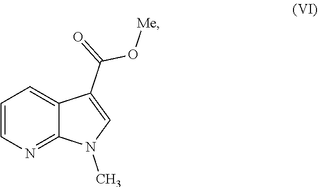

or a salt thereof, to a compound represented by formula (V):

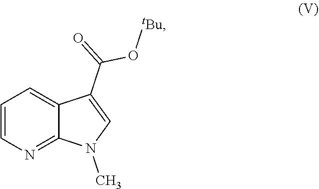

or a salt thereof, with sodium tert-butoxide in toluene;
4a) contacting the compound represented by formula (V) or the salt thereof with hexachloroethane and lithium bis(trimethylsilyl)amide (LiHMDS) in THF to form a compound represented by formula (IVa):

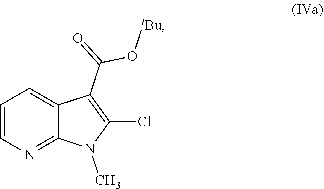

or a salt thereof;
4b) adding an aniline represented by formula (L):

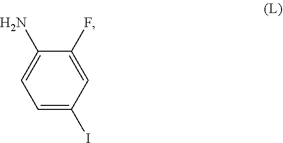

to a reaction mixture of step 4a) comprising the compound of formula (IVa) or the salt
thereof to form a compound represented by formula (III):

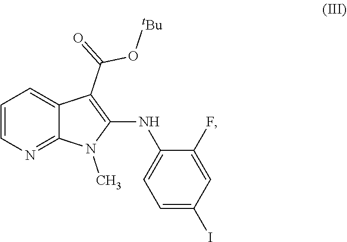

or a salt thereof;
5) contacting the compound represented by formula (III) or the salt thereof with thionyl chloride and hydrogen chloride in a 1,4-dioxane to form a HCl salt of a compound represented by formula (II):

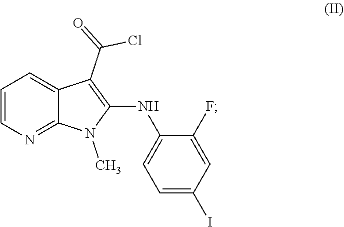

6a) contacting a compound represented by formula (K) or a p-toluenesulfonic acid salt thereof represented by formula (K-1):

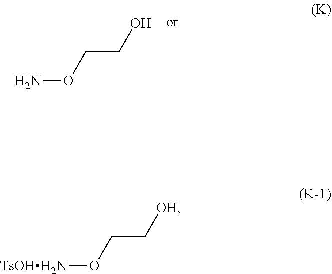

with 4-methylmorpholine and trimethylsilyl chloride (TMSCl) in tetrahydrofuran (THF) or methyl tert-butyl ether (MTBE) to form a first mixture; and
6b) adding a second mixture comprising the HCl salt of formula (II), and tetrahydrofuran (THF) or methyl tert-butyl ether (MTBE), to the first mixture of step 6a) to form the compound represented by formula (I) or the salt thereof.

In a third aspect, the present disclosure provides a process for preparing a compound represented by formula (K):

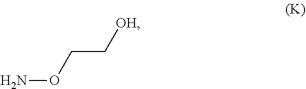

or a salt thereof, the process including:
7) contacting 2-hydroxyisoindoline-1,3-dione represented by the formula:

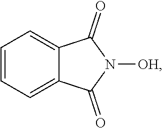 [SMILES: O=C1c2ccccc2C(=O)N1O]

with 2-bromoethanol and a non-nucleophilic base in an aprotic solvent to form
2-(2-hydroxyethoxy)isoindoline-1,3-dione represented by formula (J):

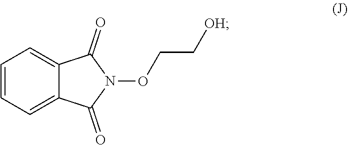 [SMILES: O=C1c2ccccc2C(=O)N1OCCO]

8a) treating 2-(2-hydroxyethoxy)isoindoline-1,3-dione with ammonia in an alcohol solvent to provide the compound of formula (K); and
8b) optionally converting the compound of formula (K) to the salt thereof.

In a fourth aspect, the present disclosure provides a process for preparing a MEK inhibitor represented by formula (XI):

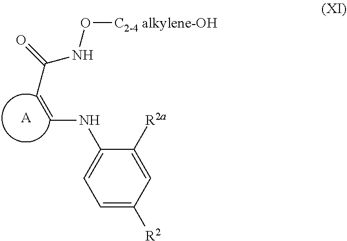

or a salt thereof, the process including:
a) contacting a compound of H₂N—O—C₂₋₄ alkylene-OH or a salt thereof, with a first base and a silylating agent in a first solvent to form a first mixture including an O-silyl protected compound thereof; and
b) reacting the first mixture with a compound represented by formula (XII):

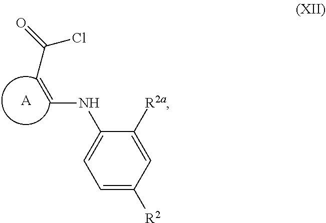

or a salt therefore to form the compound represented by formula (XI),
   wherein:
A ring is C₆₋₁₂ aryl or a 5-10 membered heteroaryl having 1 to 4 heteroatoms or groups as ring vertices independently selected from N, C(O), O, and S, each of which is unsubstituted or substituted; and
R² and R^{2a} are each independently halo, C₁₋₆ alkyl, —S—C₁₋₆ alkyl, C₂₋₆ alkenyl, or C₂₋₆ alkynyl.

In a fifth aspect, the present disclosure provides a process for preparing a MEK inhibitor represented by formula (XI):

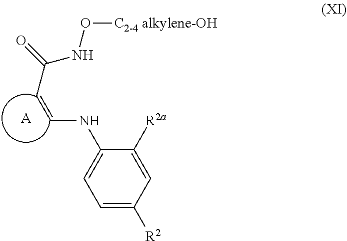

or a salt thereof, the process including:
a) contacting a compound of H₂N—O—C₂₋₄ alkylene-OH or a salt thereof, with a first base and a silylating agent in a first solvent to form a first mixture including an O-silyl protected compound thereof; and
b) reacting the first mixture with a compound represented by formula (XIII):

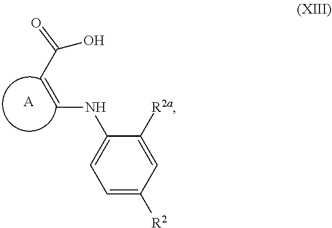

or a salt therefore to form the compound represented by formula (XI),
   wherein:
A ring is C₆₋₁₂ aryl or a 5-10 membered heteroaryl having 1 to 4 heteroatoms or groups as ring vertices independently selected from N, C(O), O, and S, each of which is unsubstituted or substituted; and
R² and R^{2a} are each independently halo, C₁₋₆ alkyl, —S—C₁₋₆ alkyl, C₂₋₆ alkenyl, or C₂₋₆ alkynyl.

In a sixth aspect, the present disclosure provides a compound represented by formula (X):

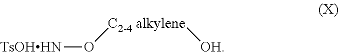

### BRIEF DESCRIPTION OF THE DRAWINGS

FIG. 1 shows one of embodiments for preparing a compound of formula (I).

FIG. 2 shows one of embodiments for preparing 2-(aminooxy)ethanol (i.e., formula (K) and/or a p-toluenesulfonic acid salt of 2-(aminooxy)ethanol (i.e., formula (K-1)).

FIG. 3 shows selected embodiments for preparing a compound of formula (I), via steps 4-6.

### DETAILED DESCRIPTION OF THE DISCLOSURE

### I. General

The present disclosure provides processes for preparing a compound of formula (I) from a compound of formula (II) via two steps: 6a) contacting 2-(aminooxy)ethanol (i.e., formula (K)) or a salt thereof (e.g., formula (K-1)), with a base and a silylating agent to form a first mixture including an O-silyl protected compound of formula (K); and 6b) adding a second mixture including a compound of formula (II) or a salt therefore, to the first mixture of step 6a) to form the compound represented by formula (I). The present processes only utilize less than 1.5 equivalents of 2-(aminooxy)ethanol or the salt thereof relative to the compound of formula (II), and therefore reduce the burden to remove excess 2-(aminooxy)ethanol on a large manufacturing scale. The present process has provided the compound of formula (I) as an active ingredient (API) on a large manufacturing scale of about 5 kilograms with a purity and impurity profile meeting requirements for pharmaceutical development.

In order to have consistent purity and/or impurity profile of 2-(aminooxy)ethanol, the present disclosure also provides processes for preparing 2-(aminooxy)ethanol or a salt thereof, in particular a p-toluenesulfonic acid salt thereof. Surprisingly, when p-toluenesulfonic acid salt of 2-(aminooxy)ethanol (i.e., formula (K-1)) is used in step 6a), the conversion of the compound of formula (II) to the compound of formula (I) proceeds unexpectedly well. As a result, the compound of formula (I) can be isolated in a high purity of >95 area % by HPLC or UPLC method.

### II. Definitions

“Alkyl” refers to a straight or branched, saturated, aliphatic radical having the number of carbon atoms indicated (i.e., C₁₋₆ means one to six carbons). Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄-5, C₄₋₆ and C₅-6. For example, C₁₋₆ alkyl includes, but is not limited to, methyl, ethyl, propyl, isopropyl, butyl, isobutyl, sec-butyl, tert-butyl, pentyl, isopentyl, hexyl, etc.

“Alkylene” refers to a straight or branched, saturated, aliphatic radical having the number of carbon atoms indicated (i.e., C₁₋₆ means one to six carbons), and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene.

“Alkenyl” refers to a straight chain or branched hydrocarbon having at least 2 carbon atoms and at least one double bond and having the number of carbon atom indicated (i.e., C₂₋₆ means to two to six carbons). Alkenyl can include any number of carbons, such as C₂, C₂₋₃, C₂₋₄, C₂₋₅, C₂₋₆, C₂₋₇, C₂-s, C₂₋₉, C₂₋₁₀, C₃, C₃0.4, C₃0.5, C₃₋₆, C₄, C₄0.5, C₄0.6, C₅, C₅0.6, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl.

“Alkynyl” refers to either a straight chain or branched hydrocarbon having at least 2 carbon atoms and at least one triple bond and having the number of carbon atom indicated (i.e., C₂₋₆ means to two to six carbons). Alkynyl can include any number of carbons, such as C₂, C₂₋₃, C₂₋₄, C₂₋₅, C₂₋₆, C₂₋₇, C₂-s, C₂₋₉, C₂₋₁₀, C₃, C₃0.4, C₃0.5, C₃₋₆, C₄, C₄0.5, C₄0.6, C₅, C₅0.6, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl.

“Halogen” refers to fluorine, chlorine, bromine and iodine.

“Alkoxy” refers to an alkyl group having an oxygen atom that connects the alkyl group to the point of attachment: alkyl-O—. Alkoxy groups can have any suitable number of carbon atoms, such as C₁-C₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. The heteroatoms can also be oxidized, such as, but not limited to, —S(O)— and —S(O)₂—. Heteroaryl groups can include any number of ring atoms, such as, 5 to 6, 5 to 8, 6 to 8, 5 to 9, 5 to 10, 5 to 11, or 5 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 10 ring members and from 1 to 4 heteroatoms, from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Silylating agent” refers to an agent that can introduce a silyl group (R₃Si) to a molecule, wherein the R groups can be alkyl. Non-limiting examples of silylating agents include tert-butyldimethylsilyl chloride, triethylsilyl chloride, and trimethyl chloride.

“Base” refers to a functional group that deprotonates water to produce a hydroxide ion. Bases useful in the present disclosure include organic bases and inorganic bases. Exemplary organic bases include amines, alkali carboxylates, alkali alkoxides, metal amides, and alkyl or alkenyl-metal compounds, as defined herein. Exemplary inorganic bases include alkali bicarbonates, alkali carbonates, alkali phosphates tribasic, alkali phosphate dibasic, alkali hydroxides, and alkali hydride, as defined herein. Amines useful in the present disclosure as bases include tertiary amines, aromatic amine bases, and amidine-based compounds, as defined herein.

“First base”, “second base”, and so on refer to a base as defined above and described in embodiments of the present disclosure. The base naming conventions are used solely for the purpose of clarity in relevant steps of the process as described herein and they are not required to be in a numerical order. Some bases may be absent in selected embodiments of the present disclosure as described herein. One skilled in the art will understand the meaning of these base naming conventions (‘first base’, ‘second base’) within the context of the term's use in the embodiments and claims herein.

“Non-nucleophilic base” refers to a sterically hindered organic base that is a poor nucleophile. Non-limiting examples of non-nucleophilic bases include tertiary amines and amidine-based compounds as defined herein.

“Tertiary amine” refers to a compound having formula N(R)₃ wherein the R groups can be alkyl, aryl, heteroalkyl, heteroaryl, among others, or two R groups together form a N-linked heterocycloalkyl. The R groups can be the same or different. Non-limiting examples of tertiary amines include triethylamine, tri-n-butylamine, N,N-diisopropylethylamine, N-methylpyrrolidine, N-methylmorpholine, dimethylaniline, diethylaniline, 1,8-bis(dimethylamino)naphthalene, quinuclidine, and 1,4-diazabicylo[2.2.2]-octane (DABCO).

“Aromatic amine base” refers to a N-containing 5- to 10-membered heteroaryl compound or a tertiary amine having formula N(R)₃ wherein at least one R group is an aryl or heteroaryl. Aromatic amine bases useful in the present application include, but are not limited to, pyridine, lutidines (e.g., 2,6-lutidine, 3,5-lutidine, and 2,3-lutidine), collidines (e.g., 2,3,4-collidine, 2,3,5-collidine, 2,3,6-collidine, 2,4,5-collidine, 2,4,6-collidine, and 3,4,5-collidine), 4-dimethylaminopyridine, imidazole, dimethylaniline, and diethylaniline.

“Amidine-based compounds” herein refers to a class of chemical compounds that include, but are not limited to, 1,8-diazabicyclo[5.4.0]undec-7-ene (DBU) and 1,5-diazabicyclo[4.3.0]non-5-en (DBN).

“Alkali carboxylate” refers to a class of chemical compounds which are composed of an alkali metal cation or a phosphonium and the carboxylate anion (RC(O)O⁻) where the R group can be alkyl or aryl. Carboxylates useful in the present include, but are not limited to, lithium acetate (LiOC(O)CH₃), sodium acetate (NaOC(O)CH₃), potassium acetate (KOC(O)CH₃), cesium acetate (CsOC(O)CH₃), potassium trimethylacetate (KOC(O)C(CH₃)₃), and tetrabutylphosphonium malonate.

“Alkali bicarbonate” refers to a class of chemical compounds which are composed of an alkali metal cation and the hydrogencarbonate anion (HCO₃⁻). Alkali carbonates useful in the present disclosure include lithium bicarbonate (LiHCO₃), sodium bicarbonate (NaHCO₃), potassium bicarbonate (KHCO₃), and cesium bicarbonate (CsHCO₃).

“Alkali carbonate” refers to a class of chemical compounds which are composed of an alkali metal cation and the carbonate anion (CO₃²⁻). Alkali carbonates useful in the present disclosure include lithium carbonate (Li₂CO₃), sodium carbonate (Na₂CO₃), potassium carbonate (K₂CO₃), and cesium carbonate (Cs₂CO₃).

“Alkali phosphate tribasic” refers to a class of chemical compounds which are composed of an alkali metal cation and the phosphate anion (PO₄³⁻). Alkali phosphates tribasic useful in the present disclosure include sodium phosphate tribasic (Na₃PO₄) and potassium phosphate tribasic (K₃PO₄).

“Alkali phosphate dibasic” refers to a class of chemical compounds which are composed of an alkali metal cation and the hydrogenphosphate anion (HPO₄²⁻). Alkali phosphates dibasic useful in the present disclosure include sodium phosphate dibasic (Na₂HPO₄) and potassium phosphate dibasic (K₂HPO₄).

“Alkali hydroxide” refers to a class of chemical compounds which are composed of an alkali metal cation and the hydroxide anion (OH⁻). Alkali hydroxides useful in the present disclosure include lithium hydroxide (LiOH), sodium hydroxide (NaOH), potassium hydroxide (KOH), and cesium hydroxide (CsOH).

“Alkali alkoxide” refers to a class of chemical compounds which are composed of an alkali metal cation and the alkoxide anion (RO⁻), wherein R is C₁₋₄ alkyl. Alkali alkoxides useful in the present disclosure include, but are not limited to, sodium isopropoxide, sodium methoxide, sodium tert-butoxide, potassium tert-butoxide, and potassium isopropoxide.

“Metal amide” refers to a class of coordination compounds composed of a metal center with amide ligands of the form —NR₂, wherein R is alkyl, cycloalkyl, or silyl. Metal amides useful in the present disclosure include, but are not limited to, lithium diisopropylamide, lithium bis(trimethylsilyl)amide, potassium bis(trimethylsilyl)-amide, lithium 2,2,6,6,-tetramethylpiperidide, 2,2,6,6-tetramethylpiperidinylmagnesium chloride, bis(2,2,6,6-tetramethylpiperidinyl)magnesium, and di-n-butyllithium(2,2,6,6-tetramethylpiperidinyl)magnesate).

“Alkyl- and alkenylmetal compound” refers to a class of chemical compounds composed of a metal center bond to alkyl or alkenyl. Alkyl- and alkenylmetal compounds useful in the present disclosure include, but are not limited to, n-butyllithium, isopropylmagnesium chloride, tri-n-butyllithium magnesate, di-n-butylmagnesium, di-sec-butylmagnesium, and ethyl n-butylmagnesium.

“Alkali hydride” refers to a class of chemical compounds composed of an alkali metal cation and the hydride anion (H⁻). Alkali hydrides useful in the present disclosure include lithium hydride, sodium hydride and potassium hydride.

“Solvent” refers to a substance, such as a liquid, capable of dissolving a solute. Solvents can be polar or non-polar, protic or aprotic. Polar solvents typically have a dielectric constant greater than about 5 or a dipole moment above about 1.0, and non-polar solvents have a dielectric constant below about 5 or a dipole moment below about 1.0. Protic solvents are characterized by having a proton available for removal, such as by having a hydroxy or carboxy group. Aprotic solvents lack such a group. Representative polar protic solvents include alcohols (methanol, ethanol, propanol, isopropanol, etc.), acids (formic acid, acetic acid, etc.) and water. Representative polar aprotic solvents include dichloromethane, chloroform, tetrahydrofuran, methyltetrahydrofuran, diethyl ether, 1,4-dioxane, acetone, ethyl acetate, dimethylformamide, acetonitrile, dimethyl sulfoxide, and N-methylpyrrolidone. Representative non-polar solvents include alkanes (pentanes, hexanes, etc.), cycloalkanes (cyclopentane, cyclohexane, etc.), benzene, and toluene. Other solvents are useful in the present disclosure.

“Aprotic solvent” refers to solvents that lack an acidic hydrogen. Consequently, they are not hydrogen bond donors. Common characteristics of aprotic solvents are solvents that can accept hydrogen bonds, solvents do not have acidic hydrogen, and solvents dissolve salts. Examples of aprotic solvents include, but are not limited to, N-methylpyrrolidone (NMP), tetrahydrofuran (THF), 2-methyl tetrahydrofuran (MeTHF), ethyl acetate (EtOAc), acetone, dimethylformamide (DMF), acetonitrile (MeCN), dimethyl sulfoxide (DMSO), propylene carbonate (PC), and hexamethylphosphoramide (HMPA).

“First solvent”, “second solvent”, and so on refer to a solvent as defined above and described in embodiments of the present disclosure. The solvent naming conventions are used solely for the purpose of clarity in steps of the process as described herein and they are not required to be in a numerical order. Some solvents may be absent in selected embodiments of the present disclosure as described herein. One skilled in the art will understand the meaning of these solvent naming conventions (e.g., ‘first solvent’, ‘second solvent’) within the context of the term's use in the embodiments and claims herein.

“Chlorinating agent” refers to a reagent capable of adding a chloro group, —Cl, to a compound. Representative chlorinating agents include, but are not limited to, phosphorous oxychloride, thionyl chloride, oxalyl chloride and sulfuryl chloride.

“First chlorinating agent” and “second chlorinating agent” refer to a chlorinating agent as defined above and described in embodiments of the present disclosure. The chlorinating agent naming conventions are used solely for the purpose of clarity in steps of the process as described herein and they are not required to be in a numerical order. One skilled in the art will understand the meaning of these chlorinating agent naming conventions (e.g., ‘first chlorinating agent’, ‘second chlorinating agent’) within the context of the term's use in the embodiments and claims herein.

For clarity purpose, the Table below summarizes the solvent, base, and chlorinating agent naming conventions used in corresponding process steps 3) to 6):

| Process Step | Solvent | Base | chlorinating agent |
|---|---|---|---|
| 6a) | first solvent | first base | — |
| 6b), a | second solvent | — | — |
| second mixture | | | |
| 5) | third solvent | — | first |
| | | | chlorinating agent |
| 4a) | fourth solvent | second base | second |
| | | | chlorinating agent |
| 4b) | fifth solvent | third base | — |
| 3) | six solvent | — | — |

“Iodinating agent” refers to a reagent capable of adding an iodo group, —I, to a compound. Representative iodinating agents include, but are not limited to, iodine and N-iodo-bis(trimethylsily)amide.

“Protecting group” refers to a compound that renders a functional group unreactive to a particular set of reaction conditions, but that is then removable in a later synthetic step so as to restore the functional group to its original state. Such protecting groups are well known to one of ordinary skill in the art and include compounds that are disclosed in “Protective Groups in Organic Synthesis”, 4th edition, T. W. Greene and P. G. M. Wuts, John Wiley & Sons, New York, 2006, which is incorporated herein by reference in its entirety.

“Contacting” refers to the process of bringing into contact at least two distinct species such that they can react. It should be appreciated, however, the resulting reaction product can be produced directly from a reaction between the added reagents or from an intermediate from one or more of the added reagents which can be produced in the reaction mixture.

“Deprotecting” refers to remove the protecting group as defined above (e.g., the silyl group) using one or more chemicals or agents so that the functional group (—OH group) is restored to its original state.

“Crude” refers to a mixture including a desired compound (e.g., the compound of formula (I)) and at least one other species (e.g., a solvent, a reagent such as an acid or base, a starting material, or a byproduct of a reaction giving rise to the desired compound).

Unless specifically indicated otherwise, “purity %” or “purity area %” (e.g., 95% or 95 area %) refers to a purity of a compound (e.g., the compound of formula (I)) in the area under curve (AUC) determined by a HPLC or UPLC method (e.g., Chemical Development HPLC Method or UPLC method as described herein).

“Salt” refers to acid or base salts of the compounds used in the methods of the present disclosure. Salts useful in the present disclosure include, but are not limited to, phosphate, sulfate, chloride, bromide, carbonate, nitrate, acetate, methanesulfonate, sodium, potassium, and calcium salts. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts, and alkaline metal or alkaline earth metal salts (sodium, potassium, calcium, and the like). It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“A,” “an,” or “a(n)”, when used in reference to a group of substituents or “substituent group” herein, mean at least one. For example, where a compound is substituted with “an” alkyl or aryl, the compound is optionally substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Processes for Preparing a Compound of Formula (I)

In a first aspect, the present disclosure provides a process for preparing a compound represented by formula (I):

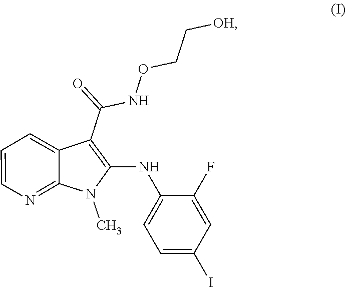 [SMILES: Cn1c(Nc2ccc(I)cc2F)c(C(=O)NOCCO)c2cccnc21]

or a salt thereof, the process including:
6a) contacting a compound represented by formula (K):

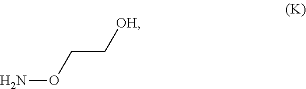

or a salt thereof, with a first base and a silylating agent in a first solvent to form a first mixture comprising an O-silyl protected compound of formula (K); and
6b) adding a second mixture comprising a compound represented by formula (II):

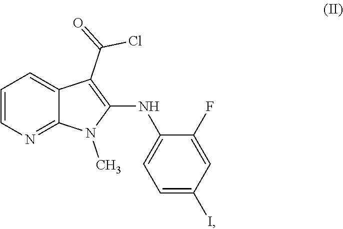

or a salt therefore, to the first mixture of step 6a) to form the compound represented by formula (I).

### A. Step 6

The compound of formula (K) can be in a neutral form or in a salt form. In some embodiments, the compound of formula (K) is in a neutral form. In some embodiments, the compound of formula (K) is a salt thereof. In some embodiments, the compound of formula (K) is a HCl, a sulfate, a hemisulfate, or a p-toluenesulfonic acid salt thereof. In some embodiments, the compound of formula (K) is a p-toluenesulfonic acid salt thereof represented by formula (K-1):

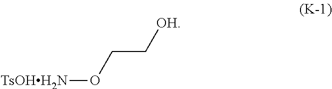

The compound of formula (K) or the salt thereof can be present in an excess amount relative to the compound of formula (II). In some embodiments, the compound of formula (K) or the salt thereof is present in an amount of from about 1.1 to about 5 equivalents, from about 1.1 to about 4 equivalents, from about 1.1 to about 3 equivalents, from about 1.1 to about 2 equivalents, or from about 1.1 to about 1.5 equivalents, relative to the compound of formula (II). In some embodiments, the compound of formula (K) or the salt thereof is present in an amount of from about 1.1 to about 1.5 equivalents relative to the compound of formula (II). In some embodiments, the compound of formula (K-1) is present in an amount of from about 1.1 to about 3 equivalents, from about 1.1 to about 2 equivalents, or from about 1.1 to about 1.5 equivalents, relative to the compound of formula (II). In some embodiments, the compound of formula (K-1) is present in an amount of from about 1.1 to about 1.5 equivalents relative to the compound of formula (II). In some embodiments, the compound of formula (K-1) is present in an amount of about 1.25 equivalents relative to the compound of formula (II).

The silylating agent can be any trialkyl silylating agent. In some embodiments, the silylating agent is a trialkyl silylating agent. In some embodiments, the silylating agent is triethyl silylating agent or trimethyl silylating agent. In some embodiments, the silylating agent is trimethylsilyl chloride (TMSCl).

The silylating agent can be present in an equal amount or in an excess amount relative to the compound of formula (K) or the salt thereof as described above. In some embodiments, the silylating agent is present in an amount of from about 1.2 to about 5.5 equivalents, from about 1.2 to about 4.4 equivalents, from about 1.2 to about 3.3 equivalents, from about 1.2 to about 2.2 equivalents, from about 1.2 to about 2.0 equivalents, or from about 1.2 to about 1.6 equivalents, relative to the compound of formula (II). In some embodiments, trimethylsilyl chloride (TMSCl) is present in an amount of from about 1.2 to about 3.3 equivalents, from about 1.2 to about 2.2 equivalents, from about 1.2 to about 2.0 equivalents, or from about 1.2 to about 1.6 equivalents relative to the compound of formula (II). In some embodiments, trimethylsilyl chloride (TMSCl) is present in an amount of from about 1.2 to about 2.0 equivalents relative to the compound of formula (II). In some embodiments, trimethylsilyl chloride (TMSCl) is present in an amount of from about 1.2 to about 1.6 equivalents relative to the compound of formula (II). In some embodiments, trimethylsilyl chloride (TMSCl) is present in an amount of about 1.35 equivalents relative to the compound of formula (II). In some embodiments, trimethylsilyl chloride (TMSCl) is present in an amount of about 1.7 equivalents relative to the compound of formula (II).

The first base can be an organic or inorganic base, as defined herein. In some embodiments, the first base is an organic base. In some embodiments, the first base is a tertiary amine. In some embodiments, the tertiary amine is triethylamine, tri-n-butylamine, N,N-diisopropylethylamine, N-methylpyrrolidine, N-methylmorpholine (also known as 4-methylmorpholine), dimethylaniline, diethylaniline, 1,8-bis(dimethylamino)naphthalene, quinuclidine, 1,4-diazabicylo[2.2.2]-octane (DABCO), or combinations thereof. In some embodiments, the tertiary amine is triethylamine, N,N-diisopropylethylamine, or 4-methylmorpholine. In some embodiments, the tertiary amine is triethylamine. In some embodiments, the tertiary amine is N,N-diisopropylethylamine. In some embodiments, the tertiary amine is 4-methylmorpholine. In some embodiments, the first base is triethylamine, N,N-diisopropylethylamine, or 4-methylmorpholine. In some embodiments, the first base is trimethylamine. In some embodiments, the first base is N,N-diisopropylethylamine. In some embodiments, the first base is 4-methylmorpholine.

The first base can be present in an excess amount relative to the compound of formula (II) and/or relative to the compound of formula (K) or the salt thereof. When the compound of formula (K) is in a salt form, an additional amount of the first base is required to neutralize the salt of the compound of formula (K).

When the compound of formula (K) is in a neutral form, in some embodiments, the first base is present in an amount of from about 2 to about 5 equivalents, from about 2 to about 4 equivalents, from about 3 to about 5 equivalents, or from about 3 to about 4 equivalents relative to the compound of formula (II). In some embodiments, the first base is present in an amount of from about 3 to about 5 equivalents relative to the compound of formula (II). In some embodiments, the first base is present in an amount of from about 3 to about 4 equivalents relative to the compound of formula (II). In some embodiments, triethylamine is present in an amount of from about 2 to about 5 equivalents, from about 2 to about 4 equivalents, from about 3 to about 5 equivalents, or from about 3 to about 4 equivalents relative to the compound of formula (II). In some embodiments, triethylamine is present in an amount of from about 3 to about 5 equivalents relative to the compound of formula (II). In some embodiments, triethylamine is present in an amount of from about 3 to about 4 equivalents relative to the compound of formula (II). In some embodiments, triethylamine is present in an amount of about 3.4 equivalents relative to the compound of formula (II). In some embodiments, N,N-diisopropylethylamine is present in an amount of from about 2 to about 5 equivalents, from about 2 to about 4 equivalents, from about 3 to about 5 equivalents, or from about 3 to about 4 equivalents relative to the compound of formula (II). In some embodiments, N,N-diisopropylethylamine is present in an amount of from about 3 to about 5 equivalents relative to the compound of formula (II). In some embodiments, N,N-diisopropylethylamine is present in an amount of from about 3 to about 4 equivalents relative to the compound of formula (II). In some embodiments, N,N-diisopropylethylamine is present in an amount of about 3.4 equivalents relative to the compound of formula (II). In some embodiments, 4-methylmorpholine is present in an amount of from about 2 to about 5 equivalents, from about 2 to about 4 equivalents, from about 3 to about 5 equivalents, or from about 3 to about 4 equivalents relative to the compound of formula (II). In some embodiments, 4-methylmorpholine is present in an amount of from about 3 to about 5 equivalents relative to the compound of formula (II). In some embodiments, 4-methylmorpholine is present in an amount of from about 3 to about 4 equivalents relative to the compound of formula (II). In some embodiments, 4-methylmorpholine is present in an amount of about 3.4 equivalents relative to the compound of formula (II).

When the compound of formula (K) is in a neutral form, in some embodiments, the first base is present in an amount of from about 2 to about 3 equivalents relative to the compound of formula (K). In some embodiments, triethylamine is present in an amount of from about 2 to about 3 equivalents relative to the compound of formula (K). In some embodiments, triethylamine is present in an amount of about 2.7 equivalents relative to the compound of formula (K). In some embodiments, N,N-diisopropylethylamine is present in an amount of from about 2 to about 3 equivalents relative to the compound of formula (K). In some embodiments, N,N-diisopropylethylamine is present in an amount of about 2.7 equivalents relative to the compound of formula (K). In some embodiments, 4-methylmorpholine is present in an amount of from about 2 to about 3 equivalents relative to the compound of formula (K). In some embodiments, 4-methylmorpholine is present in an amount of about 2.7 equivalents relative to the compound of formula (K).

When the compound of formula (K) is in a salt form, in some embodiments, the first base is present in an amount of from about 3 to about 6 equivalents, from about 3 to about 5 equivalents, from about 4 to about 6 equivalents, or from about 4 to about 5 equivalents relative to the compound of formula (II). In some embodiments, the first base is present in an amount of from about 4 to about 6 equivalents relative to the compound of formula (II). In some embodiments, the first base is present in an amount of from about 4 to about 5 equivalents relative to the compound of formula (II). In some embodiments, triethylamine is present in an amount of from about 3 to about 6 equivalents, from about 3 to about 5 equivalents, from about 4 to about 6 equivalents, or from about 4 to about 5 equivalents relative to the compound of formula (II). In some embodiments, triethylamine is present in an amount of from about 4 to about 6 equivalents relative to the compound of formula (II). In some embodiments, triethylamine is present in an amount of from about 4 to about 5 equivalents relative to the compound of formula (II). In some embodiments, triethylamine is present in an amount of about 4.4 equivalents relative to the compound of formula (II). In some embodiments, N,N-diisopropylethylamine is present in an amount of from about 3 to about 6 equivalents, from about 3 to about 5 equivalents, from about 4 to about 6 equivalents, or from about 4 to about 5 equivalents relative to the compound of formula (II). In some embodiments, N,N-diisopropylethylamine is present in an amount of from about 4 to about 6 equivalents relative to the compound of formula (II). In some embodiments, N,N-diisopropylethylamine is present in an amount of from about 4 to about 5 equivalents relative to the compound of formula (II). In some embodiments, N,N-diisopropylethylamine is present in an amount of about 4.4 equivalents relative to the compound of formula (II). In some embodiments, 4-methylmorpholine is present in an amount of from about 3 to about 6 equivalents, from about 3 to about 5 equivalents, from about 4 to about 6 equivalents, or from about 4 to about 5 equivalents relative to the compound of formula (II). In some embodiments, 4-methylmorpholine is present in an amount of from about 4 to about 6 equivalents relative to the compound of formula (II). In some embodiments, 4-methylmorpholine is present in an amount of from about 4 to about 5 equivalents relative to the compound of formula (II). In some embodiments, 4-methylmorpholine is present in an amount of about 4.4 equivalents relative to the compound of formula (II). In some embodiments, 4-methylmorpholine is present in an amount of about 5.5 equivalents relative to the compound of formula (II).

When the compound of formula (K) is in a salt form, in some embodiments, the first base is present in an amount of from about 3 to about 5 equivalents relative to the salt of the compound of formula (K). In some embodiments, triethylamine is present in an amount of from about 3 to about 5 equivalents relative to the salt of the compound of formula (K). In some embodiments, triethylamine is present in an amount of about 3.5 equivalents relative to the salt of the compound of formula (K). In some embodiments, N,N-diisopropylethylamine is present in an amount of from about 3 to about 5 equivalents relative to the salt of the compound of formula (K). In some embodiments, N,N-diisopropylethylamine is present in an amount of about 3.5 equivalents relative to the salt of the compound of formula (K). In some embodiments, 4-methylmorpholine is present in an amount of from about 3 to about 5 equivalents relative to the salt of the compound of formula (K). In some embodiments, 4-methylmorpholine is present in an amount of about 3.5 equivalents relative to the salt of the compound of formula (K). In some embodiments, 4-methylmorpholine is present in an amount of about 4.5 equivalents relative to the salt of the compound of formula (K). In some embodiments, the salt of the compound of formula (K) is a p-toluenesulfonic acid salt represented by formula (K-1).

In some embodiments, the compound of formula (K) or the salt thereof is present in an amount of from about 1.1 to about 1.5 equivalents relative to the compound of formula (II); trimethylsilyl chloride (TMSCl) is present in an amount of from about 1.2 to about 2.0 equivalents relative to the compound of formula (II); and 4-methylmorpholine is present in an amount of from about 3 to about 5 equivalents relative to the compound of formula (II), when the compound of formula (K) is in a neutral form; or 4-methylmorpholine is present in an amount of from about 4 to about 6 equivalents relative to the compound of formula (II), when the compound of formula (K) is in a salt form. When the compound of formula (K) is in a neutral form, in some embodiments, the compound of formula (K) is present in an amount of from about 1.1 to about 1.5 equivalents relative to the compound of formula (II); trimethylsilyl chloride (TMSCl) is present in an amount of from about 1.2 to about 2.0 equivalents relative to the compound of formula (II); and 4-methylmorpholine present in an amount of from about 3 to about 5 equivalents relative to the compound of formula (II). When the compound of formula (K) is in a neutral form, in some embodiments, the compound of formula (K) is present in an amount of about 1.25 equivalents relative to the compound of formula (II); trimethylsilyl chloride (TMSCl) is present in an amount of about 1.35 equivalents relative to the compound of formula (II); and 4-methylmorpholine is present in an amount of about 3.4 equivalents relative to the compound of formula (II). When the compound of formula (K) is in a salt form, in some embodiments, the salt of the compound of formula (K) is present in an amount of from about 1.1 to about 1.5 equivalents relative to the compound of formula (II); trimethylsilyl chloride (TMSCl) is present in an amount of from about 1.2 to about 2.0 equivalents relative to the compound of formula (II); and 4-methylmorpholine present in an amount of from about 4 to about 6 equivalents relative to the compound of formula (II). In some embodiments, the compound of formula (K-1) is present in an amount of from about 1.1 to about 1.5 equivalents relative to the compound of formula (II); trimethylsilyl chloride (TMSCl) is present in an amount of from about 1.2 to about 2.0 equivalents relative to the compound of formula (II); and 4-methylmorpholine present in an amount of from about 4 to about 6 equivalents relative to the compound of formula (II). In some embodiments, the compound of formula (K-1) is present in an amount of about 1.25 equivalents relative to the compound of formula (II); trimethylsilyl chloride (TMSCl) is present in an amount of about 1.35 equivalents relative to the compound of formula (II); and 4-methylmorpholine present in an amount of about 4.4 equivalents relative to the compound of formula (II). In some embodiments, the compound of formula (K-1) is present in an amount of about 1.25 equivalents relative to the compound of formula (II); trimethylsilyl chloride (TMSCl) is present in an amount of about 1.7 equivalents relative to the compound of formula (II); and 4-methylmorpholine present in an amount of about 5.5 equivalents relative to the compound of formula (II).

The first solvent in step 6a) can be an aprotic solvent as defined herein. In some embodiments, the first solvent is tetrahydrofuran (THF), 2-methyltetrahydrofuran (2-MeTHF), acetonitrile (ACN), dichloromethane (DCM), methyl tert-butyl ether (MTBE), heptanes, isopropyl acetate (IPAc), or combinations thereof. In some embodiments, the first solvent is tetrahydrofuran (THF), 2-methyltetrahydrofuran (2-MeTHF), methyl tert-butyl ether (MTBE), or combinations thereof. In some embodiments, the first solvent includes tetrahydrofuran (THF). In some embodiments, the first solvent is tetrahydrofuran (THF). In some embodiments, the first solvent includes methyl tert-butyl ether (MTBE). In some embodiments, the first solvent is methyl tert-butyl ether (MTBE).

In step 6a), prior to contacting the silylating agent, the compound of formula (K) or (K-1) can be first contacted with the first base in the first solvent, wherein the first solvent and base are each defined and described herein. In some embodiments, prior to contacting the silylating agent, the compound of formula (K) or (K-1) is first contacted with the first base in the first solvent. In some embodiments, prior to contacting the silylating agent, the compound of formula (K) or (K-1) is first contacted with 4-methylmorpholine in methyl tert-butyl ether (MTBE). In some embodiments, prior to contacting the silylating agent, the compound of formula (K-1) is first contacted with 4-methylmorpholine in methyl tert-butyl ether (MTBE). In some embodiments, prior to contacting the silylating agent, the compound of formula (K-1) is first contacted with 4-methylmorpholine in methyl tert-butyl ether (MTBE) to form a mixture including a precipitate that includes a p-toluenesulfonic acid salt of 4-methylmorpholine. In some embodiments, the precipitate including the p-toluenesulfonic acid salt of 4-methylmorpholine is filtered prior to contacting the silylating agent.

In step 6a), the compound of formula (K) in the neutral form can be a solution including the first solvent and the first base, wherein the solution can be prepared by contacting the salt of the compound of formula (K) (e.g., the compound of formula (K-1)) with the first base in the first solvent; and the first solvent and base are each defined and described herein. In some embodiments, the compound of formula (K) in the neutral form is a solution including 4-methylmorpholine and methyl tert-butyl ether (MTBE), which is prepared by contacting the compound of formula (K-1) with 4-methylmorpholine in methyl tert-butyl ether (MTBE) followed by filtering a precipitate including a p-toluenesulfonic acid salt of 4-methylmorpholine.

In some embodiments, the O-silyl protected compound of formula (K) in the first mixture is a compound represented by the formula:

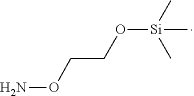

In some embodiments, the first mixture includes an O-silyl protected compound of formula (K) represented by the formula:

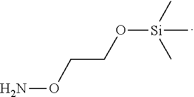

The first mixture of step 6a) can be formed separately or formed in-situ. In some embodiments, the first mixture of step 6a) is formed in-situ. In some embodiments, the first mixture of step 6a) is formed in-situ and is directly used for Step 6b).

The second mixture including the compound of formula (II) or a salt thereof can further includes a second solvent. In some embodiments, the second mixture further includes a second solvent.

The second solvent of can be an aprotic solvent as defined herein. In some embodiments, the second solvent is tetrahydrofuran (THF), 2-methyltetrahydrofuran (2-MeTHF), acetonitrile (ACN), dichloromethane (DCM), methyl tert-butyl ether (MTBE), heptanes, isopropyl acetate (IPAc), or combinations thereof. In some embodiments, the second solvent is tetrahydrofuran (THF), 2-methyltetrahydrofuran (2-MeTHF), methyl tert-butyl ether (MTBE), heptanes, isopropyl acetate (IPAc), or combinations thereof. In some embodiments, the second solvent includes tetrahydrofuran (THF). In some embodiments, the second solvent is tetrahydrofuran (THF). In some embodiments, the second solvent is methyl tert-butyl ether (MTBE), heptanes, or isopropyl acetate (IPAc). In some embodiments, the second solvent includes methyl tert-butyl ether (MTBE). In some embodiments, the second solvent is methyl tert-butyl ether (MTBE).

The compound of formula (II) can be in a salt form. In some embodiments, the compound of formula (II) is a HCl salt thereof.

In some embodiments, the second mixture includes the HCl salt of formula (II). In some embodiments, the second mixture includes the HCl salt of formula (II) and tetrahydrofuran (THF). In some embodiments, the second mixture includes the HCl salt of formula (II) and methyl tert-butyl ether (MTBE). In some embodiments, the second mixture is a slurry including the HCl salt of formula (II). In some embodiments, the second mixture is a slurry including the HCl salt of formula (II) and tetrahydrofuran (THF). In some embodiments, the second mixture is a slurry including the HCl salt of formula (II) and methyl tert-butyl ether (MTBE).

The second mixture can be added slowly over a period of time (e.g., 0.5 to 2 hours) so that the reaction mixture of Step 6b) is maintained at a temperature of no more than about 10° C. In some embodiments, the second mixture is added slowly over a period of about 0.5 to about 2 hours. In some embodiments, the second mixture is added slowly over a period of time while maintaining a temperature of no more than about 10° C. in step 6b). In some embodiments, the second mixture is added slowly over a period of about 0.5 to about 2 hours while maintaining a temperature of no more than about 10° C. in step 6b). In some embodiments, the second mixture including the HCl salt of formula (II) and tetrahydrofuran (THF) is added slowly over a period of time while maintaining a temperature of no more than about 10° C. in step 6b). In some embodiments, the second mixture including the HCl salt of formula (II) and tetrahydrofuran (THF) is added slowly over a period of about 0.5 to about 2 hours while maintaining a temperature of no more than about 10° C. in step 6b). In some embodiments, the second mixture including the HCl salt of formula (II) and methyl tert-butyl ether (MTBE) is added slowly over a period of time while maintaining a temperature of no more than about 10° C. in step 6b). In some embodiments, the second mixture including the HCl salt of formula (II) and methyl tert-butyl ether (MTBE) is added slowly over a period of about 0.5 to about 2 hours while maintaining a temperature of no more than about 10° C. in step 6b).

In general, steps 6a) and 6b) can be performed at any suitable temperature. In some embodiments, steps 6a) and 6b) are each conducted at a temperature of no more than about 10° C. In some embodiments, steps 6a) and 6b) are each conducted at a temperature of from about −5° C. to about 10° C. or from about −5° C. to about 5° C. In some embodiments, steps 6a) and 6b) are each conducted at a temperature of from about −5° C. to about 10° C. In some embodiments, steps 6a) and 6b) are each conducted at a temperature of from about 0° C. to about 10° C. In some embodiments, steps 6a) and 6b) are each conducted at a temperature of from about −5° C. to about 5° C.

The compound of formula (I) can be isolated by various methods (e.g., solvent exchange, precipitating, and/or recrystallization). In some embodiments, the compound of formula (I) is isolated by steps including: 6c) solvent exchanging; and 6d) precipitating. In some embodiments, step 6c) includes a solvent exchanging of a reaction mixture of step 6b) with ethanol. In some embodiments, step 6d) includes precipitating the compound of formula (I) from a mixture including ethanol and water. The treatment with active carbon can be performed prior to step 6c) and/or after step 6d). In some embodiments, the reaction mixture is first treated with active carbon prior to step 6c). In some embodiments, the precipitate including the compound of formula (I) from step 6d) is re-dissolved in a solvent and the resulted solution is then treated with active carbon.

### B. Step 5

In some embodiments, the process further includes prior to step 6a):
5) contacting a compound represented by formula (III):

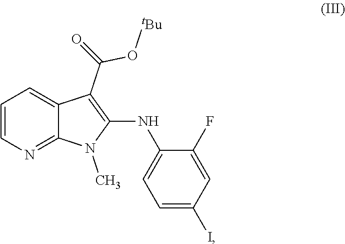 [SMILES: Cn1c(Nc2ccc(I)cc2F)c(C(=O)OC(C)(C)C)c2cccnc21]

or a salt thereof, with a first chlorinating agent and hydrogen chloride in a third solvent to form a HCl salt of the compound represented by formula (II):

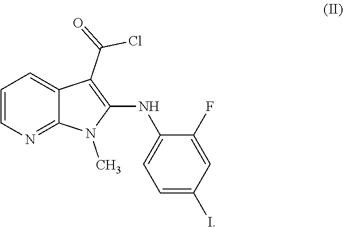

The first chlorinating agent can be a reagent capable of converting the —C(O)OBu group in the compound of formula (III) to corresponding —C(O)Cl. In some embodiments, the first chlorinating agent is phosphorous oxychloride, thionyl chloride, oxalyl chloride, sulfuryl chloride, or combinations thereof. In some embodiments, the first chlorinating agent is thionyl chloride or oxalyl chloride. In some embodiments, the first chlorinating agent is thionyl chloride.

The first chlorinating agent can be present in an excess amount relative to the compound of formula (III). In some embodiments, the first chlorinating agent is present in an excess amount of at least 5 equivalents relative to the compound of formula (III). In some embodiments, the first chlorinating agent is present in an amount of about 10 equivalents relative to the compound of formula (III). In some embodiments, the first chlorinating agent is thionyl chloride present in an amount of about 10 equivalents relative to the compound of formula (III).

The third solvent in step 5) can be an aprotic solvent as defined herein. In some embodiments, the third solvent is an ether. In some embodiments, the third solvent includes 1,4-dioxane.

Hydrogen chloride (HCl) can be a solution in the third solvent. In some embodiments, hydrogen chloride is a solution in 1,4-dioxane. In some embodiments, hydrogen chloride is a solution in 1,4-dioxane at a concentration of about 4 M.

Hydrogen chloride (HCl) can be present in an excess amount relative to the compound of formula (III). In some embodiments, hydrogen chloride is present in an amount of from about 5 to about 6 equivalents relative to the compound of formula (III). In some embodiments, hydrogen chloride is present in an amount of about 6 equivalents relative to the compound of formula (III).

In some embodiments, hydrogen chloride is a solution in 1,4-dioxane at a concentration of about 4 M; and hydrogen chloride is present in an amount of from about 5 to about 6 equivalents relative to the compound of formula (III). In some embodiments, hydrogen chloride is a solution in 1,4-dioxane at a concentration of about 4 M; and hydrogen chloride is present in an amount of about 6 equivalents relative to the compound of formula (III).

In general, step 5) can be performed at any suitable temperature. In some embodiments, step 5) is conducted at a temperature of from about 20° C. to about 60° C. In some embodiments, step 5) is conducted at a temperature of from about 30° C. to about 60° C., from 40° C. to about 60° C., or from 50° C. to about 60° C. In some embodiments, step 5) is conducted at a temperature of from 50° C. to about 60° C. In some embodiments, step 5) is conducted at a temperature of about 50° C.

The HCl salt of the compound of formula (II) can be isolated by various methods (e.g., solvent exchange and/or precipitating). In some embodiments, the HCl salt of formula (II) is isolated by steps including:
5a-1) diluting a reaction mixture of step 5) with a hydrocarbon solvent to form a slurry, or
5a-2) solvent-exchanging of a reaction mixture of step 5) with a hydrocarbon solvent to form a slurry;
5b) filtering the slurry to isolate a solid; and
5c) drying the solid under an inert gas to provide the HCl salt of formula (II).

In some embodiments, the hydrocarbon solvent includes n-heptane.

In some embodiments, the HCl salt of formula (II) is isolated by steps including:
5a-1) diluting a reaction mixture of step 5) with n-heptane to form a slurry;
5b) filtering the slurry to isolate a solid; and
5c) drying the solid under an inert gas to provide the HCl salt of formula (II).

In some embodiments, the HCl salt of formula (II) is isolated by steps including:
5a-2) solvent-exchanging of a reaction mixture of step 5) with n-heptane to form a slurry;
5b) filtering the slurry to isolate a solid; and
5c) drying the solid under an inert gas to provide the HCl salt of formula (II).

The inert gas can be nitrogen or argon gas; and the drying can be conducted under vacuum. In some embodiments, the inert gas is nitrogen gas and the drying is conducted under vacuum.

### C. Step 4

In some embodiments, the process further includes prior to step 5):
4a) contacting a compound represented by formula (V):

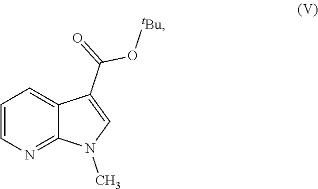

or a salt thereof, with a second chlorinating agent and a second base in a fourth solvent to form a compound represented by formula (IVa):

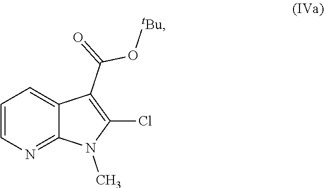

or a salt thereof, or
contacting a compound represented by formula (V) or a salt thereof with a iodinating agent and a second base in a fourth solvent to form a compound represented by formula (IVb):

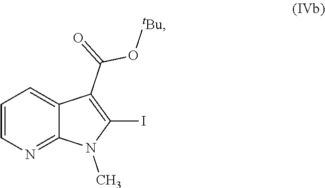

or a salt thereof; and
4b) reacting the compound of formula (IVa) or (IVb), or the salt thereof, with an aniline represented by formula (L):

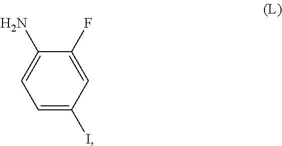

or a salt thereof, with a third base in a fifth solvent to form the compound represented by formula (III):

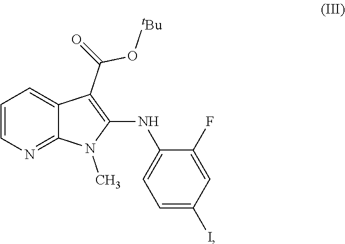 [SMILES: Cn1c(Nc2ccc(I)cc2F)c(C(=O)OC(C)(C)C)c2cccnc21]

or the salt thereof.

In some embodiments, the process further includes prior to step 5):
4a) contacting a compound represented by formula (V):

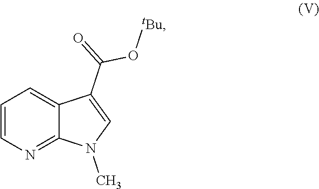 [SMILES: Cn1cc(C(=O)OC(C)(C)C)c2cccnc21]

or a salt thereof, with a second chlorinating agent and a second base in a fourth solvent to form a compound represented by formula (IVa):

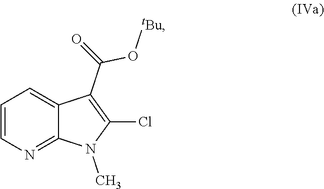 [SMILES: Cn1c(Cl)c(C(=O)OC(C)(C)C)c2cccnc21]

or a salt thereof; and
4b) reacting the compound of formula (IVa), or the salt thereof, with an aniline represented by formula (L):

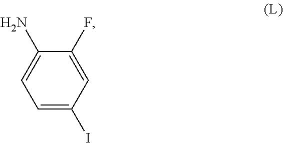

or a salt thereof, with a third base in a fifth solvent to form the compound represented by formula (III):

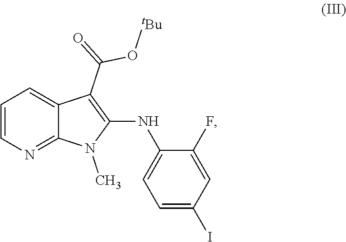

or the salt thereof.

With reference to step 4a) via the compound of formula (IVa), the second chlorinating agent can be a reagent capable of adding a chloro group, —Cl, at the 2-position of tert-butyl 1-methyl-1H-pyrrolo[2,3-b]pyridine-3-carboxylate of formula (V). In some embodiments, the second chlorinating agent is phosphorous oxychloride, thionyl chloride, oxalyl chloride, hexachloroethane, tosyl chloride, or combinations thereof. In some embodiments, the second chlorinating agent is hexachloroethane or tosyl chloride. In some embodiments, the second chlorinating agent is hexachloroethane.

The second chlorinating agent can be present in an amount of at least 1 equivalent relative to the compound of formula (V). In some embodiments, the second chlorinating agent is present in an amount of from about 1.1 to about 1.5 equivalents relative to the compound of formula (V). In some embodiments, the second chlorinating agent is present in an amount of about 1.1 equivalents relative to the compound of formula (V). In some embodiments, hexachloroethane is present in an amount of from about 1.1 to about 1.5 equivalents relative to the compound of formula (V). In some embodiments, hexachloroethane is present in an amount of about 1.1 equivalents relative to the compound of formula (V).

The second and third bases can be each independently a metal amide, an alkali alkoxide, or a combination thereof, wherein the metal amide and the alkali alkoxide are each defined and described herein.

The second base in step 4a) and the third base in step 4b) are each independently a metal amide as defined herein. In some embodiments, the second and third bases are each independently a metal amide. In some embodiments, the second base is a first metal amide and the third base is a second metal amide, wherein the first and second metal amides are the same. In some embodiments, the second base is a first metal amide and the third base is a second metal amide, wherein the first and second metal amides are different. In some embodiments, the metal amide is lithium diisopropylamide (LDA), lithium bis(trimethylsilyl)amide (LiHMDS), potassium bis(trimethylsilyl)amide (KHMDS), or lithium 2,2,6,6,-tetramethylpiperidide (LiTMP). In some embodiments, the second and third bases include each lithium bis(trimethylsilyl)amide (LiHMDS). In some embodiments, the second and third bases are each lithium bis(trimethylsilyl)amide (LiHMDS).

The second base in step 4a) is a metal amide as defined herein; and the third base in step 4b) includes an alkali alkoxide (e.g., an alkali tert-butoxide) as defined herein. In some embodiments, the second base in step 4a) is a metal amide; and the third base in step 4b) includes an alkali alkoxide. In some embodiments, the second base in step 4a) is a metal amide; and the third base in step 4b) includes an alkali tert-butoxide. In some embodiments, the metal amide is lithium diisopropylamide (LDA), lithium bis(trimethylsilyl)amide (LiHMDS), potassium bis(trimethylsilyl)amide (KHMDS), or lithium 2,2,6,6,-tetramethylpiperidide (LiTMP). In some embodiments, the alkali tert-butoxide is sodium tert-butoxide or potassium tert-butoxide. In some embodiments, the second base in step 4a) includes lithium bis(trimethylsilyl)amide (LiHMDS); and the third base in step 4b) includes potassium tert-butoxide. In some embodiments, the second base in step 4a) is lithium bis(trimethylsilyl)amide (LiHMDS); and the third base in step 4b) includes potassium tert-butoxide. In some embodiments, the second base in step 4a) is lithium bis(trimethylsilyl)amide (LiHMDS); and the third base in step 4b) is potassium tert-butoxide.

The second and third base can be added separately in each of steps 4a) and 4b), when steps 4a) and 4b) are conducted in one-pot or in two steps. Alternative, when the second and third bases are the same and steps 4a) and 4b) are conducted in one-pot, the total amount of combined second and third bases can be added once in step 4a).

When the second and third bases are added separately, the second base is present in an amount of at least 1 equivalent relative to the compound of formula (V). In some embodiments, the second base is present in an amount of from about 1.1 to about 2 equivalents relative to the compound of formula (V). In some embodiments, the second base is present in an amount of from about 1.1 to about 1.5 equivalents relative to the compound of formula (V). In some embodiments, the second base is lithium bis(trimethylsilyl)amide (LiHMIDS) in an amount of from about 1.1 to about 1.5 equivalents relative to the compound of formula (V). In some embodiments, the second base is lithium bis(trimethylsilyl)amide (LiHMIDS) in an amount of about 1.1 or about 1.2 equivalents relative to the compound of formula (V).

When the second and third bases are added separately, the third base is present in an amount of at least 2 equivalents relative to the compound of formula (V). In some embodiments, the third base is present in an amount of from about 2 to about 3.5 equivalents relative to the compound of formula (V). In some embodiments, the third base is present in an amount of from about 2 to about 2.5 equivalents relative to the compound of formula (V). In some embodiments, the third base is lithium bis(trimethylsilyl)amide (LiHMDS) in an amount of from about 2 to about 2.5 equivalents relative to the compound of formula (V). In some embodiments, the third base is lithium bis(trimethylsilyl)amide (LiHMIDS) in an amount of about 2.3 equivalents relative to the compound of formula (V). In some embodiments, the third base is potassium tert-butoxide in an amount of from about 2.5 to about 3.5 equivalents relative to the compound of formula (V). In some embodiments, the third base is potassium tert-butoxide in an amount of about 3 equivalents relative to the compound of formula (V).

In some embodiments, steps 4a) and 4b) are conducted in one-pot.

When steps 4a) and 4b) are conducted in one-pot, the third base is a part of the second base; and a total amount of combined second and third bases (as the second base) is added in step 4a). In some embodiments, the second and third bases are the same base in a total amount of from about 3 to about 4 equivalents relative to the compound of formula (V); and the total amount is added in step 4a). In some embodiments, the second and third bases are the same base in a total amount of about 3.5 equivalents relative to the compound of formula (V); and the total amount is added in step 4a). In some embodiments, the second and third bases are each lithium bis(trimethylsilyl)amide (LiHMDS) in a total amount of from about 3 to about 4 equivalents relative to the compound of formula (V); and the total amount is added in step 4a). In some embodiments, the second and third bases are each lithium bis(trimethylsilyl)amide (LiHMDS) in a total amount of about 3.5 equivalents relative to the compound of formula (V); and the total amount is added in step 4a).

In some embodiments, the process further includes prior to step 5):
4a) contacting a compound represented by formula (V):

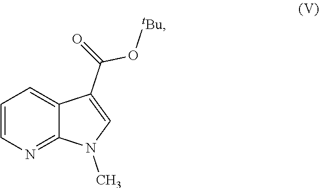 [SMILES: Cn1cc(C(=O)OC(C)(C)C)c2cccnc21]

or a salt thereof, with a iodinating agent and a second base in a fourth solvent to form a compound represented by formula (IVb):

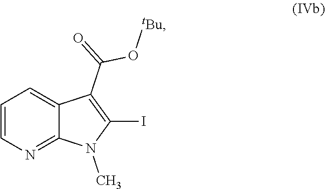 [SMILES: Cn1c(I)c(C(=O)OC(C)(C)C)c2cccnc21]

or a salt thereof; and
4b) reacting the compound of formula (IVb), or the salt thereof, with an aniline represented by formula (L):

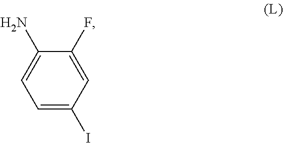

or a salt thereof, with a third base in a fifth solvent to form the compound represented by formula (III):

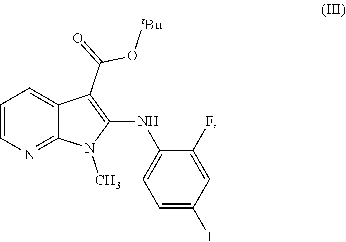

or the salt thereof.

With reference to step 4a) via the compound of formula (IVb), the iodinating agent can be a reagent capable of adding a iodo group, —I, at the 2-position of tert-butyl 1-methyl-1H-pyrrolo[2,3-b]pyridine-3-carboxylate of formula (V). In some embodiments, the iodinating agent is an in-situ iodinating agent. In some embodiments, when the second base is lithium bis(trimethylsilyl)amide (LiHMDS), the iodinating agent is an in-situ iodinating agent represented by the formula:

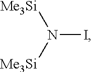

formed by reacting lithium bis(trimethylsilyl)amide (LiHMDS) with iodine.

In some embodiments, the compound of formula (IVb) is formed by adding a mixture including the compound of formula (V) or the salt thereof and iodine to lithium bis(trimethylsilyl)amide (LiHMDS) or a solution thereof.

In some embodiments, iodine is present in an amount of from about 1.05 to about 1.2 equivalents relative to the compound of formula (V).

With reference to steps 4a) and 4b) via the compound of formula (IVb), the second and third bases, and the additions thereof are described above. In some embodiments, lithium bis(trimethylsilyl)amide (LiHMDS) (as the second and third bases) can be added separately in each of steps 4a) and 4b) or added once in step 4a), as described herein.

When the second base is lithium bis(trimethylsilyl)amide (LiHMDS) which is added separately, in some embodiments, lithium bis(trimethylsilyl)amide (LiHMDS) is present in step 4a) in an amount of from about 1.1 to about 1.5 equivalents relative to the compound of formula (V). In some embodiments, lithium bis(trimethylsilyl)amide (LiHMDS) is present in step 4a) in an amount of about 1.1 equivalents relative to the compound of formula (V).

When the third base is lithium bis(trimethylsilyl)amide (LiHMDS) which is added separately, in some embodiments, lithium bis(trimethylsilyl)amide (LiHMDS) is present in step 4b) in an amount of from about 2 to about 2.5 equivalents relative to the compound of formula (V). In some embodiments, lithium bis(trimethylsilyl)amide (LiHMDS) is present in step 4b) in an amount of about 2.3 equivalents relative to the compound of formula (V).

When steps 4a) and 4b) are conducted in one-pot, in some embodiments, the second and third bases are each lithium bis(trimethylsilyl)amide (LiHMDS) in a total amount of from about 3 to about 4 equivalents relative to the compound of formula (V); and the total amount is added in step 4a). In some embodiments, the second and third bases are each lithium bis(trimethylsilyl)amide (LiHMDS) in a total amount of about 3.5 equivalents relative to the compound of formula (V); and the total amount is added in step 4a).

With reference to step 4b), in order to avoid an excess of the aniline of formula (L) at the end of step 4b), the aniline of formula (L) is preferred to be in an amount of about 0.9 to about 1.1 equivalents relative to the compound of formula (IVa) or (IVb). In some embodiments, the aniline of formula (L) is present in an amount of no more than 1.1 equivalent relative to the compound of formula (IVa) or (IVb). In some embodiments, the aniline of formula (L) is present in an amount of from about 0.95 to about 1.1 equivalents relative to the compound of formula (IVa) or (IVb). In some embodiments, the aniline of formula (L) is present in an amount of about 1.05 equivalents relative to the compound of formula (IVa) or (IVb). In some embodiments, the aniline of formula (L) is present in an amount of no more than 1.1 equivalent relative to the compound of formula (IVa). In some embodiments, the aniline of formula (L) is present in an amount of from about 0.95 to about 1.1 equivalents relative to the compound of formula (IVa). In some embodiments, the aniline of formula (L) is present in an amount of about 1.05 equivalents relative to the compound of formula (IVa). In some embodiments, the aniline of formula (L) is present in an amount of no more than 1.1 equivalent relative to the compound of formula (IVb). In some embodiments, the aniline of formula (L) is present in an amount of from about 0.95 to about 1.1 equivalents relative to the compound of formula (IVb). In some embodiments, the aniline of formula (L) is present in an amount of about 1.05 equivalents relative to the compound of formula (IVb).

When steps 4a) and 4b) are conducted in two steps, in some embodiments, the aniline of formula (L) is added to the compound of formula (IVa) or (IVb), or the salt thereof, in the fifth solvent. When steps 4a) and 4b) are conducted in two steps, in some embodiments, the aniline of formula (L) is added to the compound of formula (IVa), or the salt thereof, in the fifth solvent. When steps 4a) and 4b) are conducted in two steps, in some embodiments, the aniline of formula (L) is added to the compound of formula (IVb), or the salt thereof, in the fifth solvent.

When steps 4a) and 4b) are conducted in one pot, in some embodiments, the aniline of formula (L) is added to a reaction mixture of step 4a) including the compound of formula (IVa) or (IVb), or the salt thereof. When steps 4a) and 4b) are conducted in one pot, in some embodiments, the aniline of formula (L) is added to a reaction mixture of step 4a) including the compound of formula (IVa), or the salt thereof. When steps 4a) and 4b) are conducted in one pot, in some embodiments, the aniline of formula (L) is added to a reaction mixture of step 4a) including the compound of formula (IVb), or the salt thereof.

When steps 4a) and 4b) are conducted in one pot, in some embodiments, the second and third bases are the same base in a total amount of from about 3 to about 4 equivalents relative to the compound of formula (V); the total amount is added in step 4a); the aniline of formula (L) is in an amount of from about 0.95 to about 1.1 equivalents relative to the compound of formula (IVa) or (IVb); and the aniline of formula (L) is added to a reaction mixture of step 4a) including the compound of formula (IVa) or (IVb), or the salt thereof. In some embodiments, the second and third bases are the same base in a total amount of about 3.5 equivalents relative to the compound of formula (V); the total amount is added in step 4a); the aniline of formula (L) is in an amount of about 1.05 equivalents relative to the compound of formula (IVa) or (IVb); and the aniline of formula (L) is added to a reaction mixture of step 4a) including the compound of formula (IVa) or (IVb), or the salt thereof. In some embodiments, the second and third bases are each lithium bis(trimethylsilyl)amide (LiHMDS) in a total amount of from about 3 to about 4 equivalents relative to the compound of formula (V); the total amount is added in step 4a); the aniline of formula (L) is in an amount of from about 0.95 to about 1.1 equivalents relative to the compound of formula (IVa) or (IVb); and the aniline of formula (L) is added to a reaction mixture of step 4a) including the compound of formula (IVa) or (IVb), or the salt thereof. In some embodiments, the second and third bases are each lithium bis(trimethylsilyl)amide (LiHMDS) in a total amount of about 3.5 equivalents relative to the compound of formula (V); the total amount is added in step 4a); the aniline of formula (L) is in an amount of about 1.05 equivalents relative to the compound of formula (IVa) or (IVb); and the aniline of formula (L) is added to a reaction mixture of step 4a) including the compound of formula (IVa) or (IVb), or the salt thereof. In some embodiments, the second and third bases are each lithium bis(trimethylsilyl)amide (LiHMDS) in a total amount of from about 3 to about 4 equivalents relative to the compound of formula (V); the total amount is added in step 4a); the aniline of formula (L) is in an amount of from about 0.95 to about 1.1 equivalents relative to the compound of formula (IVa); and the aniline of formula (L) is added to a reaction mixture of step 4a) including the compound of formula (IVa), or the salt thereof. In some embodiments, the second and third bases are each lithium bis(trimethylsilyl)amide (LiHMDS) in a total amount of about 3.5 equivalents relative to the compound of formula (V); the total amount is added in step 4a); the aniline of formula (L) is in an amount of about 1.05 equivalents relative to the compound of formula (IVa); and the aniline of formula (L) is added to a reaction mixture of step 4a) including the compound of formula (IVa), or the salt thereof.

The fourth solvent in step 4a) can be an aprotic solvent as defined herein. In some embodiments, the fourth solvent is an ether. In some embodiments, the fourth solvent includes tetrahydrofuran (THF).

The fifth solvent in step 4b) can be an aprotic solvent as defined herein. In some embodiments, the fifth solvent is an ether. In some embodiments, the fifth solvent includes tetrahydrofuran (THF).

In some embodiments, the fourth and fifth solvents include each tetrahydrofuran (THF). In some embodiments, the fourth and fifth solvents are each tetrahydrofuran (THF).

In general, steps 4a) and 4b) can be performed at any suitable temperature. In some embodiments, steps 4a) and 4b) are each conducted at a temperature of from about −5° C. to about 25° C. In some embodiments, step 4a) is conducted at a temperature of from about 0° C. to about 10° C. In some embodiments, step 4b) is conducted at a temperature of from 0° C. to about 25° C. In some embodiments, step 4b) is conducted at an initial temperature of from about 0° C. to about 10° C. and then warmed up to a temperature of from about 15° C. to about 25° C.

At the end of step 4b), in some embodiments, the reaction mixture of step 4b) is quenched with an aqueous solution of ammonium chloride.

The compound of formula (III) or a salt thereof can be isolated by various methods (e.g., solvent exchange and/or precipitating). In some embodiments, the compound of formula (III) or a salt thereof is isolated by steps including: 4c) solvent exchanging; and/or 4d) precipitating. In some embodiments, step 4c) includes a first solvent exchanging of a quenched mixture to a biphasic mixture including THF and water; and a second solvent-exchanging of the biphasic mixture with ethanol. In some embodiments, step 4d) includes precipitating the compound of formula (III) or a salt thereof from a mixture including ethanol and water. In some embodiments, the compound of formula (III) or a salt thereof is isolated by precipitating from a mixture including isopropanol and water (without a distillation of the reaction solvent, e.g., THF, and/or solvent exchanging).

### D. Step 3

In some embodiments, the process further include prior to step 4a):
3) converting a compound represented by formula (VI):

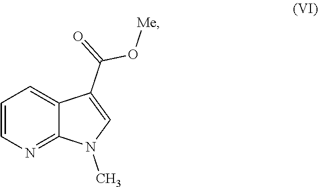

or a salt thereof to the compound represented by formula (V):

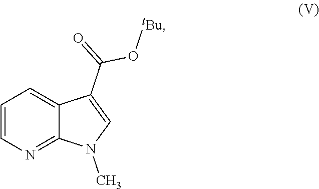 [SMILES: Cn1cc(C(=O)OC(C)(C)C)c2cccnc21]

or the salt thereof.

In some embodiments, step 3) is conducted with a salt of tert-butanol in a sixth solvent. In some embodiments, the salt of tert-butanol is sodium tert-butoxide.

The sixth solvent in step 3) can be an aprotic solvent as defined herein. In some embodiments, the sixth solvent is a non-polar solvent as defined herein. In some embodiments, the sixth solvent includes toluene. In some embodiments, the sixth solvent is toluene.

In general, step 3) can be conducted at any suitable temperature. In some embodiments, step 3) is conducted at a temperature of from about 95° C. to about 110° C. In some embodiments, step 3) is conducted at a temperature of from about 97° C. to about 107° C.

### E. Steps 1 and 2

In some embodiments, the process further include prior to step 3):
1a) N-methylating a compound represented by formula (IX):

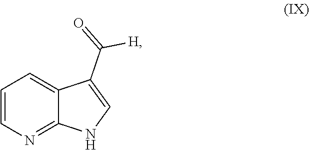

or a salt thereof to provide a compound represented by formula (VIII):

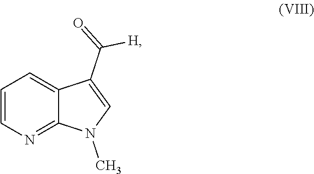

or a salt thereof;
1b) oxidizing the compound of formula (VIII) or the salt thereof to a compound represented by formula (VII):

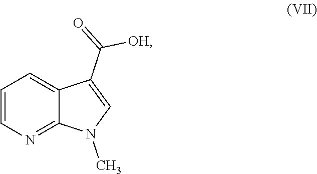

or a salt thereof; and
2) esterificating the compound of formula (VII) to provide the compound represented by formula (VI):

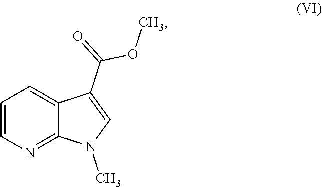

or the salt thereof.

In some embodiments, step 1a) is conducted with 1,4-diazabicyclo[2.2.2]octane (DABCO) and dimethyl carbonate in dimethylformamide (DMF). In some embodiments, DABCO is present in an amount of about 0.1 equivalent relative to the compound of formula (IX). In some embodiments, dimethyl carbonate and DMF has a ratio of 19 to 1 by volume.

In general, step 1a) can be conducted at any suitable temperature. In some embodiments, step 1a) is conducted at a temperature of from about 80° C. to about 86° C.

In some embodiments, step 1b) is conducted with sodium chlorite and sulfamic acid in water.

In general, step 1b) can be conducted at any suitable temperature. In some embodiments, step 1b) is conducted at a temperature of from about 0° C. to about 18° C.

In some embodiments, step 2) is conducted with methanol and sulfuric acid.

In general, step 2) can be conducted at any suitable temperature. In some embodiments, step 2) is conducted at a temperature of from about 58° C. to about 68° C.

In some embodiments, the compound of any one of formulae (I), (III), (IVa), (V), (VI), (VII), (VIII), and (IX) is in a salt form. In some embodiments, the compound of formula (II) in step 6b) is in a salt form. In some embodiments, the compound of formula (II) in step 5) is a HCl salt thereof.

Examples of applicable salt forms include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures), succinates, benzoates and salts with amino acids such as glutamic acid. These salts may be prepared by methods known to those skilled in art. When compounds of the present disclosure contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 23rd Edition, 2020, which is incorporated herein by reference.

Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19).

In some embodiments, the compound of any one of formulae (I), (III), (IVa), (V), (VI), (VII), (VIII), and (IX) is in a neutral form. In some embodiments, the compounds of formula (I) is in a neutral form.

### F. Selected Embodiments

In a second aspect, the present disclosure provides a process for preparing a compound represented by formula (I):

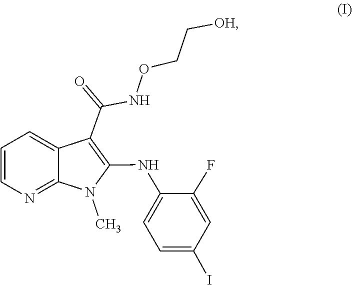 [SMILES: Cn1c(Nc2ccc(I)cc2F)c(C(=O)NOCCO)c2cccnc21]

or a salt thereof, the process including:
3) converting a compound represented by formula (VI):

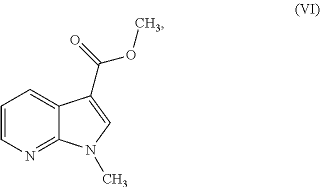

or a salt thereof, to a compound represented by formula (V):

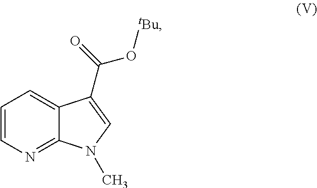

or a salt thereof, with sodium tert-butoxide in toluene;
4a) contacting the compound represented by formula (V) or the salt thereof with hexachloroethane and lithium bis(trimethylsilyl)amide (LiHMDS) in THF to form a compound represented by formula (IVa):

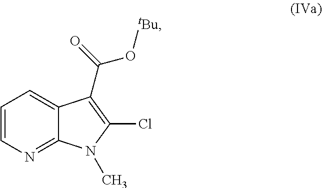

or a salt thereof;
4b) adding an aniline represented by formula (L):

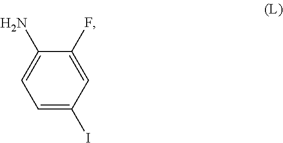

to a reaction mixture of step 4a) comprising the compound of formula (IVa) or the salt thereof to form a compound represented by formula (III):

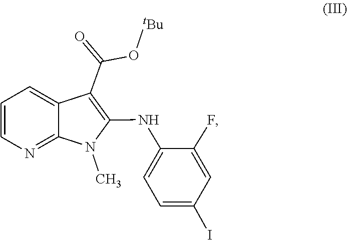 [SMILES: Cn1c(Nc2ccc(I)cc2F)c(C(=O)OC(C)(C)C)c2cccnc21]

or a salt thereof;
5) contacting the compound represented by formula (III) or the salt thereof with thionyl chloride and hydrogen chloride in a 1,4-dioxane to form a HCl salt of a compound represented by formula (II):

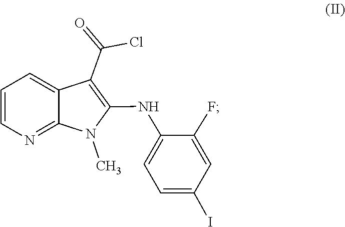 [SMILES: Cn1c(Nc2ccc(I)cc2F)c(C(=O)Cl)c2cccnc21]

6a) contacting a compound represented by formula (K) or a p-toluenesulfonic acid salt thereof represented by formula (K-1):

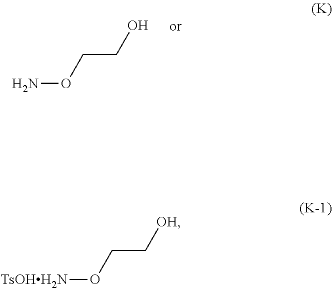

with 4-methylmorpholine and trimethylsilyl chloride (TMSCl) in tetrahydrofuran (THF) or methyl tert-butyl ether (MTBE) to form a first mixture; and
6b) adding a second mixture comprising the HCl salt of formula (II), and tetrahydrofuran (THF) or methyl tert-butyl ether (MTBE), to the first mixture of step 6a) to form the compound represented by formula (I) or the salt thereof.

With reference to step 3), in some embodiments, sodium tert-butoxide is present in an amount of about 2 equivalents relative to the compound of formula (VI). In some embodiments, step 3) is conducted at a temperature of from about 97° C. to about 107° C.

With reference to steps 4a) and 4b), in some embodiments, steps 4a) and 4b) are conducted in one-pot. In some embodiments, in step 4a), lithium bis(trimethylsilyl)amide (LiHMDS) is present in a total amount of about 3.5 equivalents relative to the compound of formula (V); and the total amount is added in step 4a). In some embodiments, in step 4a), hexachloroethane is present in an amount of about 1.1 equivalents relative to the compound of formula (V). In some embodiments, in step 4b), the aniline of formula (L) is present in an amount of about 0.98 equivalent relative to the compound of formula (IVa). In some embodiments, in step 4b), the aniline of formula (L) is present in an amount of about 1.05 equivalent relative to the compound of formula (IVa). In some embodiments, steps 4a) and 4b) are each conducted at a temperature of from about −5° C. to about 25° C.

The compound of formula (III) can be isolated as described herein. In some embodiments, a reaction mixture of step 4b) is quenched with an aqueous solution of ammonium chloride. In some embodiments, the compound of formula (III) or the salt thereof is isolated by steps including:
4c) a first solvent exchanging of a quenched mixture to a biphasic mixture including THF and water; and a second solvent-exchanging of the biphasic mixture with ethanol; and
4d) precipitating from a mixture including ethanol and water to provide the compound of formula (III) or the salt thereof.

In some embodiments, the compound of formula (III) or a salt thereof is isolated by precipitating from a mixture including isopropanol and water (without a distillation of the reaction solvent, e.g., THF, and/or solvent exchanging).

With reference to step 5), thionyl chloride is present in an amount of about 10 equivalents relative to the compound of formula (III). In some embodiments, hydrogen chloride is a solution in 1,4-dioxane. In some embodiments, hydrogen chloride is a solution in 1,4-dioxane at a concentration of about 4 M; and hydrogen chloride is present in an amount of about 6 equivalents relative to the compound of formula (III). In some embodiments, step 5) is conducted at a temperature of from about 50° C. to about 55° C.

The compound of formula (II) can be isolated as described herein. In some embodiments, the HCl salt of the compound of formula (II) is isolated by steps comprising:
5a-1) diluting a reaction mixture of step 5) with n-heptane to form a slurry, or
5a-2) solvent-exchanging a reaction mixture of step 5) with n-heptane to form a slurry;
5b) filtering the slurry to isolate a solid; and
5c) drying the solid under nitrogen gas and vacuum to provide the HCl salt of the compound of formula (II).

In some embodiments, the HCl salt of the compound of formula (II) is isolated by steps comprising:
5a-1) diluting a reaction mixture of step 5) with n-heptane to form a slurry;
5b) filtering the slurry to isolate a solid; and
5c) drying the solid under nitrogen gas and vacuum to provide the HCl salt of the compound of formula (II).

With reference to step 6a), in some embodiments, trimethylsilyl chloride (TMSCl) is present in an amount of about 1.35 equivalents relative to the compound of formula (II). In some embodiments, the compound of formula (K) is in a neutral form. In some embodiments, the compound of formula (K) is present in an amount of about 1.25 equivalents relative to the compound of formula (II). In some embodiments, when the compound of formula (K) is in a neutral form, 4-methylmorpholine is present in an amount of about 3.4 equivalents relative to the compound of formula (II). In some embodiments, the compound of formula (K) is the p-toluenesulfonic acid salt of formula (K-1). In some embodiments, the p-toluenesulfonic acid salt of formula (K-1) is present in an amount of about 1.25 equivalents relative to the compound of formula (II). In some embodiments, when the compound of formula (K) is the p-toluenesulfonic acid salt of formula (K-1), 4-methylmorpholine is present in an amount of about 4.4 equivalents relative to the compound of formula (II).

With reference to step 6a), in some embodiments, when the compound of formula (K) is the p-toluenesulfonic acid salt of formula (K-1), the compound of formula (K-1) is present in an amount of about 1.25 equivalents; 4-methylmorpholine is present in an amount of about 5.5 equivalents; and trimethylsilyl chloride (TMSCl) is present in an amount of about 1.7 equivalents, all of which are relative to the compound of formula (II). In some embodiments, prior to contacting the silylating agent, the compound of formula (K-1) is first contacted with 4-methylmorpholine in methyl tert-butyl ether (MTBE) to form a mixture including a precipitate that includes a p-toluenesulfonic acid salt of 4-methylmorpholine. In some embodiments, the precipitate including the p-toluenesulfonic acid salt of 4-methylmorpholine is filtered prior to contacting the silylating agent.

With reference to step 6a), in some embodiments, the compound of formula (K) in the neutral form is a solution including 4-methylmorpholine and methyl tert-butyl ether (MTBE), which is prepared by contacting the compound of formula (K-1) with 4-methylmorpholine in methyl tert-butyl ether (MTBE) followed by filtering a precipitate including a p-toluenesulfonic acid salt of 4-methylmorpholine.

In some embodiments, the first mixture is formed in-situ.

With reference to step 6b), in some embodiments, the second mixture includes methyl tert-butyl ether (MTBE). In some embodiments, the second mixture is a slurry including the HCl salt of formula (II) and methyl tert-butyl ether (MTBE). In some embodiments, the second mixture is added slowly over a period of about 0.5 to 2 hours while maintaining a temperature of no more than about 10° C. in step 6b).

In some embodiments, step 6a) is conducted in tetrahydrofuran (THF); and step 6b) is conducted in a mixture of tetrahydrofuran (THF) and methyl tert-butyl ether (MTBE). In some embodiments, steps 6a) and 6b) are each conducted in methyl tert-butyl ether (MTBE).

In some embodiments, steps 6a) and 6b) are each conducted at a temperature of from −5° C. to about 10° C.

The compound of formula (I) can be isolated as described herein. In some embodiments, the compound of formula (I) is isolated by steps including:
6c) solvent exchanging of a reaction mixture of step 6b) with ethanol;
6d) precipitating from a mixture comprising ethanol and water and filtering a precipitate to provide the compound of formula (I).

In some embodiments, the process further includes prior to step 3):
1a) contacting a compound represented by formula (IX):

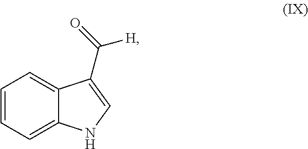

or a salt thereof, with dimethyl carbonate and 1,4-diazabicyclo[2.2. 2]octane (DABCO) in dimethylformamide to form a compound represented by formula (VIII):

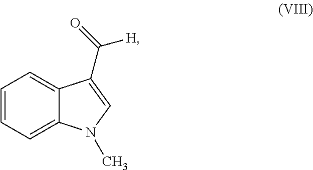

or a salt thereof;
1b) treating the compound of formula (VIII) or the salt thereof with sodium chlorite and sulfamic acid in water to form a compound represented by formula (VII):

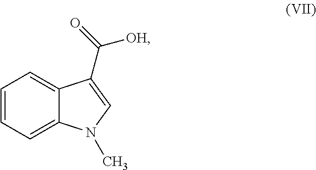

or a salt thereof; and
2) reacting the compound of formula (VII) or the salt thereof with methanol and sulfuric acid to provide the compound represented by formula (VI):

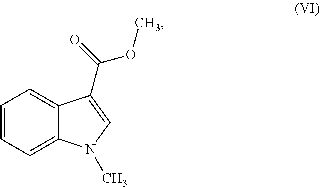

or the salt thereof.

With reference to step 1a), in some embodiments, DABCO is present in an amount of about 0.1 equivalent relative to the compound of formula (IX). In some embodiments, dimethyl carbonate and DMF has a ratio of 19 to 1 by volume. In some embodiments, step 1a) is conducted at a temperature of from about 80° C. to about 86° C.

In some embodiments, step 1b) is conducted at a temperature of from about 0° C. to about 18° C.

In some embodiments, step 2) is conducted at a temperature of from about 58° C. to about 68° C.

In some embodiments, the compound of any one of formulae (I), (III), (IVa), (V), (VI), (VII), (VIII), and (IX) is in a neutral form. In some embodiments, the compound of formula (I) is in neutral form.

### IV. Processes for Preparing a Compound of Formula (K)

In a third aspect, the present disclosure provides a process for preparing a compound represented by formula (K):

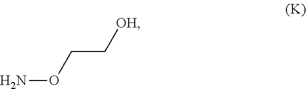

or a salt thereof, the process including:
7) contacting 2-hydroxyisoindoline-1,3-dione represented by the formula:

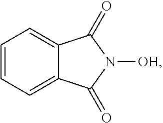 [SMILES: O=C1c2ccccc2C(=O)N1O]

with 2-bromoethanol and a non-nucleophilic base in an aprotic solvent to form
2-(2-hydroxyethoxy)isoindoline-1,3-dione represented by formula (J):

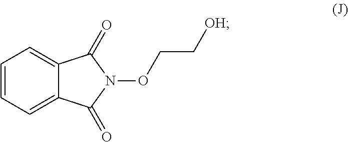 [SMILES: O=C1c2ccccc2C(=O)N1OCCO]

8a) treating 2-(2-hydroxyethoxy)isoindoline-1,3-dione with ammonia in an alcohol solvent to provide the compound of formula (K); and
8b) optionally converting the compound of formula (K) to the salt thereof.

In some embodiments, 2-bromoethanol in step 7) is present in an amount of from about 1.05 to about 1.5 equivalents relative to 2-hydroxyisoindoline-1,3-dione (also known as N-hydroxyphthalamide). In some embodiments, 2-bromoethanol is present in an amount of about 1.4 equivalents relative to 2-hydroxyisoindoline-1,3-dione. In some embodiments, 2-bromoethanol is present in an amount of about 1.2 equivalents relative to 2-hydroxyisoindoline-1,3-dione. In some embodiments, 2-bromoethanol is present in an amount of about 1.1 equivalents relative to 2-hydroxyisoindoline-1,3-dione.

In some embodiments, the non-nucleophilic base in step 7) is a tertiary amine as defined and described herein. In some embodiments, the tertiary amine in step 7) is triethylamine (TEA), tri-n-butylamine, N,N-diisopropylethylamine (DIPEA), N-methylpyrrolidine, N-methylmorpholine (also known as 4-methylmorpholine), dimethylaniline, diethylaniline, 1,8-bis(dimethylamino)naphthalene, quinuclidine, 1,4-diazabicylo[2.2.2]-octane (DABCO), or combinations thereof. In some embodiments, the tertiary amine is triethylamine or N,N-diisopropylethylamine. In some embodiments, the tertiary amine is triethylamine. In some embodiments, the tertiary amine is N,N-diisopropylethylamine.

In some embodiments, the tertiary amine in step 7) is present in an amount of from about 1.05 to about 1.5 equivalents or from about 1.05 to about 1.2 equivalents relative to 2-hydroxyisoindoline-1,3-dione. In some embodiments, triethyl amine is present in an amount of from about 1.05 to about 1.2 equivalents relative to 2-hydroxyisoindoline-1,3-dione. In some embodiments, triethyl amine is present in an amount of about 1.1 equivalents relative to 2-hydroxyisoindoline-1,3-dione. In some embodiments, triethyl amine is present in an amount of about 1.2 equivalents relative to 2-hydroxyisoindoline-1,3-dione. In some embodiments, N,N-diisopropylethylamine is present in an amount of from about 1.05 to about 1.2 equivalents relative to 2-hydroxyisoindoline-1,3-dione. In some embodiments, N,N-diisopropylethylamine is present in an amount of about 1.1 equivalents relative to 2-hydroxyisoindoline-1,3-dione. In some embodiments, N,N-diisopropylethylamine is present in an amount of about 1.2 equivalents relative to 2-hydroxyisoindoline-1,3-dione.

In some embodiments, 2-bromoethanol is present in an amount of about 1.4 equivalents and triethyl amine is present in an amount of about 1.2 equivalents, relative to 2-hydroxyisoindoline-1,3-dione. In some embodiments, 2-bromoethanol is present in an amount of about 1.2 equivalents and N,N-diisopropylethylamine is present in an amount of about 1.2 equivalents, relative to 2-hydroxyisoindoline-1,3-dione.

In some embodiments, the non-nucleophilic base in step 7) is an amidine-based compound (e.g., DBU or DBN). In some embodiments, the amidine-based compound in step 7) is 1,8-diazabicyclo[5.4.0]undec-7-ene (DBU) or 1,5-diazabicyclo[4.3.0]non-5-en (DBN). In some embodiments, the amidine-based compound is DBU.

In some embodiments, the amidine-based compound in step 7) is present in an amount of from about 1.0 to about 1.5 equivalents or from about 1.0 to about 1.2 equivalents relative to 2-hydroxyisoindoline-1,3-dione. In some embodiments, DBU is present in an amount of from about 1.0 to about 1.2 equivalents relative to 2-hydroxyisoindoline-1,3-dione. In some embodiments, DBU is present in an amount of about 1.0 equivalents relative to 2-hydroxyisoindoline-1,3-dione. In some embodiments, DBU is present in an amount of about 1.1 equivalents relative to 2-hydroxyisoindoline-1,3-dione. In some embodiments, DBU is present in an amount of about 1.2 equivalents relative to 2-hydroxyisoindoline-1,3-dione.

In some embodiments, 2-bromoethanol is present in an amount of about 1.1 equivalents and DBU is present in an amount of about 1.0 equivalents, relative to 2-hydroxyisoindoline-1,3-dione. In some embodiments, 2-bromoethanol is present in an amount of about 1.1 equivalents and DBU is present in an amount of about 1.1 equivalents, relative to 2-hydroxyisoindoline-1,3-dione. In some embodiments, 2-bromoethanol is present in an amount of about 1.2 equivalents and DBU is present in an amount of about 1.2 equivalents, relative to 2-hydroxyisoindoline-1,3-dione.

In some embodiments, when the non-nucleophilic base in step 7) is a tertiary amine (e.g., TEA or DIPEA), the aprotic solvent in step 7) is tetrahydrofuran (THF), 2-methyltetrahydrofuran (2-MeTHF), acetonitrile (ACN), dichloromethane (DCM), methyl tert-butyl ether (MTBE), heptanes, isopropyl acetate (IPAc), or combinations thereof. In some embodiments, the aprotic solvent includes acetonitrile (ACN). In some embodiments, the aprotic solvent is acetonitrile (ACN).

In some embodiments, when the non-nucleophilic base in step 7) is an amidine-based compound (e.g., DBU), the aprotic solvent in step 7) includes dimethylformamide (DMF). In some embodiments, when the non-nucleophilic base in step 7) is DBU, the aprotic solvent includes dimethylformamide (DMF). In some embodiments, when the non-nucleophilic base in step 7) is DBU, the aprotic solvent is dimethylformamide (DMF).

In general, step 7) can be conducted at any suitable temperature. In some embodiments, when the non-nucleophilic base in step 7) is a tertiary amine (e.g., TEA or DIPEA), step 7) is conducted at a temperature of from about 50° C. to about 100° C. In some embodiments, when the non-nucleophilic base in step 7) is a tertiary amine (e.g., TEA or DIPEA), step 7) is conducted at a temperature of from about 70° C. to about 80° C. In some embodiments, when the non-nucleophilic base in step 7) is an amidine-based compound (e.g., DBU), step 7) is conducted at a temperature of from about 20° C. to about 50° C. In some embodiments, when the non-nucleophilic base in step 7) is an amidine-based compound (e.g., DBU), step 7) is conducted at room temperature. In some embodiments, when the non-nucleophilic base in step 7) is an amidine-based compound (e.g., DBU), step 7) is conducted at a temperature of 40° C.

2-(2-hydroxyethoxy)isoindoline-1,3-dione of formula (J) from step 7) can be isolated by various methods (e.g., filtration, extraction, and/or precipitation).

In some embodiments, 2-(2-hydroxyethoxy)isoindoline-1,3-dione is isolated by steps including:
7a) filtering a solid comprising triethyl amine HBr salt to obtain a filtrate;
7b) adding water to the filtrate over a period of at least 1 hour to form a slurry; and
7c) filtering the slurry to isolate 2-(2-hydroxyethoxy)isoindoline-1,3-dione.

In some embodiments, 2-(2-hydroxyethoxy)isoindoline-1,3-dione is isolated by steps including:
7a) filtering a solid comprising triethyl amine HBr salt or N,N-diisopropylethylamine HBr salt to obtain a filtrate;
7b) extracting the filtrate with ethyl acetate (e.g., 3 times) followed by triturating with n-heptane to form a precipitation; and
7c) filtering the precipitation to isolate 2-(2-hydroxyethoxy)isoindoline-1,3-dione.

In some embodiments, when the base in step 7) is DBU, 2-(2-hydroxyethoxy)isoindoline-1,3-dione is isolated by steps including:
7a) extracting the reaction mixture of step 7) with ethyl acetate to provide an extract;
7b) concentrating the extract followed by precipitating from ethyl acetate and n-heptane; and
7c) filtering the precipitation to isolate 2-(2-hydroxyethoxy)isoindoline-1,3-dione.

In some embodiments, the alcohol solvent in step 8a) is methanol, ethanol, isopropanol, or combinations thereof. In some embodiments, the alcohol solvent includes methanol. In some embodiments, the alcohol solvent is methanol.

Ammonia in step 8a) can be a solution in the alcohol solvent as described herein. In some embodiments, ammonia is a solution in methanol. In some embodiments, ammonia is a solution in methanol at a concentration of from about 3.5 M to about 7 M. In some embodiments, ammonia is a solution in methanol at a concentration of about 3.5 M. In some embodiments, ammonia is a solution in methanol at a concentration of about 7 M.

In general, step 8a) can be conducted at any suitable temperature. In some embodiments, step 8a) is conducted at a temperature of from about 20° C. to 30° C.

The compound of formula (K) in a neutral form from step 8a) can be isolated by various methods. In some embodiments, the compound of formula (K) is isolated as a solution in isopropanol by steps including:
8a-1) filtering a reaction mixture of step 8a) to remove phthalimide byproduct thereby providing a filtrate; and
8a-2) solvent exchanging of the filtrate with isopropanol,
   wherein steps 8a-1) and 8a-2) are repeated at least once.

In some embodiments, in step 8b), the salt of the compound of formula (K) is a HCl, a sulfate, a hemisulfate, or a p-toluenesulfonic acid salt. In some embodiments, the salt of formula (K) is a p-toluenesulfonic acid salt represented by formula (K-1):

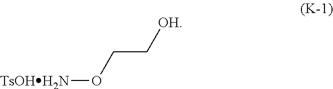

In some embodiments, the process of step 8b) includes:
8b-1) contacting the compound of formula (K) with p-toluenesulfonic acid in isopropanol;
8b-2) adding isopropyl acetate to a reaction mixture of step 8b-1) to precipitate the p-toluenesulfonic acid salt of formula (K-1).

In some embodiments, the compound of formula (K) is a solution in isopropanol prepared according to steps 8a-1) and 8a-2) as described above.

In some embodiments, p-toluenesulfonic acid in step 8b-1) is present in an amount of about 1.0 equivalent relative to the compound of formula (K).

In some embodiments, the p-toluenesulfonic acid salt of formula (K-1) is isolated as a solid by filtration followed by drying.

In general, step 8b-1) can be conducted at any suitable temperature. In some embodiments, step 8b-1) is conducted at a temperature of from about 35° C. to 45° C. In some embodiments, step 8b-1) is conducted at a temperature of about 40° C.

In some embodiments, the present disclosure provides a process for preparing a compound represented by formula (K-1):

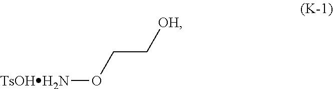

the process including:
7) contacting 2-hydroxyisoindoline-1,3-dione represented by the formula:

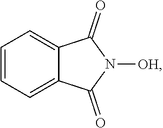 [SMILES: O=C1c2ccccc2C(=O)N1O]

with 2-bromoethanol and triethylamine in acetonitrile to form 2-(2-hydroxyethoxy)isoindoline-1,3-dione represented by the formula:

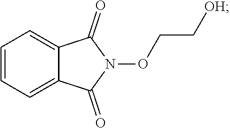 [SMILES: O=C1c2ccccc2C(=O)N1OCCO]

8a) treating 2-(2-hydroxyethoxy)isoindoline-1,3-dione with ammonia in methanol to provide a compound of formula (K):

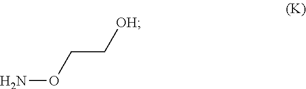

8a-1) filtering a reaction mixture of step 8a) to remove phthalimide byproduct thereby providing a filtrate;
8a-2) solvent exchanging of the filtrate with isopropanol, wherein steps 8a-1) and 8a-2) are repeated at least once;
8b-1) contacting the compound of formula (K) with p-toluenesulfonic acid in isopropanol;
8b-2) adding isopropyl acetate to a reaction mixture of step 8b-1) to precipitate the p-toluenesulfonic acid salt of formula (K-1).

In some embodiments, the compound of formula (K) in step 8b-1) is a solution in isopropanol from step 8a-2).

The reaction conditions for steps 7), 8a), and (8b-1) are as described herein. In some embodiments, 2-bromoethanol in step 7) is present in an amount of from about 1.4 equivalents relative to 2-hydroxyisoindoline-1,3-dione; triethyl amine in step 7) is present in an amount of about 1.1 equivalents relative to 2-hydroxyisoindoline-1,3-dione; ammonia in step 8a) is a solution in methanol at a concentration of from about 3.5 M; the compound of formula (K) in step 8b-1) is a solution in isopropanol from step 8a-2); and p-toluenesulfonic acid in step (8b-1) is present in an amount of about 1.0 equivalent relative to the compound of formula (K).

### V. PROCESSES FOR PREPARING MEK Inhibitors

In a fourth aspect, the present disclosure provides a process for preparing a MEK inhibitor represented by formula (XI):

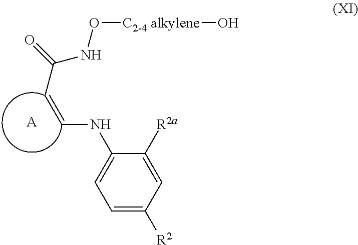

or a salt thereof, the process including:
a) contacting a compound of H₂N—O—C₂₋₄ alkylene-OH or a salt thereof, with a first base and a silylating agent in a first solvent to form a first mixture including an O-silyl protected compound thereof; and
b) reacting the first mixture with a compound represented by formula (XII):

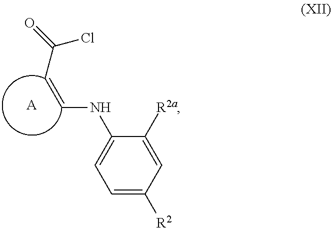

or a salt therefore to form the compound represented by formula (XI),
   wherein:
A ring is C₆₋₁₂ aryl or a 5-10 membered heteroaryl having 1 to 4 heteroatoms or groups as ring vertices independently selected from N, C(O), O, and S, each of which is unsubstituted or substituted; and
R² and R^{2a} are each independently halo, C₁₋₆ alkyl, —S—C₁₋₆ alkyl, C₂₋₆ alkenyl, or C₂₋₆ alkynyl.

In a fifth aspect, the present disclosure provides a process for preparing a MEK inhibitor represented by formula (XI):

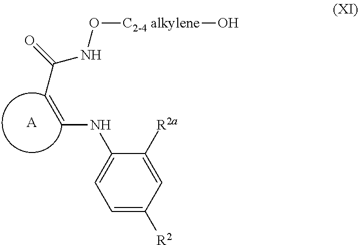

or a salt thereof, the process including:
a) contacting a compound of H₂N—O—C₂₋₄ alkylene-OH or a salt thereof, with a first base and a silylating agent in a first solvent to form a first mixture including an O-silyl protected compound thereof; and
b) reacting the first mixture with a compound represented by formula (XIII):

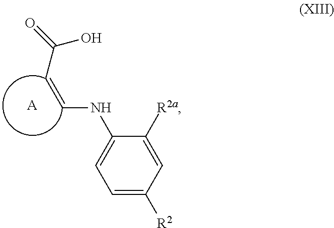

or a salt therefore to form the compound represented by formula (XI),
   wherein:
A ring is C₆₋₁₂ aryl or a 5-10 membered heteroaryl having 1 to 4 heteroatoms or groups as ring vertices independently selected from N, C(O), O, and S, each of which is unsubstituted or substituted; and
R² and R^{2a} are each independently halo, C₁₋₆ alkyl, —S—C₁₋₆ alkyl, C₂₋₆ alkenyl, or C₂₋₆ alkynyl.

With reference to any one of formulae (XI), (XII) and (XIII), in some embodiments, A ring is a 9-10 membered bicyclic heteroaryl having 1 to 3 heteroatoms or groups as ring vertices independently selected from N, C(O), O, and S, which is unsubstituted or substituted with one or more R groups; and each R group is independently CN, halo, C₁₋₆ alkyl, or C₁₋₆ alkoxy.

With reference to any one of formulae (XI), (XII) and (XIII), in some embodiments, A ring is a 5-6 membered monocyclic heteroaryl having 1 to 2 heteroatoms or groups as ring vertices independently selected from N, C(O), O, and S, which is unsubstituted or substituted with one or more R groups; and each R group is independently CN, halo, C₁₋₆ alkyl, C₁₋₆ alkoxy, or C₁₋₆ alkyl-C(O); or two adjacent R groups together form CH₂CH₂C(O) or CH₂CH₂CH₂C(O).

With reference to any one of formulae (XI), (XII) and (XIII), in some embodiments, A ring is phenyl, which is unsubstituted or substituted with one or more R groups; and each R group is independently CN, halo, C₁₋₆ alkyl, or C₁₋₆ alkoxy.

With reference to any one of formulae (XI), (XII) and (XIII), in some embodiments, A ring is selected from the group consisting of:

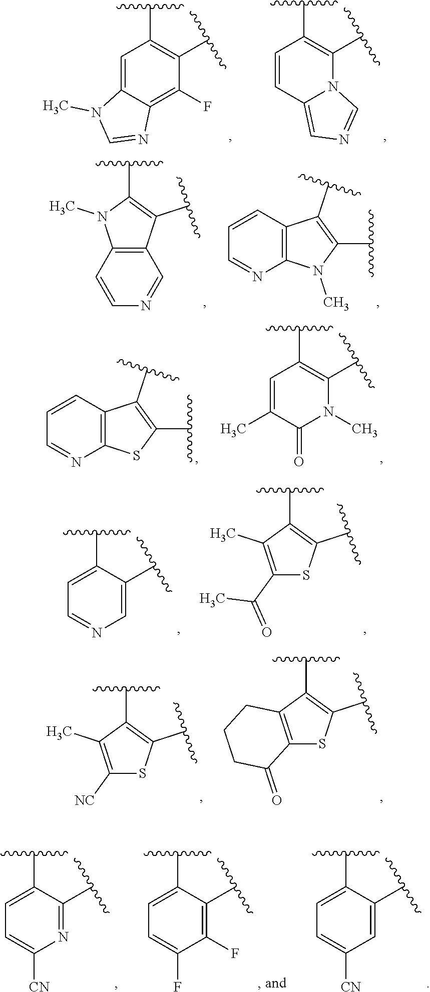

each of which is substituted with 0-3 R groups; and each R group is independently CN, F, Me, or OMe.

In some embodiments, in step a), the salt of H₂N—O—C₂₋₄ alkylene-OH is a p-toluenesulfonic acid salt represented by formula (X):

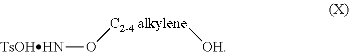

In some embodiments, in step a), the salt of H₂N—O—C₂₋₄ alkylene-OH is a compound represented by formula (K-1):

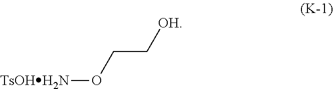

With reference to step a), the silylating agent, the first base, the first solvent, the first mixture, the O-silyl protected compound thereof, and the reaction conditions are each described according to Section (III). In some embodiments, the silylating agent is trimethylsilyl chloride (TMSCl). In some embodiments, the first base is 4-methylmorpholine. In some embodiments, the first mixture is formed in-situ. In some embodiments, the first solvent is tetrahydrofuran (THF).

With reference to step b) via an acid chloride of formula (XII), in some embodiments, step b) is conducted by adding a second mixture including the compound of formula (XII) or the salt thereof and a second solvent to the first mixture of step a) to form the compound of formula (XI) or the salt thereof. The second solvent and reaction conditions are each described according to Section (III). In some embodiments, the second solvent is tetrahydrofuran (THF) or methyl tert-butyl ether (MTBE).

With reference to step b) via an acid of formula (XIII), in some embodiments, step b) is conducted with one or more amide coupling reagents in a solvent to form the compound of formula (XI) or the salt thereof. The one or more amide coupling reagents can be any peptide coupling agents that are capable of activating the —C(O)OH group of formula (XIII) for an amide formation to provide the compound of formula (XI) or the salt thereof. Suitable peptide coupling agents include N,N′-dicyclohexylcarbodiimide (DCC), N,N′-diisopropylcarbodiimide (DIC), 1-[bis(dimethylamino)methylene]-1H-1,2,3-triazolo[4,5-b]pyridinium 3-oxide hexafluorophosphate (HATU), 3-[bis(dimethylamino)methyliumyl]-3H-benzotriazol-1-oxide hexafluorophosphate (HBTU), 1-hydroxy-7-azabenzotriazole (HOAt), hydroxybenzotriazole (HOBt), benzotriazol-1-yloxy)tripyrrolidinophosphonium hexafluorophosphate (PyBOP), and thiocarbonyldiimidazole (TCDI).

In some embodiments, the compound of formula (XI) is selected from the group consisting of:

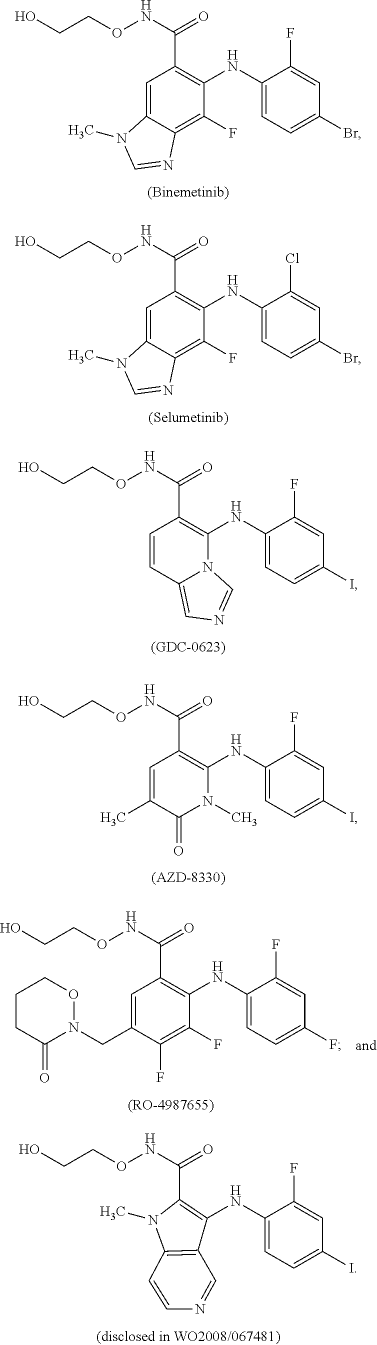 [SMILES: Cn1cnc2c(F)c(Nc3ccc(Br)cc3F)c(C(=O)NOCCO)cc21 ; Cn1cnc2c(F)c(Nc3ccc(Br)cc3Cl)c(C(=O)NOCCO)cc21 ; O=C(NOCCO)c1ccc2cncn2c1Nc1ccc(I)cc1F ; Cc1cc(C(=O)NOCCO)c(Nc2ccc(I)cc2F)n(C)c1=O ; O=C(NOCCO)c1cc(CN2OCCCC2=O)c(F)c(F)c1Nc1ccc(F)cc1F ; Cn1c(C(=O)NOCCO)c(Nc2ccc(I)cc2F)c2cnccc21]

In some embodiments, the compound of formula (XI) is selected from the group consisting of:

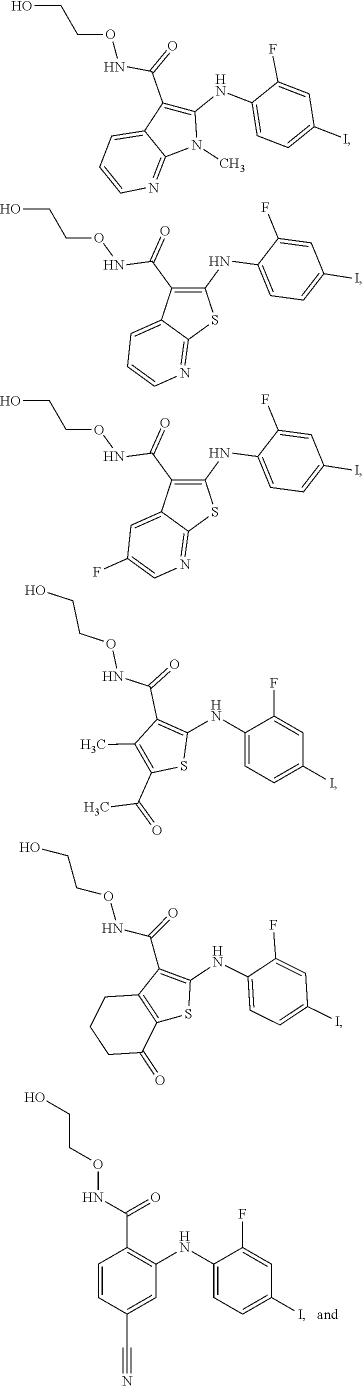

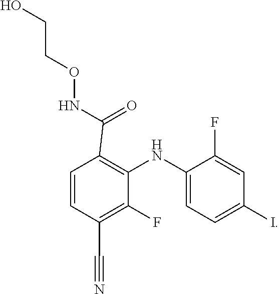 [SMILES: N#Cc1ccc(C(=O)NOCCO)c(Nc2ccc(I)cc2F)c1F]

VI. Compounds

In a sixth aspect, the present disclosure provides a compound represented by formula (X):

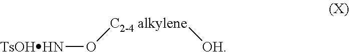

In some embodiments, the C₂₋₄ alkylene is CH₂CH₂ or CH₂CH₂CH₂. In some embodiments, the C₂₋₄ alkylene is CH₂CH₂.

In some embodiments, the compound of formula (X) is represented by formula (K-1):

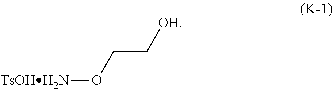

### VI. EXAMPLES

Reagents were purchased from commercial sources and were used as received. ¹H nuclear magnetic resonance spectra were obtained on a Bruker AVANCE 300 spectrometer at 300 MHz or an AVANCE 500 spectrometer at 500 MHz with tetramethylsilane used as an internal reference. ¹³C nuclear magnetic resonance spectra were obtained on a Bruker AVANCE 500 spectrometer at 125 MHz with the solvent peak used as the reference. HPLC analyses were obtained on a Waters Alliance 2695 HPLC with a Waters 2487 Dual Wavelength Detector using the methods below with the detector at the specified wavelength. LCMS analysis was conducted on a Perkin Elmer Sciex API 150EX mass spectrometer connected to a Shimadzu LC-10AD HPLC.

### General Analytical Methods

### UPLC Method for Purity Determination of the Compound of Formula (I)

Column: Acquity UPLC CSH C18, 1.7 μm, 2.1×150 mm
Column Temperature: 55° C.
Autosampler Temperature: 25° C.
Detection: 248 nm
Mobile Phase A: 0.05% Formic acid in water
Mobile Phase B: Acetonitrile
Gradient: see Table below
Flow Rate: 0.3 mL/min
Injection Volume: 1 μL
Injection Mode: Gradient start at injection for H-Class
Data Collection Time: 22 min
Re-equilibration Time: 7 min
Total Analysis Time: 29 min
Needle Wash: Methanol
Seal Wash: Acetonitrile/water, 50:50

| Time | | |
|---|---|---|
| (min) | % A | % B |
| Initial | 90.0 | 10.0 |
| 0.5 | 90.0 | 10.0 |
| 2.0 | 75.0 | 25.0 |
| 20.0 | 10.0 | 90.0 |
| 22.0 | 10.0 | 90.0 |
| 22.5 | 90.0 | 10.0 |

### Chemical Development HPLC Method—TFA

Column: Waters Xbridge C18(2), 3.5 μm, 150×4.6 mm
Detection: 254 nm
Mobile Phase A: 0.05% TFA in water
Mobile Phase B: 0.05% TFA in Acetonitrile
Gradient: see Table below
Flow Rate: 1 mL/min

| Time | | |
|---|---|---|
| min | % A | % B |
| 0.0 | 95.0 | 5.0 |
| 5.0 | 95.0 | 5.0 |
| 23.0 | 5.0 | 95.0 |
| 25.0 | 5.0 | 95.0 |
| 25.1 | 95.0 | 5.0 |
| 30.0 | 95.0 | 5.0 |

### Chemical Development HPLC Method—Formic Acid

Column: Waters Atlantis T3, C18, 3.5 μm, 150×4.6 mm
Detection: 254 nm
Mobile Phase A: 0.05% formic acid in water
Mobile Phase B: 0.05% formic acid in Acetonitrile
Gradient: see Table below
Flow Rate: 0.8 mL/min

| Time | | |
|---|---|---|
| (min) | % A | % B |
| 0.0 | 95.0 | 5.0 |
| 5.0 | 95.0 | 5.0 |
| 15.0 | 5.0 | 95.0 |
| 25.0 | 5.0 | 95.0 |
| 25.1 | 95.0 | 5.0 |
| 30.0 | 95.0 | 5.0 |

### Example 1: Development and Preparation of 2-(2-Hydroxyethoxy)isoindoline-1,3-dione (J)

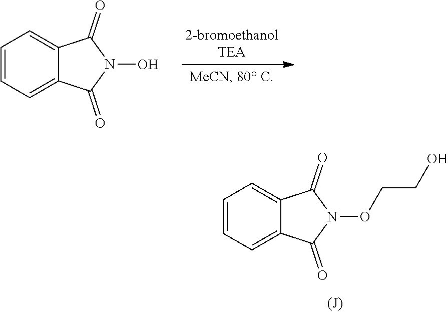 [SMILES: O=C1c2ccccc2C(=O)N1O ; O=C1c2ccccc2C(=O)N1OCCO]

The reaction proceeded well with excess amounts of 2-bromoethanol and trimethylamine. However, the isolation of the compound (J) faced challenges due to excess amounts of reagents present in the reaction mixture. The reaction conditions were explored with the reduced excess amounts of 2-bromoethanol and trimethylamine in the process for the isolation of the compound (J). Since the reaction was performed in just 2.5 volumes of acetonitrile, and initially, all the solids that precipitated during the reaction were trimethylamine hydrobromide. By simply charging 12 volumes of deionized water, the salts first dissolved, and then clean product precipitated from the reaction mixture, with some residual trimethylamine remaining.

Table 1 shows various reaction conditions for the preparation and isolation of 2-(2-hydroxyethoxy)isoindoline-1,3-dione (J).

TABLE 1
Preparation of 2-(2-hydroxyethoxy)isoindoline-1,3-dione (J)

| Entry | Scale | | Compound (J) |
|---|---|---|---|
| No. | (g) | Conditions | (g) (% yield) |
| 1 | 40.0 | 1.0 eq. hydroxyphthalamide | 37.91 g |
| | | 1.4 eq. bromoethanol | (75%) |
| | | 1.1 eq. TEA | |
| | | 2.5 vol MeCN | |
| | | 80° C., 18 h | |
| 2 | 40.0 | 1.0 hydroxyphthalamide | 37.46 g |
| | | 1.4 eq. bromoethanol | (74%) |
| | | 1.1 eq. NMM | |
| | | 2.5 vol MeCN, | |
| | | 80° C., 18 h | |
| 3 | 300.0 | 1.0 eq. hydroxyphthalamide | 190.5 g |
| | | 1.4 eq. bromoethanol | (50%) |
| | | 1.1 eq. TEA | |
| | | 2.5 vol MeCN | |
| | | 80° C., 18 h | |
| 4 | 150.0 | 1.0 eq. hydroxyphthalamide | 98.5 g |
| | | 1.4 eq. bromoethanol | (52%) |
| | | 1.1 eq. TEA | |
| | | 2.5 vol MeCN | |
| | | 80° C., 18 h | |
| 5 | 100.0 | 1.0 eq. hydroxyphthalamide | 49-56% |
| | | 1.4 eq. bromoethanol | |
| | | 1.1 eq. TEA | |
| | | 2.5 vol MeCN | |
| | | 80° C., 20 h | |
| 6 | 50.0 | 1.0 eq. hydroxyphthalamide | 35.1 g |
| | | 1.4 eq. bromoethanol | (55%) |
| | | 1.1 eq. TEA | |
| | | 2.5 vol MeCN | |
| | | 80° C., 18 h | |
| 7 | 1200 | 1.0 eq. hydroxyphthalamide | 765 g |
| | | 1.4 eq. bromoethanol | (50%) |
| | | 1.1 eq. TEA | |
| | | 2.5 vol MeCN | |
| | | 80° C., 18 h | |

Entry 1: shows the formation of the clean product in good yield with 1.4 equivalents of 2-bromoethanol and 1.1 equivalents of trimethylamine followed by the addition of water at the end of reaction (e.g., added 12 vol water and cooled to 0° C.).

Entry 2: As the only impurity remaining at the end of the reaction was the trimethylamine, the reaction was attempted using NMM as the base.

Entry 3: The process conditions were scaled to 300 g of hydroxyphthalimide using the same conditions as Entry 1. Addition of higher amounts of water may have been necessary to precipitate more product.

Entry 5: The compound (J) was crystalline as observed under a microscope. To solve the slow filtration of the compound (J) in the process, an attempt was made to enhance crystal growth and improve the filtration rate. A 100-g batch was prepared and the reaction mixture was divided into four equal portions. The first portion was quenched by addition of water in a single portion similar to the process as described in Entry 3. This gave a slow filtering suspension (approximately 1 h). The yield of this batch was 52%. The addition of water was performed over 30 minutes in the second portion. This also gave a slow filtering suspension and the compound (J) was isolation in 53% yield. The water addition time was increased to three hours in the third portion, but in this case there was no crystallization after stirring overnight, so the batch was seeded, which induced crystallization. This did give an improved filtration rate (5-10 min) and the product was isolated in 49% yield. The final portion was filtered first to remove the trimethylamine hydrobromide salts and then the water was added over three hours. After aging the suspension overnight, the filtration was fast (5-10 min). The yield of the fourth portion was 56%. The methodology of the fourth portion (pre-filtration followed by slow addition of water) was incorporated into the process.

Entry 6: The amount of water was increased to 14 volumes in order to increase the yield. The yield from this batch was 55% which was not significantly improved over the normal 50% yield obtained with 12 volumes of water. However, this process was successful on a demonstration batch run on 1200 g of hydroxyphthalimide.

Entry 7: The demonstration batch was performed on 1200 g of hydroxyphthalimide and produced a 50% yield of the desired product. The trimethylamine HBr salts were filtered prior to adding the water.

### Preparation of 2-(2-hydroxyethoxy)isoindoline-1,3-dione (J) (Entry 7)

To a 30-L, jacketed reactor, inerted with N₂ flow at 1 L/min for 2 h was charged hydroxyphthalamide (1.2 kg, 7.36 mol) and acetonitrile-1 (3 L, 2.5 vol). The stirring was started. Triethylamine (1.128 L, 1.1 eq.) was charged over an hour while maintaining batch temperature 19-20° C. (Note: This addition is exothermic). 2-Bromoethanol (730 mL, 1.4 eq.) was charged over a 25 min period while maintaining the batch temperature at 19° C. (Note: This addition is exothermic). The batch temperature was heated to 70-80° C. and maintained at this temperature for 19 h. In-process HPLC analysis revealed that there was less than 5% starting material relative to compound (J). The batch was cooled to 20.6° C. over a period of an hour. The batch was filtered to remove trimethylamine hydrobromide salt. [Note: Approximately 20% of the salt remained at the bottom of the reactor which was removed after rinsing with mother liquor (≈400 mL)]. Acetonitrile (600 mL, 0.5 vol) was charged to the reactor and then to the filter cake as a wash. The filtrate was charged back to the reactor and DI-water (17 L, 14 vol) was added over a period of 2 h by a dosing pump while maintaining the batch temperature at 20±5° C. with an agitation speed 130 revolutions/min. The agitation speed was reduced to 115 after 3 h when the white precipitate started to appear. The slurry was agitated at this temperature (20° C.) for 16 h. The batch was cooled to 14° C. and filtered in an 18-inch, Nutsche equipped with a polypropylene cloth filter. The reactor and filter cake was rinsed with DI-water (2.5 L). The wet cake was conditioned two days on the filter under nitrogen. The wet cake (1502 g) was dried in a vacuum oven at 40-50° C. to afford 765 g (50% yield). The ¹H NMR analysis of the product was consistent with the assigned structure. KF analysis: 0.37% water.

### Example 2: Phthalimide Deprotection of 2-(2-Hydroxyethoxy)isoindoline-1,3-dione (J)

Any alternative reagent utilized in the deprotection reaction needed to produce an insoluble byproduct that could be removed by filtration since the compound of formula (K) cannot be isolated through distillation due to decomposition. With this in mind, a small screen was performed using ethylenediamine, ethanolamine, and cyclohexyldiamine (as a mixture of cis and trans isomers) for the deprotection, but only the ethylenediamine reaction produced a precipitate (see Entries 1-3 of Table 2). The deprotection reaction was repeated with heating to attempt to drive to completion the cyclization and the release of the final product (see Entry 4 of Table 2). ¹H NMR analysis revealed the product was contaminated with partially deprotected intermediate, as well as methanol and ethylenediamine. Since this reaction would likely be difficult to perform with stoichiometric ethylylenediamine, and since residual ethylenediamine would likely be highly detrimental in step 6) of the process for preparing the compound of formula (I), a more volatile deprotection reagent was sought.

Further work using hydrazine as the deprotection reagent was also conducted. It was attempt to make a THF solution of the 2-(aminooxy)ethanol rather than an isolated oil in order to minimize or eliminate distillation of the final product. In the first experiment (see Entry 5 of Table 2) with hydrazine, the reaction stalled with 8% residual compound (J) remaining. This material was subject to a second hydrazine deprotection reaction, which caused the reaction to go to completion. This material was isolated by multiple chloroform treatments and evaporation to dryness. In Entry 6, once the reaction was complete, it was directly solvent exchanged into THF. However, large amounts of methanol remained after two distillations. Also less than 1% of the phthalimide byproduct remained. The deprotection with hydrazine was not further pursued.

TABLE 2
Phthalimide Deprotection of 2-(2-Hydroxyethoxy)isoindoline-
1,3-dione (J)

| | | | Compound (K) |
|---|---|---|---|
| Entry No. | Scale (g) | Conditions | (g) (% yield) |
| 1 | 0.5 | 1.0 eq. compound (J) | — |
| | | 2.0 eq. ethylenediamine | |
| | | 10 vol MeOH | |
| | | 5 days, rt | |
| 2 | 0.5 | 1.0 eq. compound (J) | — |
| | | 2.0 eq. ethanolamine | |
| | | 10 vol MeOH | |
| | | 5 days, rt | |
| 3 | 0.5 | 1.0 eq. compound (J) | — |
| | | 2.0 eq. cyclohexyldiamine | |
| | | 10 vol MeOH | |
| | | 5 days, rt | |
| 4 | 3.0 | 1.0 eq. compound (J) | — |
| | | 2.0 eq. ethylenediamine | |
| | | 10 vol MeOH | |
| | | 8 h at 60° C., 24 h at rt | |
| 5 | 30.0 | 1.0 eq. compound (J) | 8.07 |
| | | 1.0 eq. hydrazine monohydrate | (72%) |
| | | 12 vol MeOH | |
| | | 65° C., 2 h. | |
| 6 | 31.5 | 1.0 eq. compound (J) | Not |
| | | 1.0 eq. hydrazine monohydrate | determined |
| | | 12 vol MeOH | |
| | | 65° C., 2 h. | |
| 7 | 100.0 | 1.0 eq. compound (J) | 26.6 g |
| | | 1.25 eq. hydrazine monohydrate | (71%) |
| | | 12 vol MeOH | |
| | | 65° C., 2 h. | |

### Example 3: Phthalimide Deprotection with Ammonia to Form 2-(aminooxy)ethanol (K)

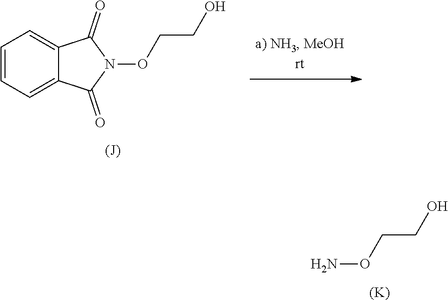

A number of development runs were performed to establish ammonia as the reagent for this deprotection as shown in Table 3. Ammonia was considered for use as the deprotecting agent since this is cheap, easily removed, and sold in varying concentrations in methanol.

TABLE 3
Phthalimide Deprotection With Ammonia

| | | | Compound (K) |
|---|---|---|---|
| Entry No. | Scale (g) | Conditions | (g) (% yield) |
| 1 | 1.0 | 1.0 eq. compound (J) | 0.5 |
| | | 10 vol 7N NH₃—MeOH | (>100%) |
| | | 70° C., 45 min | |
| 2 | 1.0 | 1.0 eq. compound (J) | 0.6 |
| | | 10 vol 7N NH₃—MeOH | (>100%) |
| | | RT, 22 h | |
| 3 | 1.0 | 1.0 eq. compound (J) | 0.4 |
| | | 10 vol 7N NH₃—MeOH | (>100%) |
| | | 45° C., 2 h | |
| 4 | 1.0 | 1.0 eq. compound (J) | 0.2 |
| | | 10 vol 1.8N NH₃—MeOH | (54%) |
| | | RT, 20 h | |
| 5 | 1.0 | 1.0 eq. compound (J) | 0.3 |
| | | 10 vol 3.5N NH₃—MeOH | (80%) |
| | | RT, 17 h | |
| 6 | 1.0 | 1.0 eq. compound (J) | 0.35 |
| | | 10 vol 7N NH₃—MeOH | (94%) |
| | | RT, 2 h | |
| 7 | 1.0 | 1.0 eq. compound (J) | 0.24 |
| | | 10 vol 3.5N NH₃—MeOH | (64%) |
| | | RT, 3 h | |
| 8 | 1.0 | 1.0 eq. compound (J) | 0.22 |
| | | 10 vol 7N NH₃—MeOH | (59%) |
| | | RT, 1 h | |
| 9 | 1.0 | 1.0 eq. compound (J) | 0.28 |
| | | 10 vol 3.5 N NH₃—MeOH | (75%) |
| | | RT, 2 h | |
| 10 | 1.0 | 1.0 eq. compound (J) | 0.26 |
| | | 10 vol 7N NH₃—MeOH | (70%) |
| | | RT, 1 h 15 min | |
| 11 | 40.0 | 1.0 eq. compound (J) | 12.2 |
| | | 10 vol 7N NH₃—MeOH | (83%) |
| | | RT, 1.5 h | |
| 12 | 40.0 | 1.0 eq. compound (J) | 11.8 |
| | | 10 vol 3.5N NH₃—MeOH | (79%) |
| | | RT, 4 h | |

Initial efforts revealed that the phthalimide byproduct was indeed insoluble in the methanol solvent.

Entry 1: Because the ammonia may be escaping the reaction heated in an open reactor, the reaction was carried out in a sealed tube at 70° C. No starting material was observed. The desired product was observed along with several impurities. The third reaction was also carried out in a sealed tube. The desired product was cleanly isolated after filtering the cold batch (0-5° C.) to remove the phthalimide byproduct, washing with chloroform, and concentrating (without distillation). The ¹H NMR of Entry 1 indicated a clean deprotected product of (K).

Entries 2-6: The next several development runs investigated varying temperature and ammonia concentration in the deprotection reaction. This sealed tube reaction was run with 7 N ammonia solution at room temperature rather than elevated temperature (Entry 2). This produced the desired product. The reaction was again attempted in a sealed tube with 7 N ammonia solution at 45° C. (Entry 3). When the concentration of ammonia was dropped to 1.8 N and the reaction run at room temperature, the reaction did not go to completion (Entry 4). The ammonia concentration was increased to 3.5 N in the next experiment (Entry 5). All the starting material was consumed and the desired product was observed. Going back to 7 N ammonia solution at room temperature in Entry 6, the deprotection went to completion. These experiments established that the deprotection could be completed at room temperature with a minimum of 3.5 N ammonia solution.

Entries 7-10: The next four experiments compared running with either 3.5 N or 7.0 N ammonia solution in an autoclave versus an reactor at atmospheric pressure. There was no difference seen between 3.5 N and 7.0 N and using an autoclave or not.

Entries 11-12: The reaction was scaled successfully to 40 g using 7.0 N ammonia solution in Entry 11. The 40 g reaction produced 2-(aminooxy) ethanol in 83% yield. Diluted ammonia solution (3.5 N) showed equal performance on 40 g scale (79% yield, Entry 12). Therefore, the reaction was scaled using 3.5 N ammonia solution in a normal reactor at atmospheric pressure.

### Example 4: Development and Preparation of p-Toluenesulfonic acid salt of 2-(aminooxy)ethanol (K-1)

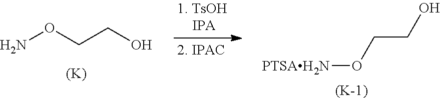

The product from 1:1 isopropanol/isopropyl acetate (see Entry 1 of Table 4) was isolated as a brilliant white solid (30.2 g, 62%). The ¹H NMR, indicated it was a very pure form of compound (K-1).

TABLE 4
p-Toluenesulfonic acid salt (K-1) Formation

| | | | Compound (K-1) |
|---|---|---|---|
| Entry No. | Scale (g) | Conditions | (g) (% yield) |
| 1 | 15.0 | 1.0 eq. compound (K) | 30.2 |
| | | 1.0 eq. tosylic acid | (62%) |
| | | 20 vol IPA | |
| | | 20 vol IPAC | |
| | | 40° C.-rt | |

### Example 5: Preparation of p-Toluenesulfonic acid salt of 2-(aminooxy)ethanol (K-1) Step 8a) Step 8b)

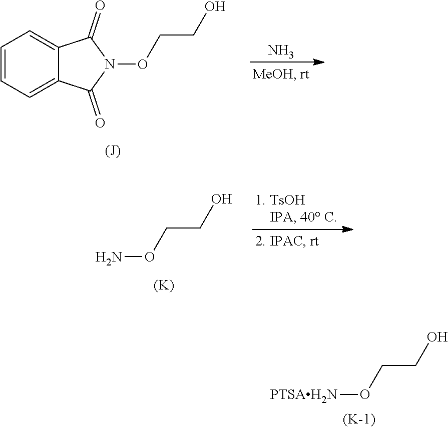

To eliminate the use of chloroform in the purification of the compound (K) as the free base, a telescoped approach was used to combine the salt formation with the ammonia mediated phthalimide deprotection. A 15-g pilot reaction was performed to test the telescoped procedure (see Entry 1 of Table 5). After the deprotection reaction with 3.5 N ammonia solution, the phthalimide byproduct was removed by filtration. The methanol filtrate was solvent exchanged to isopropanol. To this solution was added one equivalent of pTSA dissolved in isopropanol at 40° C. To induce crystallization of the salt, five volumes of isopropyl acetate was added to the batch. The resulting slurry was filtered and dried to afford a 60% yield of the desired pTSA salt. The demonstration batch of the telescoped deprotection was completed on 700 g of compound (J) to afford the desired p-toluenesulfonic acid salt (K-1) in a 50% yield.

TABLE 5
p-Toluenesulfonic acid salt (K-1)
Formation by Telescoped Approach

| Entry | Scale | | Compound (K-1) |
|---|---|---|---|
| No. | (g) | Conditions | (g) (% yield) |
| 1 | 15.0 | Step 8a): 1.0 eq. compound (J); | 10.8 |
| | | 3.5 N NH₃—MeOH (10 vol); RT | (60%) |
| | | Step 8b): 1.0 eq. tosylic acid | |
| | | in IPA addition at 40° C.; | |
| | | and 5 vol IPAC at rt | |
| 2 | 700 | Step 8a): 1.0 eq. compound (J); | 422.1 |
| | | 3.5 N NH₃—MeOH (10 vol); RT | (50%) |
| | | Step 8b): 1.0 eq. tosylic acid | |
| | | in IPA addition at 40° C.; | |
| | | and 5 vol IPAC at rt | |

### Preparation of p-Toluenesulfonic acid salt of 2-(aminooxy)ethanol (K-1) (Entry 2)

To a 10-L, jacketed reactor, inerted with N₂ flow (3 L/min) for 10 min was charged with compound (J) (700 g), methanol (3.5 L, 5 vol), and stirring was started. A 2 N HCl scrubber was set up and connected to the vent of the reactor. Methanolic ammonia solution (7 N, 3.5 L, 5 vol) was charged over a 15 min period while maintaining the batch temperature less than 30° C. (Note: This addition is exothermic). The batch was stirred at ambient (20-25° C.) for 17 h. In-process NMR analysis revealed there was less than 5% compound (J) relative to compound (K). The batch was filtered to remove the white phthalimide impurity. (Note: The pump exhaust was vented through an HCl scrubber.) The reactor and filter cake was rinsed with isopropyl alcohol (IPA) (350 mL, 0.5 vol). The filtrate was charged back to the reactor. The batch was vacuum distilled under reduced pressure to 5 vol (3.5 L). IPA (3.5 L, 5 vol) was charged and the batch was distilled under reduced pressure while maintaining the batch the temperature below 50° C. to 5 vol (3.5 L). ¹H NMR analysis showed that 2.9% MeOH was present. The batch was filtered to remove a second crop of white phthalimide impurity and the solids were washed with IPA (1.4 L, 2 vol). The combined filtrate and wash were charged back to the reactor and the batch temperature was brought to 40=5° C. A pTSA solution was prepared using p-toluenesulfonic acid monohydrate (646 g) and IPA (1.4 L, 2 vol). The pTSA solution was charged over a 40 min period while maintaining batch temperature at 40±5° C. Isopropyl acetate (IPAc) (3.5 L, 5 vol) was charged over 10 min. The batch was cooled at 15 5° C. The desired product began to crystallize at 20° C. The batch was stirred for 5 h and was then filtered through Whatman filter paper. IPAc (1.4 L, 2 vol) was charged to the reactor and the rinse was passed over the collected solids. The batch was conditioned until liquid stopped eluting and the wet cake was dried in a vacuum oven at 40-50° C. The final net weight was 422.1 g (50% yield). The ¹H NMR analysis was consistent with the assigned structure. KF analysis: 0.18% water.

### Example 6: Optimization and Preparation of 2-(2-Hydroxyethoxy)isoindoline-1,3-dione (J)

The alkylation reactions of N-hydroxy-phthalimide with 2-bromoethanol were conducted under various conditions:

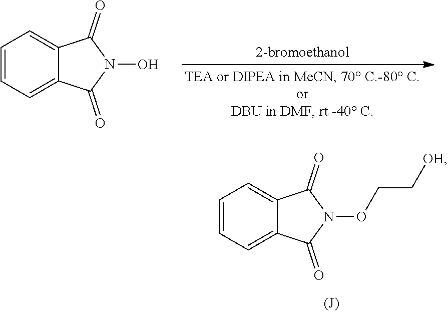 [SMILES: O=C1c2ccccc2C(=O)N1O ; O=C1c2ccccc2C(=O)N1OCCO]

and the results are summarized in Table 6 below.

TABLE 6
Various Conditions for Forming 2-(2-Hydroxyethoxy)isoindoline-1,3-dione (J)

| Entry | Scale | | |
|---|---|---|---|
| No. | (g) | Conditions | Compound (J) |
| 1 | 15.0 g | 1.0 eq. hydroxyphthalamide | Yield: 59.5% (3.78 g) |
| | | 1.4 eq. bromoethanol | Purity: 88.6 area % |
| | | 1.1 eq. TEA | Isolation: EA extraction followed by EA/n- |
| | | 2.5 vol MeCN | heptane (1:1) trituration |
| | | 78° C., overnight | |
| 2 | 20.0 | 1.0 eq. hydroxyphthalamide | Yield: 78% (19.8 g) |
| | | 1.4 eq. bromoethanol | Purity: 81.2 area % |
| | | 1.2 eq. TEA | Isolation: EA extraction followed by n- |
| | | 2.5 vol MeCN | heptane trituration |
| | | 60° C., overnight | |
| 3 | 20.0 | 1.0 eq. hydroxyphthalamide | Yield: 54.0% (13.74 g) |
| | | 1.2 eq. bromoethanol | Purity: 91.6 area % |
| | | 1.2 eq. TEA | Isolation: solvent exchange with IPA |
| | | 2.5 vol MeCN | followed by concentration |
| | | 78° C., overnight | |
| 4 | 5.0 | 1.0 eq. hydroxyphthalamide | Yield: 81.2% (5.2 g) |
| | | 1.2 eq. bromoethanol | Purity: 88.7 area % |
| | | 1.2 eq. DIPEA | Isolation: EA extraction followed by n- |
| | | 2.5 vol MeCN | heptane trituration |
| | | 78° C., overnight | |
| 5 | 20.0 | 1.0 eq. hydroxyphthalamide | Yield: 73.0% (18.55 g) |
| | | 1.2 eq. bromoethanol | Purity: 85.7 area % |
| | | 1.2 eq. DIPEA | Potency by 1HNMR: 89.91 wt % |
| | | 2.5 vol MeCN | Isolation: EA extraction followed by n- |
| | | 78° C., overnight | heptane trituration |
| 6 | 20.0 | 1.0 eq. hydroxyphthalamide | Yield: 71.6% (18.18 g, white solid) |
| | | 1.2 eq. bromoethanol | Purity: 87.8 area % |
| | | 1.2 eq. DIPEA | |
| | | 2.5 vol MeCN | |
| | | 70° C., overnight | |
| 7 | 5.0 | 1.0 eq. hydroxyphthalamide | Yield: 43.1% (2.74 g) |
| | | 1.1 eq. bromoethanol | Purity: 97.2 area % |
| | | 1.1 eq. DBU (1.0 + 0.1) | Additional material from concentrating |
| | | 2.5 vol DMF | the filtrate to dryness: |
| | | rt, overnight | 1.79 g in a purity of 86.0 area % |
| 8 | 5.0 | 1.0 eq. hydroxyphthalamide | Yield: 56.7% (3.6 g, white solid) |
| | | 1.1 eq. bromoethanol | Purity: 89.4 area % |
| | | 1.0 eq. DBU | |
| | | 2.5 vol DMF | |
| | | 40° C., overnight | |
| 9 | 10.0 | 1.0 eq. hydroxyphthalamide | Yield: 44.2% (5.62 g, white solid) |
| | | 1.1 eq. bromoethanol | Purity: 87.6 area % |
| | | 1.1 eq. DBU | |
| | | 2.5 vol DMF | |
| | | rt, overnight | |
| 10 | 5.0 | 1.0 eq. hydroxyphthalamide | Yield: 52.0% (3.3 g, white solid) |
| | | 1.1 eq. bromoethanol | Purity: 87.6 area % |
| | | 1.1 eq. DBU | |
| | | 2.5 vol DMF | |
| | | rt, overnight | |
| 11 | 20.0 | 1.0 eq. hydroxyphthalamide | Yield: 45.6% (11.58 g, white solid) |
| | | 1.2 eq. bromoethanol | Purity: 95.0 area % |
| | | 1.2 eq. DBU | Additional material from concentrating |
| | | 2.5 vol DMF | the filtrate to dryness: |
| | | rt, overnight | 7.7 g in a purity of 71.4 area % |
| 12 | 5.0 | 1.0 eq. hydroxyphthalamide | Yield: 72.4% (9.2 g, white solid) |
| | | 1.2 eq. bromoethanol | Purity: 89.0 area % |
| | | 1.2 eq. DBU | NMR potency: 92 wt % |
| | | 2.5 vol DMF | Isolation: Precipitation was conducted |
| | | rt, overnight | with 4 vol EA and 10 vol n-heptane. |
| | | | Yield: 75.1% (9.54 g, white solid) |
| | | | Purity: 87.6 area % |
| | | | Isolation: Precipitation was conducted |
| | | | with 4 vol EA and 15 vol n-heptane. |

Entry 1: Reaction conversion was 85%, with product IPC purity 77.11 area %. Reaction was filtered and rinsed filter cake with 7.5 mL (0.5 vol) of acetonitrile. Filtrate was divided into three portions by weight. Isolation-1 by water (70 mL, 14 vol.) trituration: 1.7 g (purity 89.3%, yield 26.8%), HPLC showed 50% product was left in filtrate (pH=4). Additional ethyl acetate extraction of the filtrate recovered 1.79 g product with 62.4% purity. Isolation-2 by ethyl acetate extraction: 6.14 g (purity 77.0%, crude recovery 96.7%). After ethyl acetate/heptane (1:1) trituration, 3.78 g product was isolated (purity 88.6%, yield 59.5%). Isolation-3 by isopropyl alcohol (IPA) trituration (70 mL, 14 vol.): small amount of solid crystallized out (not isolated).

Entry 2: 2-bromoethanol was slowly dosed into the hot solution of hydroxyphthalamide and TEA in acetonitrile at 60° C. and addition of 2-bromoethanol took 30 min. 0.2 eq. TEA and 0.1 eq. 2-bromoethanol was added on the 2nd day and heated for another 4 hr at 60° C. No significant exothermic effect was observed during addition of bromoethanol, highest reaction temperature reached 63° C. Reaction conversion was 83% with product IPC purity of 72.0% after overnight heating at 60° C. and went to 95% conversion with product IPC purity 83.1% after adding more TEA and 2-bromoethanol the next day. Work-up: ethyl acetate (EA) extraction followed by n-heptane trituration (4 vol of EA and 5 vol of n-heptane for trituration). 19.8 g product was isolated (purity 81.2%, yield 78%).

Entry 3: 2-bromoethanol was slowly dosed into the hot solution of hydroxyphthalamide and TEA in acetonitrile at 78° C. and addition of 2-bromoethanol took 55 min. 0.1 eq. TEA and 0.1 eq. 2-bromoethanol was added on the 2nd day and heated for another 2 hr at 78° C. No significant exothermic effect was observed during addition of bromoethanol. Reaction conversion was 85% with product IPC purity of 69.1% after overnight heating at 78° C. and went to 95% conversion with product IPC purity of 77.3% after adding more TEA and 2-bromoethanol the next day. Lower yield was due to a different work-up method. Work-up: solvent exchange with IPA (5 vol). Filtrate was concentrated to got 19 g light yellow solid with 50% product according to HPLC.

Entry 4: Reaction conversion was 98% and product IPC purity was 78.4%. Work-up: EA extraction followed by n-heptane trituration. Aqueous layer (4 vol of water) was extracted with EA three times (4 vol, 4 vol, 2 vol).

Entry 5: 2-bromoethanol was slowly dosed into the hot solution of hydroxyphthalamide and DIPEA in acetonitrile at 75° C. and addition of 2-bromoethanol took 25 min. 0.1 eq. DIPEA and 0.1 eq. 2-bromoethanol was added on the 2nd day and heated for another 2 hr at 75° C. Reaction conversion was 95% with product IPC purity of 79.3% after overnight heating at 75° C. and went to >98% conversion with product IPC purity of 83.1% after adding more DIPEA and 2-bromoethanol the next day. Work-up: EA extraction followed by n-heptane trituration. Aqueous layer (4 vol of water) was extracted with EA two times (4 vol×2).

Entry 6: 2-bromoethanol was slowly dosed into the hot solution of hydroxyphthalamide and DIPEA in acetonitrile at 70° C. and addition of 2-bromoethanol took 1 hr. Reaction conversion was 97% with product IPC purity of 85.2 area %.

Entry 7: DBU was added dropwise into a solution of hydroxyphthalamide and 2-bromoethanol in DMF at rt. Addition of DBU took 20 min. Reaction went to 92% conversion after overnight stirring at rt. The next day, 0.1 eq DBU was added and kept it stirring at rt for 3 hr. Reaction went to 96% conversion and product IPC purity was 90.2%.

Entry 8: 2-bromoethanol was slowly dosed into a hot solution of hydroxyphthalamide and DBU in DMF at 40° C. and addition of 2-bromoethanol took 20 min. Reaction went to 90% conversion after overnight heating at 40° C. and product IPC purity was 85.6%.

Entry 9: DBU was slowly dosed into a solution of hydroxyphthalamide and 2-bromoethanol in DMF at rt and addition of DBU took 20 min. Addition of DBU was exothermic and highest reaction temperature reached 53° C. during the addition. Reaction went to 95% conversion and product IPC purity was 89.2 area % after overnight stirring. 0.1 eq of 2-bromoethanol was added the next day, but reaction conversion did not change after 2 hr. Then 0.1 eq of DBU was added. Reaction conversion went to 97% 2 hr later and product IPC purity was 88.8%. Work-up: Solid was obtained by directly concentrating organic layer (10 vol of EA) to dryness after three water wash (4 vol×3) and without purification to check material recovery.

Entry 10: 2-bromoethanol was slowly dosed into a solution of hydroxyphthalamide and DBU in DMF at rt and addition of 2-bromoethanol took 20 min. Addition of 2-bromoethanol was exothermic and highest reaction temperature reached 28° C. during the addition. Reaction went to 96% conversion and product IPC purity was 90.8 area %. Work-up: Solid was obtained by directly concentrating organic layer (10 vol of EA) to dryness after two water wash (4 vol×2) and without purification to check material recovery.

Entry 11: 2-bromoethanol was slowly dosed into a solution of hydroxyphthalamide and DBU in DMF at rt and addition of 2-bromoethanol took 1 hr. Addition of 2-bromoethanol was exothermic and highest reaction temp. reached 31° C. Reaction went to 97% conversion after overnight stirring and product IPC purity was 87.6 area %.

Entry 12: Reaction was set up in the same way as Entry 11. Addition of 2-bromoethanol was exothermic and highest reaction temperature reached 31° C. Reaction went to 98% conversion and product IPC purity was 86.9 area %. Work-up: Organic layer obtained after extractive work-up was concentrated down to 80 mL (4 vol) and divided into two equal portions by weight. Portion-1: Precipitation was conducted with 4 vol EA and 10 vol n-heptane. Portion-2: Precipitation was conducted with 4 vol EA and 15 vol n-heptane.

### Example 7: Development of Amide Formation of Steps 6a) and 6b)

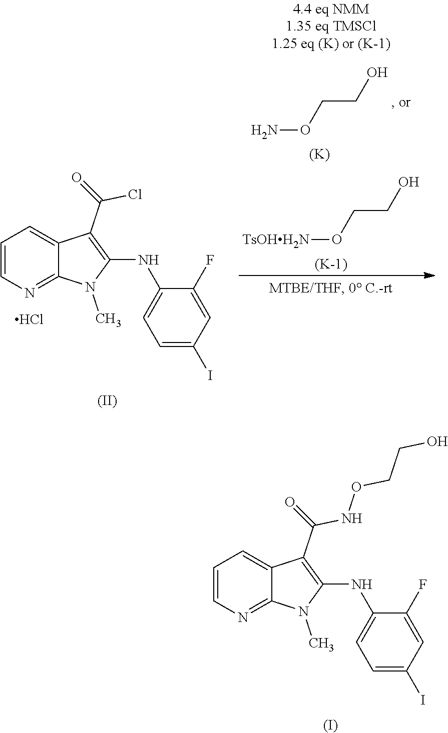

The development of the amide formation of Steps 6a) and 6b) is summarized and shown in Table 7 and Table 8. The primary objectives for the development were as follows:
Identify a suitable solvent for the compound of formula (II) to avoid its decomposition;
Optimize the charcoal treatment to control impurities or develop robust reaction conditions that control impurities without the need for a charcoal treatment;
Incorporate the use of the pTSA salt of the 2-(aminooxy)ethanol; and
Develop a robust crystallization that provides compound (I) in both a high yield and a high purity.

### Identify a Suitable Solvent for the Compound of Formula (II)

Decomposition of compound (II) was found to occur when the material is suspended in THF for a period of time (e.g., >10 h). Therefore, the first course of action was to find a suitable solvent in which to suspend compound (II) for the addition to the first mixture of step 6a). The results of the solvent screen are presented in Table 7.

TABLE 7
Stability of Compound (II) in Various Solvents

| | | Area % | Area % | Area % |
|---|---|---|---|---|
| Entry | Solvent | t = 10 min | t = 2 h | t = 17 h |
| 1 | MTBE | 92.9 | *81.6 | 87.2 |
| 2 | THF | 90.0 | 83.7 | 63.7 |
| 3 | 2-Me-THF | 88.0 | 77.8 | 75.3 |
| 4 | MeCN | 87.4 | 79.2 | 65.5 |
| 5 | IPAc | *81.6 | 93.3 | 90.4 |
| 6 | DCM | 91.4 | 90.5 | 85.3 |
| 7 | Heptane | *68.6 | 88.4 | 91.8 |

*HPLC sample was run too dilute and the value is not representative.

From the slurry experiment, the three solvents that showed the least amount of decomposition were MTBE, isopropyl acetate (IPAc), and heptanes.

The next step investigated was trying to slurry feed compound (II) with various solvents and see the impact on the formation of the compound of formula (I) (see Table 8). In all four entries, the compound (II) starting material was 79.10 pure by HPLC. Comparing the results of THF with MTBE, IPAc, and heptanes, there was only a slight preference for MTBE based on the purity of the compound of formula (I) formed. Ultimately, MTBE was chosen for further development based on these results.

TABLE 8
Amide Formation of Steps 6a) and 6b)

| | | | Compound (I) |
|---|---|---|---|
| Entry No. | Scale (g) | Conditions | (HPLC Area %) |
| 1 | 1.0 g | 1.25 eq. 2-(aminooxy)ethanol | 70.2% |
| | | 1.35 eq. TMSCl | |
| | | 3.4 eq. NMM | |
| | | 5 vol THF | |
| | | 10 vol MTBE | |
| | | 0° C.-rt, 30 min | |
| 2 | 1.0 g | 1.25 eq. 2-(aminooxy)ethanol | 72.5% |
| | | 1.35 eq. TMSCl | |
| | | 3.4 eq. NMM | |
| | | 5 vol THF | |
| | | 10 vol THF | |
| | | 0° C.-rt, 30 min | |
| 3 | 1.0 g | 1.25 eq. 2-(aminooxy)ethanol | 69.4% |
| | | 1.35 eq. TMSCl | |
| | | 3.4 eq. NMM | |
| | | 5 vol THF | |
| | | 10 vol heptanes, | |
| | | 0° C.-rt, 30 min | |
| 4 | 1.0 g | 1.25 eq. 2-(aminooxy)ethanol | 67.0% |
| | | 1.35 eq. TMSCl | |
| | | 3.4 eq. NMM | |
| | | 5 vol THF | |
| | | 10 vol IPAC, | |
| | | 0° C.-rt, 30 min | |

Entries 3 and 4: After 74 h, an additional aliquot was removed and the HPLC area % of Compound (I) was 70.6% and 70.7%, respectively.

### Optimize the Charcoal Treatment to Control Impurities

Some processes for preparing the compound of formula (I) used a 50 wt % body charge of Darco G-60 carbon to treat the batch and remove impurities. This primarily was to remove the late-eluting dimer impurity (RRT 1.92). The amount of Darco G-60 needed for this treatment under the standard THF conditions was investigated in Table 9. Reaction conditions: 1.25 eq. 2-(aminooxy)ethanol, 1.35 eq. TMSCl, 3.4 eq. NMM, 5 vol THF, 10 vol THF, 0-50 wt % Norit Darco G60, 0° C.-rt, 30 min, then 1.5 h stir with charcoal. The loading of Darco G60 charcoal revealed a trend that increased charcoal loading led to decreased amount of dimer impurity. A loading of 50 wt % was needed to reduce the dimer impurity by half in this particular experiment.

TABLE 9
Impurity at RRT 1.92 vs. Charcoal Loading

| Entry No. | Charcoal (wt %) | RRT 1.92 Area % |
|---|---|---|
| 1 | 0% | 0.89 |
| 2 | 10% | 0.90 |
| 3 | 20% | 0.84 |
| 4 | 30% | 0.59 |
| 5 | 40% | 0.69 |
| 6 | 50% | 0.46 |

### Development of the Amide Formation

The next several experiments compared the use of triethylsilyl chloride (TESCl) versus trimethylsilyl chloride (TMSCl) in the coupling reaction. The first experiment with TESCl (see Entry 1 of Table 10) using IPAc as the solvent to slurry compound (II) produced. The impurity profile by Entry 1 with TESCl was similar to reactions run with TMSCl. The remaining reactions in Table 10 compared using TESCl or TMSCl and IPAc or MTBE as the slurry solvent for compound (II). The reactions were quenched with water after complete conversion, and then the biphasic mixture was solvent exchanged to either water/IPAc or water/MTBE mixtures.

TABLE 10
Amide Formation of Steps 6a) and 6b)

| | | | Compound (I) |
|---|---|---|---|
| Entry No. | Scale (g) | Conditions | (% yield) |
| 1 | 1.0 | 1.25 eq. 2-(aminooxy)ethanol | — |
| | | 1.35 eq. TESCl | |
| | | 3.4 eq. NMM | |
| | | 5 vol THF | |
| | | 10 vol IPAc | |
| | | 0° C.-rt, 30 min | |
| 2 | 15.0 | 1.25 eq. 2-(aminooxy)ethanol | HPLC IPC: |
| | | 1.35 eq. TMSCl | 69.6 area % |
| | | 3.4 eq. NMM | |
| | | 5 vol THF | |
| | | 10 vol IPAc | |
| | | 0° C.-rt, 30 min | |
| 3 | 10.0 | 1.25 eq. 2-(aminooxy)ethanol | HPLC IPC: |
| | | 1.35 eq. TESCl | 69.6 area % |
| | | 3.4 eq. NMM | |
| | | 5 vol THF | |
| | | 15 vol MTBE | |
| | | 0° C.-rt, 30 min | |
| 4 | 10.0 | 1.25 eq. 2-(aminooxy)ethanol | HPLC IPC: |
| | | 1.35 eq. TMSCl (slow | 67.5 area %; |
| | | addition over 2 h) | |
| | | 3.4 eq. NMM | Isolated: |
| | | 5 vol THF | 41% (4.1 g) |
| | | 15 vol MTBE | HPLC Final: |
| | | 0° C.-rt, 30 min | 96.9 area %. |

### Development of the Amide Formation Via a Salt Form of 2-(Aminooxy)Ethanol

The development of the amide coupling step using salts of 2-(aminooxy)ethanol was investigated as shown in Table 11. For comparison, the conditions used in Example 13 are included in Entry 7 in the table to illustrate the yield and purity that can be obtained (54% yield, 99.3 area %).

Entry 2: The first experiment with 2-(aminooxy)ethanol pTSA salt (K-1) showed that the salt dissolved readily in the THF/NMM reaction mixture. Addition of compound (II) in 15 volumes of MTBE rapidly (e.g., added in 1 min.) gave a reaction profile with 89 area % compound (I). The major impurities observed in this reaction were the cyclized impurity (RRT 0.97) at 3.4%, and two late eluting impurities at RRT 2.02 (1.2%) and RRT 2.26 (1.10%). There was no dimer impurity made in this reaction (RRT 1.92).

TABLE 11
Amide Formation of Steps 6a) and 6b) via 2-(Aminooxy)ethanol Salt

| Entry | Scale | | Compound (I) | |
|---|---|---|---|---|
| No. | (g) | Conditions | (% yield) | Notes |
| 1 | 1.0 | 1.25 eq. 2-(aminooxy)ethanol sulfate | 48.4% | — |
| | | 1.35 eq. TMSCl | | |
| | | 4.4 eq. NMM | | |
| | | 5 vol THF | | |
| | | 10 vol THF | | |
| | | 0° C.-rt, 30 min | | |
| 2 | 0.5 | 1.25 eq. 2-(aminooxy)ethanol•pTSA (K-1) | HPLC IPC: | a |
| | | 1.35 eq. TMSCl | 89.2 area % | |
| | | 4.4 eq. NMM | | |
| | | 5 vol THF | | |
| | | 15 vol MTBE | | |
| | | 0° C.-rt, 30 min | | |
| 3 | 5.0 | 1.25 eq. 2-(aminooxy)ethanol•pTSA (K-1) | HPLC IPC: | b |
| | | 1.35 eq. TMSCl (slow addition) | 87.0 area % | |
| | | 4.4 eq. NMM | HPLC final | |
| | | 5 vol THF | solid: 96.4 | |
| | | 15 vol MTBE | area % | |
| | | 0° C.-rt, 30 min | | |
| 4 | 12.0 | 1.25 eq. 2-(aminooxy)ethanol•pTSA (K-1) | HPLC IPC: | c |
| | | 1.35 eq. TMSCl (slow addition) | 86.3 area % | |
| | | 4.4 eq. NMM | HPLC final | |
| | | 5 vol THF | solid: 96.6 | |
| | | 15 vol MTBE | area % | |
| | | 0° C.-rt, 30 min | | |
| 5 | 8.0 | 1.25 eq. 2-(aminooxy)ethanol•pTSA (K-1) | Isolated: | d |
| | | 1.35 eq. TMSCl | 42% (3.35 g) | |
| | | 4.4 eq. NMM | HPLC final | |
| | | 5 vol THF | solid: 99.4 | |
| | | 10 vol MTBE | area % | |
| | | 0° C.-rt, 30 min | | |
| 6 | 25.0 | 1.25 eq. 2-(aminooxy)ethanol•pTSA (K-1) | Isolated yield: | e |
| | | 1.35 eq. TMSCl | 33% (8.26 g) | |
| | | 4.4 eq. NMM | UPLC final | |
| | | 5 vol THF | solid: 98.0 | |
| | | 20 vol MTBE | area % | |
| | | 0° C.-rt, 30 min | | |
| 7 | 10.5 kg | 1.4 eq. 2-(aminooxy)ethanol | Isolated yield: | Ex. 13 |
| | | 1.5 eq. TMSCl | 54% (5.7 kg) | |
| | | 3.1 eq. NMM | HPLC final | |
| | | 6 vol THF | solid: 99.3 | |
| | | 10 vol THF | area % | |
| | | 0° C.-rt, 30 min | | |

a: Compound (II) added in 1 min.;
b: Slow addition of compound (II) (15 min);
c: Slow addition of compound (II) (32 min);
d: Compound (II) added over 1 h; 1^{st} isolation: THF/MTBE solvent exchange; Recrystallization # 1: THF/MTBE; and Acetic acid added to aid removal of the TMS group; and
e: 1^{st} isolation: THF/MTBE solvent exchange/some acetonitrile; Recrystallization # 1: THF/MTBE; Recrystallization # 2: EtOH/water (30:33 volume ratio); and Acetic acid added to aid removal of the TMS group.

Entries 3-4: The next two reactions used a slow addition of compound (II) slurry in MTBE. In both cases, compound (I) was 86-87 area % by HPLC, but there were a number of impurities. In particular, the cyclized impurity was high at 2.7-4.8% and a late eluting impurity at RRT 2.26 (1.7-2.3%). Under these conditions, no dimer impurity was observed in the in-process HPLC.

It was determined that a purification method was needed to remove both the dimer impurity at RRT 1.92 and the unknown non-polar impurity at RRT 2.26. It was found that partially dissolving the solid in four volumes of THF and precipitating the material with an anti-solvent proved effective at removing the dimer. The dimer impurity was cut in half when 16 volumes of IPAc was used as the anti-solvent. Furthermore, 86% of the dimer was removed when 16 volumes of toluene was used as the anti-solvent.

Entry 5: Acetic acid was used in the water/MTBE/THF isolation procedure to attempt to remove the TMS group. The pH of the aqueous was found to be pH 8.5 and it was hypothesized that this was due to the extra NMM added to the reaction with the pTSA salt (K-1). Therefore, a small amount of acetic acid was added to the aqueous layer to reduce it to pH 4.5. The isolation was continued and the initial precipitate was recrystallized from THF/MTBE. The purity of compound (I) was 99.4 area % with no single impurity greater than 0.14%. The product was isolated in a yield of 42%.

Entry 6: A 25 g pre-demonstration run was conducted using the new conditions in Entry 6. This reaction incorporated the acetic acid pH adjustment and new ethanol/water recrystallization conditions discussed in the next section. Compound (II) was added as a slurry in MTBE over 28 minutes, maintaining the reaction temperature below 4° C. The reaction was deemed complete after approximately 40 minutes. The batch was filtered to remove some solids and then was returned to a clean reactor. The batch was partially distilled under vacuum at which point solids precipitated. These solids were removed by filtration and were found to be 49 area % of the cyclized impurity. The filtrate was returned to the reactor and was treated with ten volumes of water, five volumes of ethanol, and one equivalent of acetic acid to promote TMS cleavage. The batch was stirred overnight, then was vacuum distilled with additions of water and acetonitrile and finally MTBE. The product was resistant to precipitate. The recovery was approximately 10 g once a solid was isolated and dried. The purity was not tested and the yield at this point was 40%. The product was dissolved in ethanol (30 vol) at 70° C. and 18 volumes of water was added maintaining 70° C., but no crystallization was observed. An additional 15 volumes of water was added to induce the cloud point. The batch was cooled and the solids collected and dried. The final yield was only 33% overall and the UPLC purity was 98.0 area %.

Preparation of Compound (I) (Entry 6): A reactor was charged with compound (K-1) (16.7 g, 0.067 mol, 1.25 eq.), THF (125 mL, 5 vol), and NMM (25.9 mL, 4.4 eq.). The pTSA salt dissolved in the mixture. TMSCl (9.2 mL, 1.35 eq.) was added at which point solids precipitated. Compound (II) (25.4 g, 0.054 mol, 1.0 eq.) was added as a slurry in MTBE (500 mL, 20 vol) over 28 min maintaining the reaction temperature below 4° C. The reaction was deemed complete after approximately 40 min. The batch was filtered to remove some solids and then was returned to a clean reactor. The batch was partially distilled under vacuum at which point solids precipitated. These solids containing the cyclized impurity were removed by filtration. The filtrate was returned to the reactor and was treated with 10 vol of water, 5 vol of ethanol, and 1 eq. of acetic acid to promote TMS cleavage. The batch was stirred overnight, and then was vacuum distilled with additions of water, acetonitrile, and finally MTBE. The product was resistant to precipitate. The recovery was approximately 10 g once a solid was isolated and dried. The purity was not tested and the yield at this point was 40%. The product was dissolved in ethanol (30 vol) at 70° C. and 18 vol of water was added maintaining 70° C., but no crystallization was observed. An additional 15 vol of water was added to induce the cloud point. The batch was cooled, and the solids collected and dried. The final yield was only 33% overall (8.26 g) and the UPLC purity was 98.0 area %.

In summary, several observations about the processes with the pTSA salt and MTBE based on those Entries of Table 11 are described below:
Using MTBE instead of THF eliminated the dimer impurity (RRT 1.92);
It was observed that when compound (II) was added slowly, the RRT 0.97 and RRT 2.26 impurities were reduced to 1.2% and 1.5%, respectively; and
The dimer impurity was effectively stopped from forming by keeping the batch temperature below 5° C. while charging compound (II) slurry in MTBE. The cyclized impurity (RRT 0.97) was removed by distilling the batch to 15 volumes, charging eight volumes of THF, stirring for one hour, and removing the solids by filtration.

### Recrystallization of the Compound of Formula (I)

For the recrystallization experiments, a batch of compound (I) in 97.2 area % was used to determine the effect of the recrystallization on the impurity profile. There were two recrystallization conditions explored (see Table 12). The first recrystallization involved a variation of conditions where the material was dissolved in hot ethanol, water was charged at the high temperature used to dissolve the material, and then the solution was slowly cooled. Entries 1 and 2 were run under these conditions. The final HPLC purities of Entries 1 and 2 were 97.64% and 97.50%, respectively. The cyclized impurity (RRT 0.97) dropped from 1.18% in the starting batch to 0.99% in Entry 1 and 1.11% in Entry 2, and the concentration of the dimer impurity (RRT 1.92) remained essentially unchanged. The second recrystallization involved dissolving the material in hot ethanol, slowly cooling the solution, and then charging water slowly to the solution at 15° C. Entry 3 was run under the second recrystallization condition. The final HPLC purity of Entry 3 was 97.50 area %, the cyclized impurity was 1.24 area %, and the dimer remained unchanged. The data suggested that adding the anti-solvent water at higher temperature followed by slow cooling removes cyclized impurity. This first recrystallization condition provided advantages in removing the cyclized impurity.

The recrystallization volumes were based on the exact mass of the crude product heading into the final recrystallization rather than the input of the reaction starting material compound (II). Therefore, it was important to dry the crude compound (I) prior to the recrystallization. When the conditions for recrystallization were applied to the 25-g pre-demonstration batch (Entry 4), additional volumes of water were required to reach the cloud point.

TABLE 12
Recrystallization of Compound (I)

| Entry | Conditions | Purity of Compound (I) |
|---|---|---|
| 1 | 25 vol EtOH, 85° C. | HPLC purity: 97.64 area % |
| | 15 vol Water at ≥70° C. | |
| | Isolation at 15° C. | |
| 2 | 17 vol EtOH,85° C. | HPLC purity: 97.50 area % |
| | 13 vol Water at ≥70° C. | |
| | Isolation at 15° C. | |
| 3 | 17 vol EtOH, 85° C. | HPLC purity: 97.50 area % |
| | 13 vol Water at 15° C. | |
| | Isolation at 15° C. | |
| 4 | 30 vol EtOH, 85° C. | UPLC purity: 98.0 area % |
| | 18 + 15 vol Water at ≥70° C. | |
| | Isolation at 15° C. | |

Further Development of the Amide Formation Via 2-(Aminooxy)Ethanol pTSA Salt (K-1)

Several reactions were conducted to optimize the reaction conditions and increase the purity of compound (I) as shown in Table 13.

TABLE 13
Optimizing the Amide Formation via 2-(aminooxy)ethanol pTSA salt (K-1)

| | | | | | | HPLC | |
|---|---|---|---|---|---|---|---|
| Entry | Scale | | | | Comp. (I) | purity | |
| No. | (g) | Conditions | | Conversion | (% yield) | (area %) | Isolation |
| 1 | 5 g | (K-1) | (1.25 eq.) | >99 | 58% | 97.6 | No Darco G60; and |
| | | TMSC1 | (1.35 eq.) | | (2.94 g) | | Isolated with 13.5 vol |
| | | NMM | (4.4 eq.) | | | | EtOH/10 vol water |
| | | THF | (5 vol) | | | | |
| | | MTBE | (20 vol) | | | | |
| 2 | 2 g | (K-1) | (1.25 eq.) | >99 | — | 82.6 | Not isolated |
| | | TMSC1 | (1.35 eq.) | | | | |
| | | TEA | (4.4 eq.) | | | | |
| | | THF | (5 vol) | | | | |
| | | MTBE | (20 vol) | | | | |
| 3 | 2 g | (K-1) | (1.25 eq.) | >99 | — | 59.1 | Not isolated |
| | | TMSC1 | (1.35 eq.) | | | | |
| | | DIPEA | (4.4 eq.) | | | | |
| | | THF | (5 vol) | | | | |
| | | MTBE | (20 vol) | | | | |
| 4 | 2 g | (K-1) | (1.25 eq.) | >99 | 47% | 95.1 | Darco G60 used; and |
| | | TMSC1 | (1.35 eq.) | | (0.95 g) | | Isolated with 13.5 vol |
| | | NMM | (4.4 eq.) | | | | EtOH/10 vol water |
| | | THF | (5 vol) | | | | |
| | | MTBE | (20 vol) | | | | |
| 5 | 10 g | (K-1) | (1.25 eq.) | >99 | 64% | 95.2 | No Darco G60; and |
| | | TMSC1 | (1.35 eq.) | | (6.4 g) | | Isolated with 13.5 vol |
| | | NMM | (4.4 eq.) | | | | EtOH/10 vol water |
| | | THF | (5 vol) | | | | |
| | | MTBE | (20 vol) | | | | |
| 6 | 20 g | (K-1) | (1.25 eq.) | >99 | 64% | 93.2 | No Darco G60; and |
| | | TMSC1 | (1.35 eq.) | | (12.96 g) | | Isolated with 13.5 vol |
| | | NMM | (4.4 eq.) | | | | EtOH/10 vol water |
| | | THF | (5 vol) | | | | |
| | | MTBE | (20 vol) | | | | |
| 7 | 50 g | (K-1) | (1.25 eq.) | >99 | 63% | 99.4 | Isolated with 13.5 vol |
| | | TMSC1 | (1.35 eq.) | | (31.6 g) | | EtOH/10 vol water; |
| | | NMM | (4.4 eq.) | | | | and |
| | | THF | (5 vol) | | | | treated with Darco |
| | | MTBE | (20 vol) | | | | G60 after initial |
| | | | | | | | isolation |
| 8 | 274 g | (K-1) | (1.37 eq.) | >99 | 44% | 98.7 | Isolated with 13.5 vol |
| | | TMSC1 | (1.35 eq.) | | (119.8 g) | | EtOH/10 vol water; |
| | | NMM | (4.4 eq.) | | | | and |
| | | THF | (5 vol) | | | | treated with Darco |
| | | MTBE | (20 vol) | | | | G60 after initial |
| | | | | | | | isolation |

Entry 1: The first reaction was a familiarization run on a 5 g scale using the latest conditions with pTSA salt (K-1) and MTBE as the co-solvent. This reaction was completed using the conditions previously developed; however, the isolation of compound (I) was different. Instead of a Darco G60 treatment and filtration, the reaction mixture was filtered to remove the NMM hydrochloride salt, then directly solvent exchanged to ethanol, and precipitated with water. After filtering and drying at 70° C. in a vacuum oven, light pink solids were obtained in 58% yield, 97.6 area % purity by HPLC analysis and 95.7 area % purity by UPLC analysis.

Entries 2-3: The next two reactions were investigated with a base other than NMM. One reaction was completed using triethylamine (TEA) as the base and the other reaction used DIPEA as the base. Both reactions went to completion, but the product was not isolated.

Entry 4: Another reaction was completed using the conditions as shown. After the reaction reached complete conversion, the reaction mixture was treated with Darco G60. This was then filtered and ten volumes of water was added. This was then distilled to 15 volumes and nine volumes of MTBE were added and distilled to 13 volumes (this was repeated twice). While isolating the product, a pink gumball formed and was brought back into solution using ten volumes of ethanol. This was then precipitated with water, and after filtering, the batch was dried in a 70° C. vacuum oven to obtain a 47% yield of light pink solids. The HPLC purity was 95.1 area %.

Entries 5-6: Two identical reactions were run on 10 g and 20 g scale. These were both isolated using no Darco G60 treatment and precipitated by water. In both cases the yield was 64%.

Entry 7: A 50 g reaction was run using the typical reaction conditions. The reaction went to complete conversion. After isolation of this batch using ethanol/water, the product was dissolved in ten volumes of THE at 40° C. and treated with Darco G60. The carbon was filtered off and the solution of product was returned to the reactor. After heating to 40° C., 20 volumes of MTBE was added and the batch was cooled. The product was isolated to give off-white solids after drying in a 70° C. vacuum oven. The purity was 99.4 area % by HPLC analysis and 99.4 area % by UPLC analysis. The purity was excellent for this batch. The high purity was primarily due to the use of the Darco G60 carbon treatment.

Entry 8: A demonstration run using MTBE and the pTSA salt (K-1) was run on 270 g scale. The reaction went to complete conversion. After isolation of this batch using ethanol/water, the product was dissolved in ten volumes of THE at 60° C., cooled to 40° C., and treated with Darco G60. The carbon was removed by filtration at 40° C. and the solution of product was returned to the reactor. After heating to 40° C., the batch was distilled to five volumes and ten volumes of MTBE was added over one hour. The batch was cooled over 13 hours. The product was isolated to give a 44% yield of off-white solids after drying in a 70° C. vacuum oven. The purity was 98.7 area % by HPLC analysis and 99.6 area % by UPLC analysis. The 44% overall yield for the process is an improvement over what was obtained in the pre-demo run (Entry 6 of Table 11).

Preparation of Compound (I) (Entry 8): A 20-L reactor was charged with compound (K-1) (201 g, 0.806 mol, 1.37 eq.), THF (1.5 L, 5.5 vol), and NMM (358 g, 3.54 mol, 4.4 eq. relative to (K-1)). The mixture was cooled to 0° C. TMSCl (118 g, 1.09 mol, 1.35 eq. relative to (K-1)) was added while maintaining the temperature at −5° C. In a 20-L carboy, compound (II) (274 g, 0.588 mol, 1.0 eq.) was added followed by MTBE (6 L, 21.8 vol). Compound (II) slurry was added over 1.25 h maintaining the reaction temperature below 5° C. The carboy was rinsed with MTBE (600 mL) and the rinse was added to batch. The batch was warmed to 20±5° C. and was stirred at that temperature for 30 min. In-process HPLC analysis indicated there was complete consumption of compound (II). The batch was filtered to remove some solids and the reactor and filter cake was rinsed with MTBE (2×600 mL). The filtrate was returned to a clean reactor (8 L total vol) and the batch was distilled under vacuum to a final volume of 1.35 L. Ethanol (2.7 L) was added to the reactor and the batch was distilled a second time to 1.35 L. Ethanol (2.7 L) was added and the batch was distilled a third time to approximately 1.5 L. The mixture was cooled to 20° C. and ethanol (2.3 L) was added. The mixture was warmed to 68° C., but the solids did not completely dissolve. At 70° C., water (2.7 L) was added over 2 h. All the solids dissolved with the addition of about 300 mL water. The mixture was cooled over 13 h to 10° C. The batch was aged at 10° C. for 4 h and filtered. The reactor and filter cake were rinsed with water (4×1.4 L). The wet cake (1131.3 g) was dried at 70° C. for four days to give 195.9 g of crude compound (I) (72%).

The crude compound (I) (195.9 g) and THF (2.7 L) were charged to a 10-L reactor. The batch was warmed to 54° C. to dissolve the product. Darco G60 (135 g, 50 wt %) was added and the temperature was adjusted to 40° C. The slurry was aged for 1.5 h and then was filtered to remove the carbon. The reactor and filter cake was rinsed with THF (2 L). The filtrate was returned to the cleaned reactor. The batch was vacuum distilled to 1.35 L and the batch temperature was adjusted to 40-41° C. MTBE (2.7 L) was fed to the mixture over 1 h maintaining the temperature at 40° C. The batch was cooled to 20° C. over 2 h and was aged at 20° C. for 1 h. The reactor and filter cake were rinsed with MTBE (2×540 mL). The wet cake weighed 246.6 g and was dried at 70° C. for two days to afford 119.8 g of compound (I) (44% yield). The ¹H NMR analysis of the product was consistent with the assigned structure and the UPLC purity was 99.6 area %.

### Example 8: Further Development of Amide Formation of Steps 6a) and 6b) via 2-(aminooxy)ethanol TsOH salt (K-1)

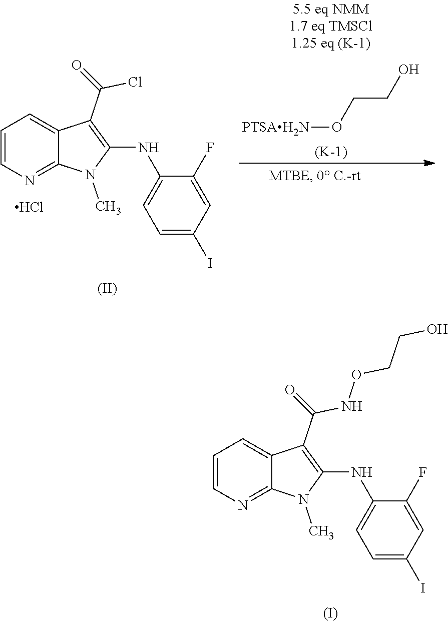

Comparison experiments were conducted using compound (K-1) or a free base of compound (K-1) in a single solvent of MTBE, as shown in Table 14.

TABLE 14
Amide Formation via 2-(aminooxy)ethanol pTSA salt (K-1)

| | | | | | HPLC | |
|---|---|---|---|---|---|---|
| Entry | Scale | | | Comp. (I) | purity | |
| No. | (g) | Conditions | Conversion % | (% yield) | (area %) | note |
| 1 | 20 g | (K-1) (1.25 eq.) | 99.8 | Batch-1: | 98.97 | Carbon treatment |
| | | TMSC1 (1.7 eq.) | | 49.8% | | performed after |
| | | NMM (5.5 eq.) | | (5.02 g) | | completion of the |
| | | MTBE (17 vol) | | | | reaction |
| | | 0° C.-rt, 30 min | | Batch-2: | 99.87 | Carbon treatment |
| | | | | 43.6% | | performed after |
| | | | | (4.39 g) | | isolation of the |
| | | | | | | crude |
| 2 | 10 | (K-1) (1.25 eq.) | 99.9 | 51.5% | 99.80 | Compound (K) from |
| | | TMSC1 (1.7 eq.) | | (5.2 g) | | (K-1) was used in |
| | | NMM (5.5 eq.) | | | | the coupling |
| | | MTBE (17 vol) | | | | |
| | | 0° C.-rt, 30 min | | | | |

Entry 1: the reaction was carried out with compound (K-1) directly in MTBE. Reaction conversion went as usual and the HPLC profile was comparable with Entry 2. After filtration and washes, combined filtrate (340 mL) was split into two equal portions. The crude product from one of the portions was isolated after carbon treatment and the recrystallized product was isolated from EtOH/H₂O with 49.8% yield in a purity of 98.97 area %. In another portion, crude product was isolated as usual from EtOH/H₂O and the crude was treated with Darco G-60 in THF just before crystallization. Isolated yield from this attempt was 45.6% and purity 99.87 area %.

Entry 2: Compound (K-1) was treated with 4.4 equivalent of NMM in MTBE. After 1 h, solids were removed by filtration, combined filtrate and wash were used for the amide coupling reaction. Solids analyzed by ¹H NMR and showed some losses of compound (K) during the initial base-treatment step of compound (K-1). Nevertheless, the amide coupling was performed on 10 g scale (Entry 2) using the solution of compound (K) and went for 99.88% conversion. The whole reaction was performed using 17 vol of MTBE (7 vol for forming a free base of compound (K-1) and 10 vol for compound (II) slurry) without charging THF. The crude compound (I) was isolated in a purity of 96.24 area %. This crude wet cake was transferred back into the reactor and dissolved in THF at 58.3° C. and the solution cooled down to 40-45° C. before charging Darco G-60. The batch agitated at 40° C. for 1 h with this charcoal and filtered off carbons and washed with THF. The solvent of the mixture was exchanged with EtOH.

Compound (I) was recrystallized from EtOH/H₂O with 51.5% yield and 99.8 area % by HPLC.

A. Recovery of Compound (K) from Base-Treatment of Compound (K-1)

In Entry 1 of Table 14, there was a stirring issue of the amide coupling reaction in 17 vol. of MTBE starting with compound (K-1). As shown in Entry 2 of Table 14, solids of NMM·TsOH salt during the initial base-treatment step were removed, and the main amide coupling reaction was expected to produce a less thick slurry. A series of experiments were conducted to understand and improve the recovery of compound (K) in the solution of MTBE/NMM from compound (K-1). The reactions performed by mixing NMM and compound (K-1) followed by MTBE were not reproducible as the solution could turn into a solid mass before charging MTBE. Table 15 shows experiments that successfully prepared compound (K) as a solution in MTBE/NMM.

TABLE 15
Isolation of Compound (K) as a Solution in MTBE/NMM

| Entry No. | 1 | 2 | 3 |
|---|---|---|---|
| Conditions | (K-1) (1.0 g, 1.25 eq.), | (K-1) (5.88 g, 1.1 eq), | (K-1) (26.74 g, 1.25 eq), |
| | MTBE (3 vol.), NMM (5.5 | MTBE (4 vol.), NMM | MTBE (4 vol.), NMM |
| | equiv.), rt, 15 min. 2 × 1.5 | (5.5 eq), rt, 15 min. 2 × | (5.5 eq), rt, 20 min. 2 × |
| | vol. MTBE washes | 1.5 vol. MTBE washes | 1.5 vol. MTBE washes |
| Weigh of the | 6.34 g | 52.8 g | 211.69 g |
| filtrate and | | | |
| washes | | | |
| qNMR potency of | 4.57 | 3.51 | 3.77 |
| compound (K) in | | | |
| solution | | | |
| % recovery of | 92.9 | 102% | 96.3 |
| Compound (K) in | | | |
| solution | | | |

Entry 1: NMM was charged into the slurry of compound (K-1) in MTBE and recovery of the amine in solution was 92.9%.

Entries 2 and 3: a slightly modified method was used to prepare an in-situ solution of compound (K) from compound (K-1) for use in the amide coupling reaction of steps 6a) and 6b).

### B. Amide Coupling Using In-Situ Prepared Solution of Compound (K)

The solution of compound (K) form Entry 2 of Table 15 was used in the amide formation. The amide coupling conversion was 99.15% with 84.15 area % overall purity of compound (I) in the reaction mixture. Crude compound (I) was isolated with a purity of 97.17 area %. Recrystallization of the crude material was performed in EtOH/H₂O. The HPLC purity of the isolated compound (I) was 99.47 area % in a yield of about 40%.

Several reactions were conducted using an in-situ prepared solution of compound (K) in the amide formation of steps 6a) and 6b), as shown in Table 16.

TABLE 16
Amide Formation Using an In-situ Prepared Solution of 2-(aminooxy)ethanol (K)

| Entry | Scale | | | | | |
|---|---|---|---|---|---|---|
| No. | (g) | | Conditions | Conversion | Crude Comp. (I) | Purified Comp. (I) |
| 1 | 40 | g | (K-1) (1.25 eq.) | 99.77% | Purity: 98.1 area % | Yield: 51% (20.45 g); |
| | | | TMSCl (1.7 eq.) | (94.1% purity) | | Purity: 99.93 area % |
| | | | NMM (5.5 eq.) | | | |
| | | | MTBE (7 + 11 vol) | | | |
| | | | 0° C.-rt, 30 min | | | |
| 2 | 20 | g | (K-1) (1.25 eq.) | 99.59% | Purity: 98.3 area % | Yield: 50% (10.1) |
| | | | TMSCl (1.7 eq.) | (91% purity) | | Purity: 99.89% |
| | | | NMM (5.5 eq.) | | | |
| | | | MTBE (7 + 11 vol) | | | |
| | | | 0° C.-rt, 30 min | | | |
| 3 | 340 | g | (K-1) (1.25 eq.) | 99.86% | Yield: 70% (240 g) | Yield: 55% (189 g) |
| | | | TMSCl (1.7 eq.) | (94.8% purity) | Purity: 98.8 area % | Purity: 99.88% |
| | | | NMM (5.5 eq.) | | | |
| | | | MTBE (7 + 11 vol) | | | |
| | | | 0° C.-rt, 30 min | | | |

Entry 1: The solution of compound (K) (Table 15, Entry 3) was used in the amide formation. Conversion of the amide coupling was 99.77% The crude product isolated from EtOH/H₂O was 98.1 area % of compound (I). Compound (I) was isolated by recrystallization from THF/MTBE in a 510% yield with a purity of 99.9 area %.

Entry 2: Similar conditions of Entry 1 was repeated. Compound (I) was isolated by recrystallization from THF/MTBE in a 50% yield with a purity of 99.9 area %.

Entry 3: The reaction was conducted on a scale of 340 g of compound (II). Compound (I) was isolated by recrystallization from THF/MTBE in a 55% yield with a purity of 99.9 area %.

Preparation of Compound (I) (Entry 3): To a 10-L reactor were charged compound (K-1) (227 g, 0.91 mol, 1.25 equiv.) and MTBE (1.36 L, 4.0 vol.) and start agitation at 20±5° C. Charged NMM (441 mL, 3.54 mol, 5.5 equiv.) and continued stirring the batch under same condition for 30 min. The batch was filtered after this time and the cake was washed with MTBE (2×0.51 L, 2×1.5 vol.). The combined filtrate and washes was transferred back into the reactor and cooled to 0° C. Slowly charged TMSCl (0.157 L, 1.24 mol, 1.7 equiv.) while maintaining the batch temperature below 5° C. The batch aged for 45 minutes before charging compound (II) slurry. In a 5 L 3-necked RBF equipped with mechanical stir were charged compound (II) (340 g, 0.73 mol, 1.0 equiv.) followed by MTBE (3.4 L, 10 vol.) and stirred for 35 min for uniform slurry before charge this slurry for amide coupling. The compound (II) slurry was transferred using a transfer pump over 1 h 20 min while maintaining the reaction temperature below 8° C. The RBF was rinsed with MTBE (0.34 L, 1 vol.) and added to the batch. The batch was continued stirring at 5° C. before warmed to 20±5° C. and stirred at that temperature for 30 min. After this time, in process control sample was pulled and HPLC analysis indicated 99.85% conversion of compound (II) to compound (I). The batch was filtered to remove all solids and the reactor was rinsed with THF (2×0.68 L, 2×2 vol.) and applied for cake wash. The filtrate was returned to a clean reactor and the batch was distilled under vacuum to a final volume of ˜1.7 L (5 vol.). Charged ethanol (3.4 L, 10 vol.) to the reactor and the batch was distilled a second time to ˜1.7 L (THF at 0.81 mol % to EtOH in ¹H NMR). The mixture was cooled to 20° C. and charged ethanol (2.89 L, 8.5 vol.) and water (0.68 L, 2 vol.). The mixture was warmed to 80° C. (all solids were not dissolved completely) and water (2.72 L, 8 vol.) was added over 2 h. The batch turned into a solution after charging ˜1.8 L of DI H₂O and remains a clear solution after completion of H₂O charge. The mixture was cooled over 13 h to 10° C. The batch was aged at 10° C. for 4 h before filtration. The reactor was rinsed with water (4×1.7 L) and transferred from reactor onto the cake. The wet cake (783 g) was dried at 60° C. for 3 days (Note: There was no weight loss after 26 h of drying) to give 240 g of crude compound (I) (70%). The HPLC purity of the crude compound (I) was 98.81 area % and KF (H₂O) was 0.28 wt %.

The crude compound (I) (238 g) and THF (3.4 L) were charged to a 10-L reactor. The batch was warmed to 51.2° C. (target was 60° C.) to dissolve the product. After complete dissolution, batch was cooled to 40° C. before charging Darco G60 (170 g, 50 wt %) and the slurry was aged for 30 min and then was filtered to remove the carbon (over 340 g celite). The reactor and filter cake was rinsed with THF (2×1.9 L, 2×3.5 vol.). The combined filtrate and washes was passed through a 0.2 m in-line filter and returned to the cleaned reactor. The batch was vacuum distilled to ˜1.7 L (5 vol.), and then heated to 60-65° C. for dissolution. Complete dissolution of the batch observed after charging additional THF (0.68 L, 1+1=2 vol.) then adjusted batch temperature to 40° C. and charged compound (I) Seeds (3.4 g). Continued stirring under same condition for 30 min before start dosing of MTBE (4.76 L, 14 vol.) to the mixture over 1 h 30 min while maintaining the batch temperature at 40° C. The batch was cooled to 20° C. over 2 h and was aged at 20° C. for one hour before filtration. The reactor and filter cake were rinsed with MTBE (2×0.68 L, 2×2 vol.). The wet cake weighed 455 g and was dried at 45° C. for 36 h to afford 189 g of compound (I) (55% yield). The ¹H NMR analysis of the product was consistent with the assigned structure and the HPLC purity was 99.88 area %.

### Example 9: Development of Chlorination of Step 5

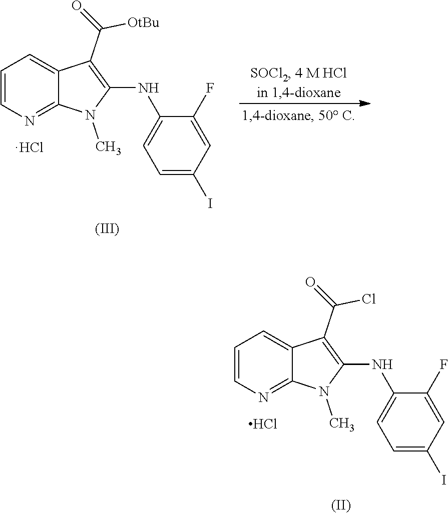

In some of processes as described herein, the workup and isolation of compound (II) involved multiple distillations with n-heptane to remove excess thionyl chloride. An improved process for the isolation was developed by dilution with n-heptane and filtration. Several Entries of the chlorination step to improve the isolation and the results are shown in Table 17.

TABLE 17
Chlorination of Step 5

| Entry | Scale | | Comp. (II) | HPLC Purity |
|---|---|---|---|---|
| No. | (g) | Conditions | (g) (% yield) | (area %) |
| 1 | 25.0 | 10.0 eq. thionyl chloride | 24.3 g | HPLC purity: |
| | | 6 eq. 4M HCl in | (97%) | 96.7 area % |
| | | 1,4-dioxane | | |
| | | 4.5 vol 1,4-dioxane | | |
| | | 55° C. for 24 h | | |
| 2 | 25.0 | 10.0 eq. thionyl chloride | 23.8 g | HPLC purity: |
| | | 6 eq. 4M HCl in | (95%) | 96.7 area % |
| | | 1,4-dioxane | | |
| | | 4.5 vol 1,4-dioxane | | |
| | | 55° C. for 24 h | | |
| 3 | 500.0 | 10.0 eq. thionyl chloride | 428 g | HPLC purity: |
| | | 6 eq. 4M HCl in | (91%) | 99.0 area % |
| | | 1,4-dioxane | | |
| | | 4.5 vol 1,4-dioxane | | |
| | | 55° C. for 24 h | | |

Entries 1 and 2: The reaction was diluted with n-heptane and filtered to generate clean compound (II) without the need for the repeated n-heptane distillations. The ¹H NMR analyses of compound (II) (Entry 1) was compared to a previous batch prepared by multiple distillations with n-heptane, and it showed that the process by dilution with n-heptane provided relatively more pure product of compound (II). The product (24.3 g, 97.4%) was isolated as a light grey solid. Entry 2 repeated the results of Entry 1. Entries 1 and 2 were analyzed immediately after the material was dried and demonstrated that the improvement made by this purification technique.

Entry 3: The demonstration batch of the acid chloride formation is shown. Accordingly, the isolation was accomplished by simply diluting the reaction with n-heptane and filtering the resulting solid. The product (428 g, 86% yield) was isolated as a light grey solid with an HPLC purity of 99.0 area %.

### Preparation of Compound (II) (Entry 3)

To a 10-L, jacketed reactor inerted under nitrogen was vented to a carboy containing an aqueous sodium hydroxide scrubbing solution. The reactor was charged with compound (III) (500 g, 1.07 mol) and 1,4-dioxane (2.25 L, 4.5 vol). The batch was stirred and was adjusted to 19° C. Thionyl chloride (0.776 L, 10 eq.) was added over 10 min and the temperature increased to 26° C. To the batch was added 4 M HCl in dioxane (1.6 L, 6.4 mol, 6 eq.) over 15 min at a batch temperature of 25° C. The batch was heated to 50° C. and was aged for 24 h. In-process analysis indicated the reaction was complete. The batch was cooled to 22.5° C. n-Heptane (3.25 L, 6.5 vol) was added to the batch and the batch was stirred for 30 min. The slurry was filtered and the filter cake was rinsed with n-heptane (1.5 L, 3 vol). The batch was conditioned on the filter overnight under nitrogen and was dried at 25-35° C. in a vacuum oven. The isolated yield was 91% (428 g). The ¹H NMR analysis of the product was consistent with the assigned structure. HPLC analysis: 99.0 area %.

The advantages to using this new isolation procedure include:
Less chance of decomposition. The purity of compound (II) was most likely to decrease during the heated distillation steps through the intramolecular cyclization pathway.
Much shorter batch times since the distillations would typically add two days to the process.
Performing the distillations with heptane ultimately dilutes the thionyl chloride, which is undesirable because this needs to be quenched before it can be disposed as waste. Without diluting the thionyl chloride, it can be quickly and efficiently quenched in a single reactor, without the need to quench multiple drums of distillates.

To protect the equipment for drying compound (II) over a long period of time at an elevated temperature, the isolation procedure was further optimized. Accordingly, the filtered wet cake was washed with 4×3.3 vol. of n-heptane to get almost neutral (pH˜6-7) filtrate.

To reduce dioxane as a residual solvent in compound (II), the drying of compound (II) can be conducted at an elevated temperature (35-38° C. or about 40° C.).

### Example 10: Development of Chlorination and Aniline Formations of Steps 4a) and 4b)

### A. Chlorination of Step 4a)

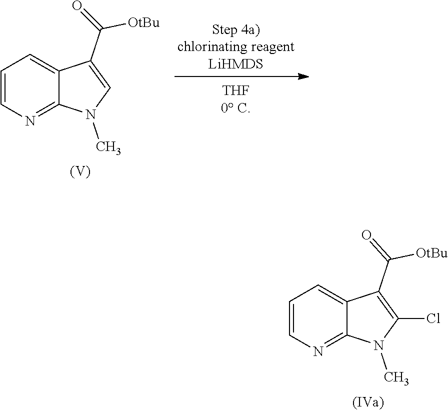

The reaction was performed by adding LiHMDS to a solution of the indole (IV) and chlorinating reagent in THE at 0° C. The reaction using hexachloroethane went to completion using only 1.1 eq. of the chlorinating reagent and 1.05 eq. of base. The reaction with 1.1 eq. of tosyl chloride went 95% completion. See Table 18. It was demonstrated that residual tosyl byproducts were completely removed with 1 M NaOH base washes. An increased amount of base and tosyl chloride would be expected to drive the reaction to completion. Since it has been demonstrated the tosyl byproducts can be removed by extraction, additional equivalents of tosyl chloride will not affect the purity of the final product (IVa).

TABLE 18
Chlorination of Step 4a)

| Entry | Scale | | Comp. (II) |
|---|---|---|---|
| No. | (g) | Conditions | (g) (% yield) |
| 1 | 0.203 | 1.0 eq. compound (V) | 0.212 |
| | | 1.1 eq. hexachloroethane | (91%) |
| | | 1.05 eq LiHMDS | |
| | | 10 vol THF | |
| | | 0° C., 15 min. | |
| | 0.199 | 1.0 eq. compound (V) | 0.183 |
| | | 1.1 eq. tosyl chloride | (80%) |
| | | 1.05 eq. LiHMDS | |
| | | 10 vol THF | |
| | | 0° C., 15 min. | |

### B. Aniline Formation of Step 4b)

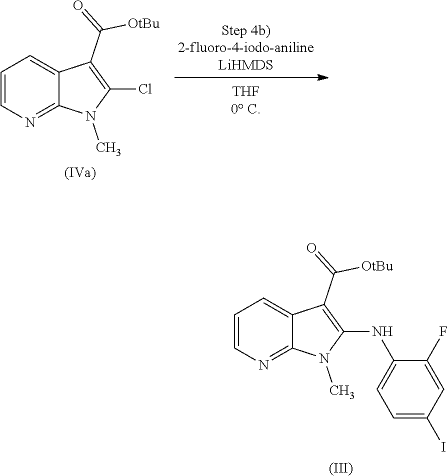 [SMILES: Cn1c(Cl)c(C(=O)OC(C)(C)C)c2cccnc21 ; Cn1c(Nc2ccc(I)cc2F)c(C(=O)OC(C)(C)C)c2cccnc21 ; "(III)"]

The reaction was performed by adding a solution of the 2-chloro-azaindole (IVa) to a solution of LiHMDS and 2-fluoro-2-iodoaniline (abbreviated as aniline) in THF at 0° C. (see Entry 1 of Table 19). The reaction was dose controlled, and after the internal temperature returned to the starting temperature, the reaction was complete. As two equivalents of base appear to be required due to the N—H of compound (III) being more acidic than the aniline N—H, a slight excess of this amount was used at the beginning of the reaction. The reaction was fairly clean, with the excess aniline remained in the sample after work-up.

The above reaction was performed on a 31 g scale (see Entry 2 of Table 19). Since the amount of aniline in the pot was kept to 0.98 equivalents, there was no accumulation of aniline in the reaction mixture upon complete consumption of starting material. The product was slurried in MTBE and filtered, and then the filtrate was further concentrated and reslurried in MTBE to obtain a second crop of material with a purity of >95%.

TABLE 19
Aniline Formation of Step 4b)

| Entry | Scale | | Comp. (II) | |
|---|---|---|---|---|
| No. | (g) | Conditions | (g) (% yield) | Purity |
| 1 | 0.212 | 1.0 eq. compound (IVa) | 0.412 g | — |
| | | 1.1 eq. aniline | (>100%) | |
| | | 2.3 eq. LiHMDS | | |
| | | 13 vol THF | | |
| | | 0° C., 15 min. | | |
| 2 | 31 g | 1.0 eq. compound (IVa) | 49.7 g | Purity >95% |
| | | 0.98 eq. aniline | (89%) | |
| | | 2.3 eq. LiHMDS, | | |
| | | 4.8 eq. THF, 0° C., 1 h. | | |

### C. One-Pot Reaction of Steps 4a) and 4b)

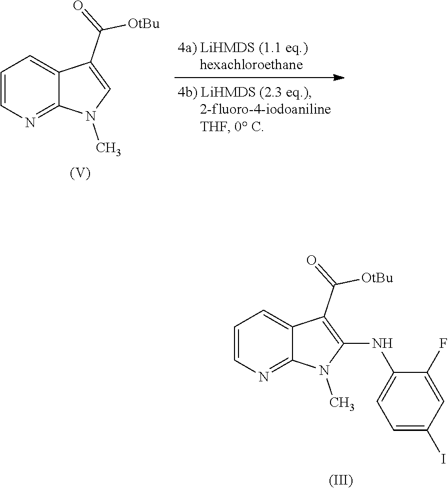

The reaction was performed by charging the flask with the 7-azaindole-3-t-butyl ester and hexachloroethane, dissolving the materials in THF and cooling to 0° C., and adding 1.1 eq. LiHMDS while maintaining the temperature below 4.2° C. The chlorinated compound (IVa) from step 4a) had a 97.1% purity as indicated by HPLC. The flask was charged with aniline at 0° C., and 2.3 eq. LiHMDS were added dropwise while maintaining the temperature below 6.1° C. The aniline was used as the limiting reagent, and a second portion of aniline was added to reduce the remaining chlorinated compound (IVa) from 8.7% to 3.9%. After aqueous work-up, the sample was slurried in 2 vol of MTBE, filtered, and dried to give product (18.66 g, 83%) as a light brown solid (see Entry 1 of Table 20).

The above reaction was performed on a 20 g scale (see Entry 2 of Table 20). The reaction proceeded smoothly and product (38.9 g, 97%) was isolated as an orange solid with a purity of 92% as indicated by HPLC. All impurities can be removed by work-up as noted above.

TABLE 20
One-pot Reaction of Steps 4a) and 4b)

| | | | Comp. (II) |
|---|---|---|---|
| Entry No. | Scale (g) | Conditions | (g) (% yield) |
| 1 | 11.2 | 1.0 eq. compound (V) | 18.66 g |
| | | 1.1 eq. hexachloroethane | (83%) |
| | | 0.96 eq. aniline | |
| | | 3.4 eq LiHMDS | |
| | | 20 vol THF, 0° C., 7 h. | |
| 2 | 20.0 | 1.0 eq. compound (V) | 38.87 g |
| | | 1.1 eq. hexachloroethane | (97%) |
| | | 1.0 eq. aniline | |
| | | 3.4 eq LiHMDS | |
| | | 20 vol THF, 0° C., 6 h. | |

### Example 11: Further Development of Chlorination and Aniline Formations of Steps 4a) and 4b)

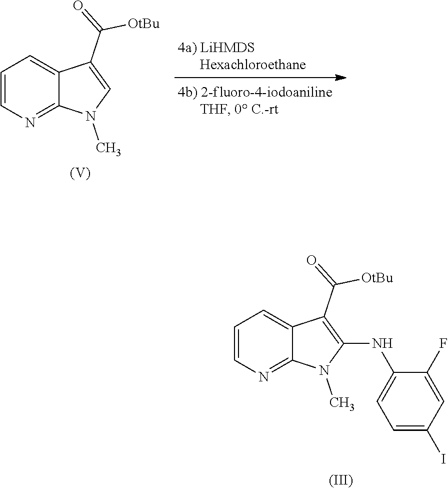

### A. Initial Optimization

For the development work for the processes to compound (II) and compound (I), several batches of compound (III) was prepared as shown in Table 21.

TABLE 21
Chlorination and Aniline Formations of Steps 4a) and 4b)

| Entry | Scale | | Comp. (III) | Isolation/ |
|---|---|---|---|---|
| No. | (g) | Conditions | (g) (% yield) | Purity |
| 1 | 15.0 | 1.0 eq. compound (V) | 26.1 g | New isolation |
| | | 1.05 eq. hexachloroethane | (86%) | procedure: |
| | | 1.1 eq. aniline | | Solvent |
| | | 3.5 eq. 1M LiHMDS | | exchange |
| | | 7 vol THF | | to ethanol. |
| | | 0° C. (30 min); rt for 16 h | | |
| 2 | 15.0 | 1.0 eq. compound (V) | 28.2 g | — |
| | | 1.05 eq. hexachloroethane | (93%) | |
| | | 1.1 eq. aniline | | |
| | | 3.5 eq. 1M LiHMDS | | |
| | | 7 vol THF | | |
| | | 0° C. (30 min); rt for 16 h | | |
| 3 | 15.0 | 1.0 eq. compound (V) | 26.9 g | — |
| | | 1.05 eq. hexachloroethane | (89%) | |
| | | 1.1 eq. aniline | | |
| | | 3.5 eq. 1M LiHMDS | | |
| | | 7 vol THF | | |
| | | 0° C. (30 min); rt for 16 h | | |
| 4 | 280.0 | 1.0 eq. compound (V) | 543 g | HPLC purity: |
| | | 1.1 eq. hexachloroethane | (96%) | 97.9 area % |
| | | 1.1 eq. aniline | | ¹H NMR wt % |
| | | 3.5 eq. 1M LiHMDS | | assay: |
| | | 7 vol THF | | 98.0 wt % |
| | | 0° C. (30 min); rt for 16 h | | |

As utilized in the workup of step 6b) reaction, if the THF/water mixture was solvent exchanged to ethanol/water, the material did not shell to the side of the reactor. This was applied to the demonstration batch of compound (III).

The aniline installation was the first step in the three-step process to complete compound (I) demonstration batch (280 g scale, Entry 4 of Table 21). This reaction was also performed to demonstrate the new isolation strategy, which after ammonium chloride quench, the solvent was exchanged from THF to ethanol instead of THF to water. The new isolation technique prevented the “shelling” issue without impacting either the yield or the purity. The product (543 g, 96% yield) was isolated as a beige solid with a purity of 97.9% by HPLC.

### Preparation of Compound (III) (Entry 4)

To a 10-L, jacketed reactor inerted under nitrogen was charged lithium bis(trimethylsilyl)amide (1.0 M, 4.2 L, 4.2 moles, 3.5 eq.). The mixture was cooled to −3.5° C. A carboy was charged with compound (V) (280 g, 1.2 mol, 1.0 eq.), hexachloroethane (317 g, 1.33 mol, 1.1 eq.), and THF (1.24 L, 4.4 vol). The mixture in the carboy was stirred to make a homogeneous solution. The THF solution containing compound (V) and hexachloroethane was charged to the reactor over 26 min. The batch temperature increased to 7° C. during the feed. The batch was agitated for 1 h at 5° C. In-process HPLC analysis showed that the conversion was greater than 98%. A clean carboy was charged with 2-fluoro-4-iodoaniline (314 g, 1.33 mol, 1.1 eq.) and THF (498 mL, 1.8 vol). The mixture was stirred to dissolve the solids and then the solution was transferred to the reactor over 33 min. The temperature during the addition reached 6.9° C. The batch temperature was adjusted to 15° C. and aged for 14 h. In-process HPLC analysis showed greater than 99% conversion to compound (III). The batch was cooled to 2° C. The reaction was quenched by the addition of saturated ammonium chloride (1.1 L, 3.9 vol) over 25 min. The batch was vacuum distilled (starting volume 7.7 L) to a final volume of 2.2 L. Water (1.4 L, 5 vol) was charged with the batch at 45-50° C. Ethanol (2.5 L, 8.9 vol) was charged at a batch temperature of 30-40° C. The batch was vacuum distilled (starting volume 6.5 L) to a final volume of 4.2 L. Ethanol (1.4 L, 5 vol) was charged at a batch temperature of 54° C. The batch was cooled to 20° C. and was stirred 9 h. The slurry was filtered and was rinsed with ethanol (2×0.84 L, 3 vol) and water (2.8 L, 10 vol). After drying the batch at 40-50° C., compound (III) was obtained in 96% yield (543 g). The ¹H NMR analysis was consistent with the assigned structure. Karl-Fischer analysis: 0.07% water. NMR weight assay: 98.0 wt %. HPLC analysis: 97.9 area %.

### B. Further Optimization

While the developed process was robust with respect to product purity and reaction yield, the reaction may not be volume efficient (see Entry 1 of Table 22). To further optimize the process, several batches of compound (III) was prepared according to the reaction conditions as shown in Table 22.

TABLE 22
Chlorination and Aniline Formations of Steps 4a) and 4b)

| Entry | Scale | | Comp. (III) | |
|---|---|---|---|---|
| No. | (g) | Conditions/(% conversion) | (g) (% yield) | Isolation/Purity |
| 1 | 350 | 1.0 eq. compound (V) | 646 g | HPLC purity: 99.67 area % |
| | | 1.1 eq. hexachloroethane | (91.8%) | Total reaction vol: 23 vol |
| | | 1.05 eq. aniline | | Isolation: see Entry 4 of |
| | | 3.5 eq. 1M LiHMDS | | Table 21 |
| | | 7 vol THF | | |
| | | 0-5° C. (30 min); rt for 14.5 h | | |
| 2 | 3.5 g | 1.0 eq. compound (V) | 6.5 g | HPLC purity: 99.01 area % |
| | | 1.1 eq. hexachloroethane | (93.0%) | Total reaction vol: 23 vol |
| | | 1.05 eq. aniline | | Isolation: modified work- |
| | | 3.5 eq. 1M LiHMDS | | up procedure |
| | | 7 vol THF | | |
| | | 0-5° C. to rt | | |
| | | Step 4a): 1 h (>99.5%) | | |
| | | Step 4b): 18 h (94.5%) | | |
| 3 | 3.5 g | 1.0 eq. compound (V) | 4.6 g | HPLC purity: 99.2 area % |
| | | 1.1 eq. hexachloroethane | (65.0%) | Total reaction vol: 9.5 vol |
| | | 1.05 eq. aniline | | Isolation: by adding IPA/ |
| | | 1.2 eq. 1.5M LiHMDS (step 4a) | | water (10%, 18 vol) |
| | | 3.0 eq. t-BuOK (step 4b) | | |
| | | 6 vol THF | | |
| | | 0-5° C. to rt | | |
| | | Step 4a): 0.5 h (99.5%) | | |
| | | Step 4b): 20 h (95%) | | |

Modified work up procedure:
1) Reaction mixture quenched with NH₄Cl (aq) (saturated, 4 vol);
2) IPA/water (¼, 300 vol) was added to reaction mixture at rt and stirred for overnight;
3) Mixture was cold down to 0° C. and filtered and washed with IPA/water (¼, 30 vol);
4) Filter cake was slurred in IPA (5 vol) at rt for 1 h; and
5) Mixture was filtered, washed with IPA (2 vol) and dried in vacuum oven at 35° C. to obtain a light brown solid.

Entry 3: Combination of LiHMDS (for step 4a), to form intermediate chlorate derivative) and t-BuOK (for step 4b), to form the product) were used as the base and reaction went to completion. Accordingly, LiHMDS in 1.5 M solution was reduced to 1.2 equiv and the final reaction volume was reduced to 9.5 vol, by using solid t-BuOK. While the optimized reaction achieved the same conversions as the previous developed process, isolation by further reducing volumes of IPA/water remained optimization.

### Example 12: Development of Iodination and Aniline Formations of Steps 4a) and 4b)

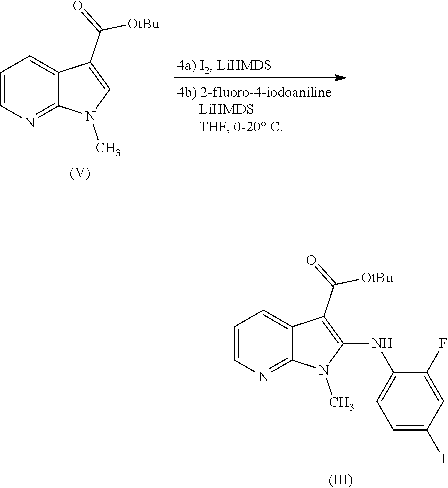

Using I₂, the only byproduct formed is LiI. The reaction was set up the same as with hexachloroethane according to Example 8A, where compound (V) and iodine were first charged to the flask, and LiHMDS (1.1 eq.) was added dropwise to a solution of this mixture at 0° C. Upon addition of the last drops of LiHMDS, the deep, dark iodine color disappeared and the solution turned a clear, light orange color. However, HPLC analysis revealed mostly starting material. Upon addition of an additional 0.1 eq. of LiHMDS and warming the reaction to room temperature, the reaction (step 4a)) proceeded nearly to completion. The reaction was continued analogously to the established procedure (e.g., Example 8B), with the only change being that the reaction was allowed to slowly warm to rt after complete addition of the LiHMDS. The S_{N}Ar reaction appeared to progress smoothly. After the MTBE slurry, the product was isolated (0.82 g, 82%) as an off-white solid with 96.3% HPLC purity (see Entry 1 of Table 23).

TABLE 23
Iodination and Aniline Formations of Steps 4a) and 4b)

| | | | Comp. (III) |
|---|---|---|---|
| Entry No. | Scale (g) | Conditions | (g) (% yield) |
| 1 | 0.50 | 1.0 eq. compound (V) | 0.82 g |
| | | 1.05 eq. 12 | (82% yield) |
| | | 3.4 eq. LiHMDS | |
| | | 0.96 eq. 2-fluro-4-iodoaniline | |
| | | 4 vol THF, 0° C.-rt | |

### A. Iodination of Step 4a)

The iodination appears to go through a different mechanism than the chlorination reaction. The loss of the iodine color after complete addition of the LiHMDS coupled with the reaction containing mostly starting material implies the formation of an in situ generated iodination species. The reaction was performed to verify the necessity of >1 eq. of LiHMDS for the iodination reaction to occur. Indeed, when only 0.85 eq. of LiHMDS were used in the reaction, only starting material was observed by HPLC. A tentative mechanism for formation of N-Iodo HMDS in situ is shown below:

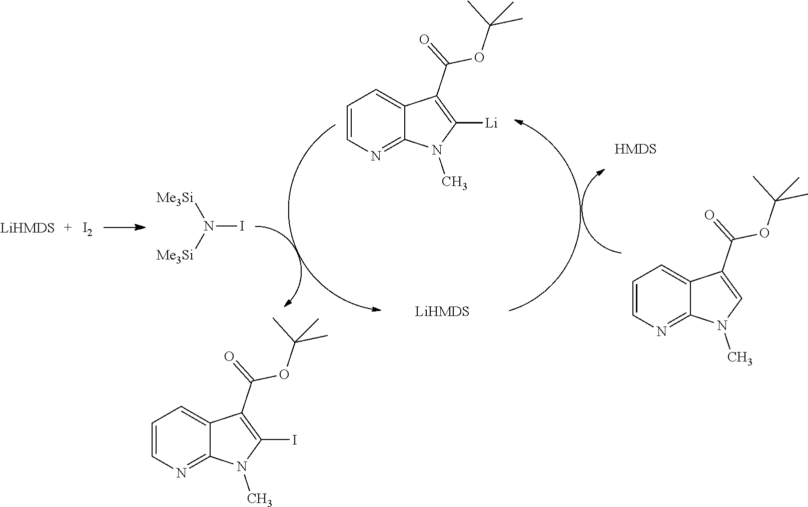

The N-iodoHMDS is sensitive to hydrolysis. As LiHMDS has been added to a solution of the indole (V) and iodine, LiHMDS reacts with iodine first, by the time an excess of LiHMDS is added, the iodination species has already begun to decompose. Upon adding an additional 0.5 eq. of I₂ as a solution in THF, the iodination reaction went to completion. Subsequently, the S_{N}Ar reaction proceeded as usual.

In view of the above, the order of additions of compound (V), iodine, and LiHMDS was altered, where a solution of compound (V) and iodine were added to a solution of LiHMDS. This way, the in situ iodinating reagent should react with the indole before any type of degradation can occur.

### B. One-Pot Reaction of Steps 4a) and 4b) Via Iodination

The order of the addition was rearranged to add a solution of compound (V) and I₂ in 5 volumes of THF to a solution of LiHMDS. HPLC indicated the completion of the iodination reaction. A solution of aniline in 2 volumes of THF is added dropwise to the 2-iodo azaindole (IVb) solution. HPLC indicated the completion of the S_{N}Ar reaction. The reaction was quenched with sat. NH₄Cl and solvent swapped to water to give the crude material. Suspending the crude material in MTBE and solvent swapping to EtOH gave product (12.4 g, 69%) as a light tan solid (see Entry 1 of Table 24). It is worth noting that the yield for this reaction was 72% on a 100 g scale.

The above reaction was performed on a 132 g scale. The desired product was precipitated from water by distilling off the THF after quenching the reaction. The material was slurried in ethanol. Compound (III) has very low solubility in ethanol, and compound (V) and the 2-fluoro-4-iodoaniline are moderately soluble in ethanol. The product of compound (III) (232 g, 86%) was isolated as an off-white solid.

TABLE 24
One-port Reaction of Steps 4a) and 4b) via Iodination

| | | | Comp. (III) |
|---|---|---|---|
| Entry No. | Scale (g) | Conditions | (g) (% yield) |
| 1 | 9.0 | 1.0 eq. compound (V) | 12.4 g |
| | | 1.2 eq. 12 | |
| | | 3.5 eq. LiHMDS | (69%) |
| | | 0.96 eq. 2-fluro-4-iodoaniline | |
| | | 4 vol THF, 0° C.-rt | |
| 2 | 132 | 1.0 eq. compound (V) | 228 |
| | | 1.1 eq. I2 | (86%) |
| | | 3.5 eq. LiHMDS | |
| | | 1.0 eq. 2-fluro-4-iodoaniline | |
| | | 7 vol THF, 0° C.-rt | |

### Example 13: Process for Preparing 2-((2-fluoro-4-iodophenyl)amino)-N-(2-hydroxyethyl)-1-methyl-1H-pyrrolo[2,3-b]pyridine-3-carboxamide (i.e., Formula (I))

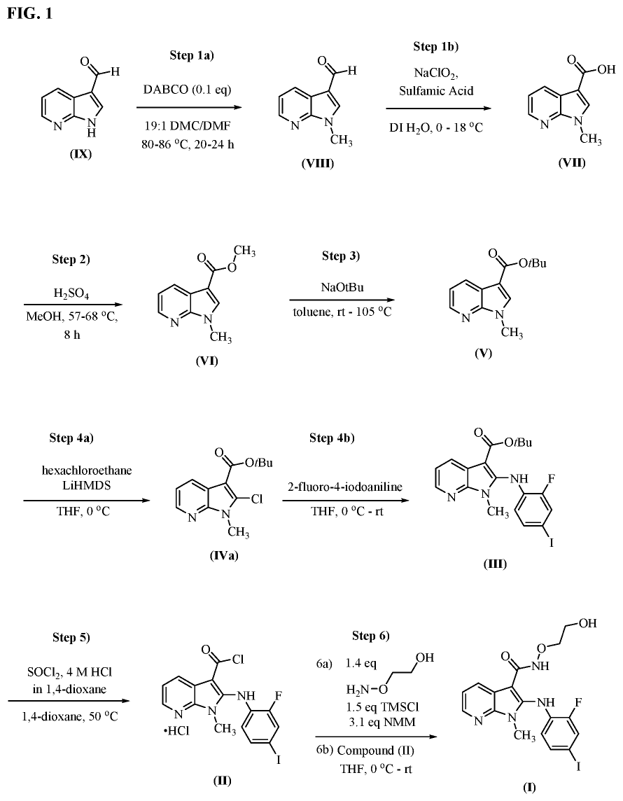

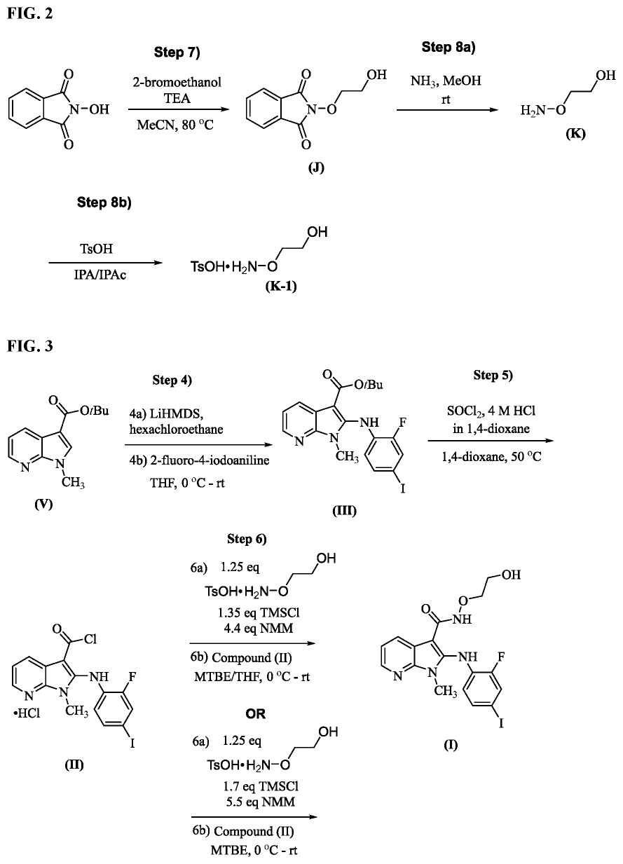

The compound of formula (I) was prepared according to Steps as shown in FIG. 1.

### Steps 1a) and 1b): Preparation of the Compound of Formula (VII)

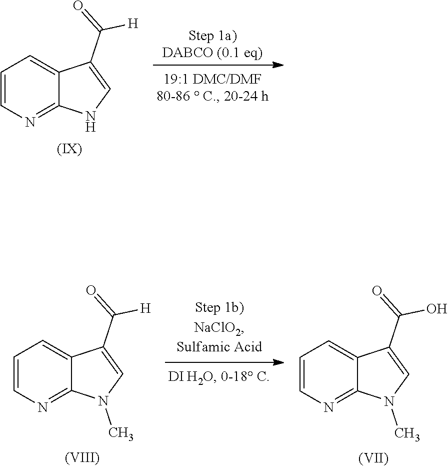

To a 400 L reactor was charged compound (IX) (17.0 kg), DABCO (1.31 kg), and dimethyl carbonate (164 kg, 9 vol). Stirring was started, dimethylformamide (16.0 kg, 1 vol) was charged, and the reactor was heated to 87.4° C. for 24 h. HPLC analysis showed 99.58% conversion, so the batch was cooled to 20-30° C. and was vacuum distilled (27.5 in Hg, 35.1° C.) to a final volume of 87 L (5 vol). Ethyl acetate (153 kg, 10 vol) was charged to the reactor and the batch was vacuum distilled (27.5 in Hg, <40° C.) to a final volume of 84 L (5 vol). EtOAc (153 kg, 10 vol) was charged to the reactor, and the batch was vacuum distilled (27.5 in Hg, <40° C.) to a final volume of 85 L (5 vol), and then the temperature was adjusted to 15-25° C.

To a separate vessel, a citric acid solution was prepared by charging DI H₂O (50 L, 3 vol), citric acid (6.70 kg), and was stirred for 45 minutes to fully dissolve the solids. The citric acid solution was added to the reactor over 1 hour with stirring. (Note: the citric acid solution addition is slightly exothermic). EtOAc (46 kg, 3 vol) was charged to the reactor and the batch was stirred for 30 min at 15-25° C. The layers were separated (which took 20 minutes), the aqueous layer was recharged to the reactor, followed by EtOAc (123 kg, 8 vol). The layers were stirred for 20 min, were separated, the aqueous layer was recharged, followed by EtOAc (123 kg, 8 vol). The layers were stirred for 20 min, were separated, and the combined EtOAc layers were recharged to the 400 L reactor. The batch was vacuum distilled (27.5 in Hg, <40° C.) to a final volume of 80 L (5 vol). ¹H NMR revealed 0% residual DABCO remained, so DI H₂O (171 L, 10 vol) was charged over 30 min while maintaining the internal temperature <55° C. (Note: the water addition is exothermic). The batch was vacuum distilled (29.1 in Hg, <55° C.) to a final volume of 84 L (5 vol), and the batch was adjusted to the 13.6° C.

To a separate vessel, a sulfamic acid solution was prepared by charging DI H₂O (170 L, 10 vol), sulfamic acid (28.2 kg), and stirring for 20 min. (Note: all solids may not dissolve). The sulfamic acid solution was added to the reactor with stirring over a 15 min period while maintaining the internal temperature at 8-18° C. A sodium bisulfite scrubber (48.0 kg; 250 L DI H₂O) and attached to the reactor. To a separate vessel, a sodium chlorite solution was prepared by charging DI H₂O (85.0 L, 5 vol), sodium chlorite (25.0 kg), and was stirred for 30 min. The sodium chlorite solution was charged to the reactor over a 6 h period, with an N₂ flow rate of 60 L/min, while maintaining the internal batch temperature between 8-18° C. The batch temperature was then adjusted to 6.7° C. and the batch was transferred to the Rosenmund hastelloy agitated filtered and was conditioned until liquids stopped eluting. The reactor was charged with DI H₂O (37.0 L, 2 vol) and the rinse was passed over the solids and was conditioned until liquids stopped eluting. The reactor was again charged with DI H₂O (36.0 L, 2 vol) and the rinse was passed over the solids and was conditioned until liquids stopped eluting. The solids were transferred to a vacuum oven and dried at 45-55° C. for 100 h to give product (VII) (12.7 kg, 62%).

Specifications of obtained solid: ¹H NMR (consistent with the compound (VII)); appearance: light yellow solid; KF (% water): 0.60%, ¹H NMR (d₆-DMSO) weight assay versus 1,4-dimethoxybenzene (92.29%); and HPLC purity (area % @ 247 nm): 77.8%.

### Step 2): Preparation of the Compound of Formula (VI)

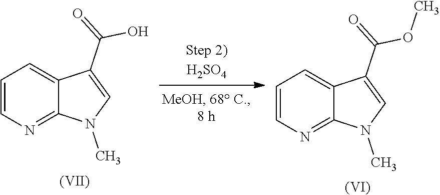

To a 400 L reactor, inerted with N₂ flow at 10 L/min for 19 h, was charged compound (VII) (12.7 kg) and Methanol (202 kg, 20 vol). The stirring, which was performed at 60 RPM, was started and the batch temperature was adjusted to 10° C. Concentrated sulfuric acid (23.4 kg, 1 vol) was charged over 45 min period while maintaining the batch temperature at 10-20° C. (Note: this addition is exothermic.) The batch temperature was adjusted to 58-68° C. and was maintained in this range for 21 h. The batch was cooled to 15-25° C., and HPLC analysis revealed that compound (VI) was formed >97% relative to compound (VII), so the batch was vacuum distilled (28 in Hg, <40° C.) to a final volume of 64 L (5 vol).

In a separate vessel, a sodium hydroxide solution was prepared by mixing DI H₂O (154 L, 12 vol) with 50 wt % sodium hydroxide (18.3 kg) with stirring. (Note: this addition is exothermic). The batch was cooled to 9.8° C., and the sodium hydroxide solution was added to the reactor over 45 min while maintaining the batch temperature at 10-20° C. (Note: this addition is exothermic). Upon completion of the addition, the pH was 1.73. In a separate vessel, a sodium bicarbonate solution was prepared by charging DI H₂O (38 L, 3 vol), sodium bicarbonate (3.67 kg), and stirring for 30 min until all solids were fully dissolved. The sodium bicarbonate solution was charged to the reactor over 20 min, and upon completion of the addition, the pH was 6.66. The batch temperature was adjusted to 15-25° C. and the batch was transferred to the Rosenmund hastelloy agitated filtered and was conditioned until liquids stopped eluting. The reactor was charged with DI H₂O (101 L, 8 vol), the rinse was transferred from the kettle onto the cake as a displacement wash. The reactor was charged with DI H₂O (38.1 L, 3 vol), the rinse was transferred from the kettle to the solids, and was conditioned until liquid stopped eluting. The product was dried under nitrogen flow at 50° C. for 10 days to give compound (VI) (12.1 kg, 88% yield).

Specifications of obtained solid: ¹H NMR (consistent with compound (VI)); appearance: off-white solid; KF (% water): 0.52%; ¹H NMR (d₆-DMSO) weight assay versus 1,4-dimethoxybenzene (90.84%); and HPLC purity (area % @ 247 nm): 97.2%.

### Step 3): Preparation of the Compound of Formula (V)

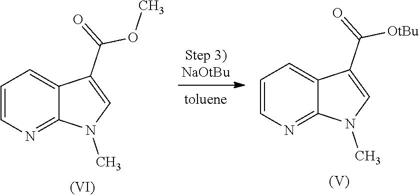

To a 400 L reactor, inerted with N₂ flow at 20 L/min for 20 h, was charged compound (VI) (12.1 kg), sodium tert-butoxide (21.4 kg), and anhydrous toluene (109 L, 9 vol) which was treated with StatSafe (50 ppm). The batch was stirred 80 RPM, was heated over the course of 90 min to 103° C., was held at this temperature for 45 min, and was cooled to −5-5° C. HPLC analysis showed 94.3% product.

In a separate vessel, a sodium bicarbonate solution was prepared by charging DI H₂O (54.5 L, 4.5 vol), ammonium chloride (20.1 kg), and stirring the solution until all solids are fully dissolved. A 2 M HCl scrubber was attached to the reactor, and the ammonium chloride solution was charged to the reactor over 5 h while maintaining the batch temperature at 5-10° C. (Note: this addition is extremely exothermic, and heating above 15° C. will lead to decomposition). DI H₂O (73.0 L, 6 vol) was charged to the reactor and the batch temperature was adjusted to 15-25° C. (Note: The DI H₂O addition is slightly exothermic). EtOAc (44 kg, 4 vol) was charged, the batch was stirred for 15 minutes, and the layers were separated. The aqueous layer was recharged to the reactor, followed by EtOAc (87.5 L, 8 vol), and the layers were stirred for 15 min. The layers were separated and the combined organic layers from the first two extractions were recharged to the kettle.

The batch was vacuum distilled (29 in Hg, <65° C.) to a final volume of 124 L (10 vol). Methanol (182 L, 15 vol) was charged to the reactor and the batch was vacuum distilled (27.5 in Hg, 16.7° C.) until the final volume was 128 L (10 vol). Methanol (182 L, 15 vol) was charged to the reactor and the batch was vacuum distilled (27.3 in Hg, 17.0° C.) until the final volume was 128 L (10 vol). The batch was adjusted to 50.5° C. and DI H₂O (182 L, 15 vol) was charged over 2 hours such that the batch temperature remained at 45-55° C. The batch was vacuum distilled (28.4 in Hg, <65° C.) to a final volume of 122 L (10 vol). DI H₂O (60.5 L, 5 vol) was charged to the batch, and the temperature was brought to 15-25° C. The batch was stirred at this temperature for 60 hours and the batch was transferred to the Rosenmund hastelloy agitated filter and was conditioned until liquid stopped eluting. To the reactor was charged DI H₂O (121 L, 10 vol) and the rinse was transferred to the solids and was conditioned until liquid stopped eluting. The product was dried under nitrogen flow at 40-70° C. for 6 days to give compound (V) (13.0 kg, 88% yield).

Specifications of obtained solid: ¹H NMR (consistent with compound (V)); appearance: light yellow solid; KF (% water): 0.02%; ¹H NMR (d₆-DMSO) weight assay versus 1,4-dimethoxybenzene (96.3%); and HPLC purity (area % @ 247 nm): 99.1%.

### Steps 4a) and 4b): Preparation of the Compound of Formula (III)

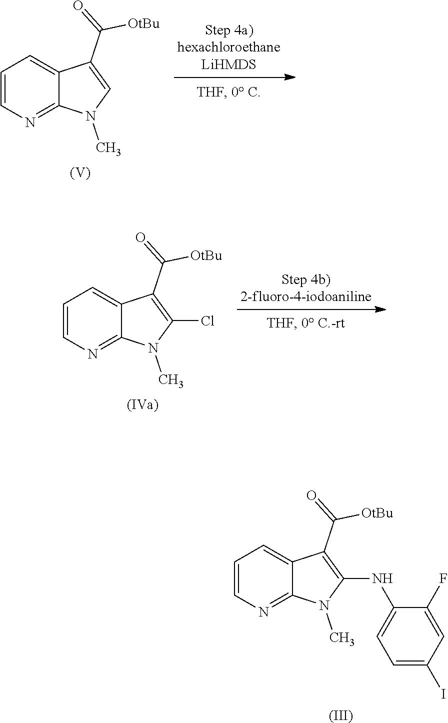

To a 400 L reactor, inerted with N₂ flow at 15 L/min for days, was charged 1 M LiHMDS (83.5 kg, 15.1 vol), stirring was started, and the batch temperature was adjusted to −5-5° C. In a separate vessel, inerted with N₂ flow at 15 L/min for days, was charged compound (V) (6.20 kg), anhydrous THF (23.1 kg (with 4.45 kg withheld to wash the kettle and lines after the transfer), 5 vol (total)), and hexachloroethane (7.27 kg), and the contents were stirred for 20 minutes to ensure all solids completely dissolved. The reaction solution was transferred to the reactor over 50 minutes to ensure the batch temperature remained at 0-10° C. (the 4.45 kg of THF withheld was used to rinse the noted solution vessel and this was also charged to the reactor during this time). After stirring for 1 h at 0-10° C., HPLC analysis revealed 100% conversion to compound (IVa).

To a separate vessel, inerted with N₂ flow at 15 L/min for 1 h, was charged 2-fluoro-4-iodoaniline (6.64 kg) and anhydrous THF (11.1 kg, 2 vol), and was stirred for 75 min to ensure all solids were completely dissolved. The 2-fluoro-4-iodoaniline solution was charged to the reactor over the course of 1 h to ensure the batch temperature remained at 0-10° C. The batch temperature was adjusted to 15-25° C. and stirred for 9.5 h. HPLC analysis revealed 1.2% remaining compound (IVa), so the batch temperature was adjusted to −5-5° C.

In a separate vessel, an ammonium chloride solution was prepared by charging DI H₂O (18.6 kg, 3 vol) and ammonium chloride (6.88 kg) and stirring the solution for 12 minutes. (Note: all solids may not fully dissolve). The ammonium chloride solution was transferred to the reactor over the course of 75 min to ensure the batch temperature remained at 5-15° C., and the batch was vacuum distilled (27.16 in Hg, max temp 35.2° C.) to a final volume of 48.5 L (8 vol). DI H₂O (75 L, 12 vol) was charged and the batch was vacuum distilled to a final volume of 94 L (16 vol). The batch temperature was adjusted to 10-20° C., EtOH (22.0 kg, 4.5 vol) was charged while maintaining the batch temperature at 10-20° C., the resulting suspension was stirred for 15 min, and the batch was filtered. The reactor was rinsed with DI H₂O (62.8 L, 10 vol), the rinse was transferred to the filter, and the solids were conditioned until liquids stopped dripping.

To the reactor was charged the filtered solids, EtOH (48.9 kg, 10 vol), and the batch was heated to 40-50° C. with stirring. The batch was held at 40-50° C. for 30 min, and was then cooled to 0-10° C. The batch was filtered to the same filter used previously, the reactor was charged with EtOH (25 kg, 5 vol), and the rinse was passed over the filtered solids. The solids were conditioned until liquid stopped dripping, transferred to a vacuum oven at 50° C., and were dried for 64 h to give product (III) (11.7 kg, 93.6%).

Specifications of obtained solid: ¹H NMR (consistent with compound (III)); appearance: brown solid; KF (% water): 0.041%; ¹H NMR (d₆-DMSO) weight assay versus 1,4-dimethoxybenzene (99.4%); and HPLC purity (area % @ 247 nm): 100%.

### Step 5): Preparation of the Compound of Formula (II)

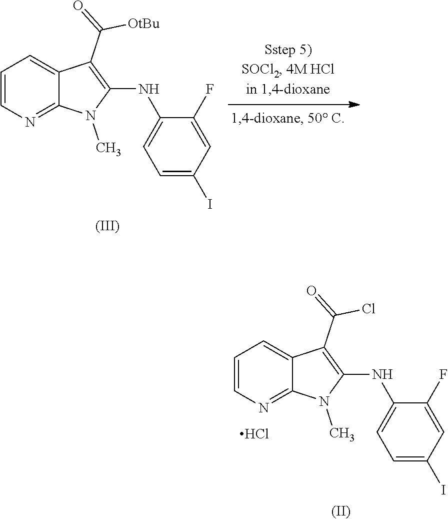 [SMILES: Cn1c(Nc2ccc(I)cc2F)c(C(=O)OC(C)(C)C)c2cccnc21 ; "(III)" ; Cn1c(Nc2ccc(I)cc2F)c(C(=O)Cl)c2cccnc21 ; Cl ; "(II)"]

To a 400 L reactor, inerted with N₂ flow at 10 L/min for 1 day with a 2 M NaOH scrubber attached, was charged compound (III) (11.7 kg), 1,4-dioxane (52.5 L, 4.5 vol), and stirring was started with the batch held between 15-25° C. While maintaining the batch temperature <30° C., thionyl chloride (29.7 kg) was charged over a 45 minute period. (Note: this addition is slightly exothermic). While maintaining the batch temperature <30° C., 4 M HCl in 1,4-dioxane (39.3 kg, 6.0 eq.) was charged over a 45 minute period. (Note: this addition is slightly exothermic). The batch was heated to 50-55° C. and held at this temperature for 17 h. HPLC analysis revealed 98% conversion of compound (III) to compound (II), so the batch temperature was adjusted to 15-25° C.

To the reactor was charged n-heptane (120 L, 10 vol, treated with 200 ppm Statsafe 6000) and the batch was vacuum distilled (28 in Hg, max temp 35.6° C.) to a final volume of 86 L (7.5 vol). To the reactor was charged n-heptane (123 L, 10 vol) and the batch was vacuum distilled (28 in Hg, max temp 24.0° C.) to a final volume of 86 L (7.5 vol). To the reactor was charged n-heptane (123 L, 10 vol) and the batch was vacuum distilled (29 in Hg, max temp 20.6° C.) to a final volume of 86 L (7.5 vol). To the reactor was charged n-heptane (116 L, 10 vol) and the batch was vacuum distilled (29 in Hg, max temp 21.0° C.) to a final volume of 80 L (7.5 vol). To the reactor was charged n-heptane (120 L, 10 vol) and the batch was vacuum distilled (28 in Hg, max temp 22.0° C.) to a final volume of 83 L (7.5 vol). The batch was filtered under N₂, the reactor was rinsed with n-heptane (55.0 L, 5 vol), and the rinse was passed over the filtered solids. The material was dried in the filter, under vacuum, with an N₂ flow of 50 L/min for 3 days to give product (II) (11.8 kg, >100%).

Specifications of obtained solid: ¹H NMR (NA); appearance: grey powder; KF (% water): 0.015%; ¹H NMR (d₆-DMSO) weight assay versus 1,4-dimethoxybenzene (NA); and HPLC purity (area % @ 247 nm): 95.9%.

Steps 6a) and 6b): Preparation of the Compound of Formula (I)

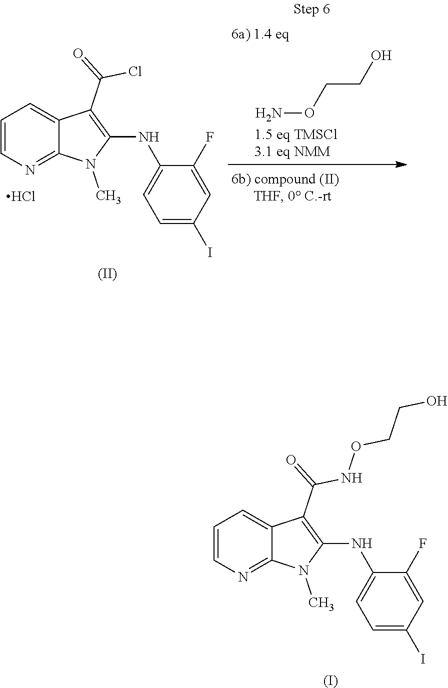 [SMILES: Cn1c(Nc2ccc(I)cc2F)c(C(=O)Cl)c2cccnc21 ; Cl ; NOCCO ; "(II)" ; Cn1c(Nc2ccc(I)cc2F)c(C(=O)NOCCO)c2cccnc21 ; "(I)"]

To a 400 L reactor, inerted with N₂ flow at 15 L/min for 26.5 h, was charged 2-(aminooxy)ethanol (2.43 kg), THF (63 L, 6 vol) and 4-methylmorpholine (7.68 L) and the batch temperature was adjusted to −5-5° C. with stirring. Chlorotrimethylsilane (4.29 L) was charged over 30 minutes such that the batch temperature remained between 0-10° C. and was stirred at this temperature for 45 min.

To a separate vessel, inerted with N₂ flow at 10 L/min for 5 h, was charged compound (II) (10.5 kg) and THF (105 L, 10 vol) and was stirred for 20 min at rt to form a homogeneous suspension. The compound (II) suspension was charged to the reactor over a 1.5 h period such that the batch temperature remained <10° C., and the batch was stirred at −5-5° C. for 30 min. HPLC analysis revealed 0.87% residual compound (II) relative to compound (I), so the batch temperature was adjusted to 15-25° C.

To a 200 L Schott reactor, inerted with N₂ flow at 20 L/min for 2 h, was charged Darco G-60 (5.25 kg). The batch was transferred to the Schott reactor and stirred with the charcoal for 45 min. This was filtered through a 0.4 m in-line filter and was transferred back to the reactor. DI H₂O (105 L, 10 vol) was charged to the reactor over a 45 min period (during which time the batch temperature rose from 13.6° C. to 24.0° C.). The batch was vacuum distilled (27 in Hg, max temp 20.7° C.) to a total of volume of 155 L (15 vol). The batch temperature was maintained at 15-25° C. and MTBE (94.5 L, 9 vol) was charged to the reactor. The batch was vacuum distilled (26 in Hg, max temp 26.6° C.) to a final volume of 133 L (13 vol). The batch temperature was maintained at 15-25° C. and MTBE (94.5 L, 9 vol) was charged to the reactor. The batch was vacuum distilled (26 in Hg, max temp 19.3° C.) to a final volume of 133 L (13 vol). The batch temperature was maintained at 15-25° C. and EtOH (94.5 L) was charged to the reactor.

The solids were filtered, the reactor was charged with DI H₂O (52.5 L, 5 vol), and the rinse was passed over the collected solids. The reactor was charged with MTBE (52.5 L, 5 vol), and the rinse was passed over the collected solids. The reactor was again charged with MTBE (52.5 L, 5 vol) and the rinse was passed over the collected. The solids were charged to the reactor, followed by DI H₂O (105 L, 10 vol), and the suspension was stirred at 15-25° C. for 40 min. The batch was filtered using the same filter setup, and the solids were conditioned until liquid stopped dripping. The solids were charged to the reactor, followed by EtOH (141 L, 13.5 vol), and the batch was heated to 70-80° C. and was stirred at this temperature for 32 min until the solids are nearly completely dissolved. To the reactor was charged DI H₂O (105 L, 10 vol) over a minimum of 2 hours such that the batch temperature remains at 70-80° C. The batch was cooled to 10-20° C. over a 13 h period and the batch was filtered into a newly setup filter. The reactor was charged four separate times with DI H₂O (52.5 L, 5 vol), and each time the rinse was passed over the collected solids as a displacement wash. The solids were conditioned until liquids stopped dripping, and were dried in a vacuum oven at 70° C. for seven days to give product (I) (5.7 kg, 53.7%).

### Example 14: Process for Preparing 2-((2-fluoro-4-iodophenyl)amino)-N-(2-hydroxyethyl)-1-methyl-1H-pyrrolo[2,3-b]pyridine-3-carboxamide (i.e., Formula (I))

The compound of formula (I) was prepared on a scale of five kilograms from the compound of formula (V) according to Steps 4a), 4b), 5), 6a), and 6b) as shown in FIG. 1.

### Steps 4a) and 4b): Preparation of the Compound of Formula (III)

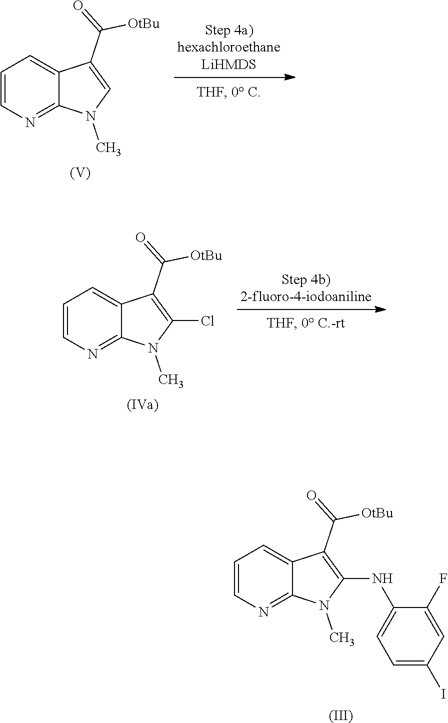

### A. Batch-1

A solution of ammonium chloride (3305 g, 61.8 moles) in purified water (9 L, 3 volumes) was prepared in a 45-L carboy.

Compound (V) (3000 g, 12.9 moles, 1 wt.), hexachloroethane (3507 g, 14.8 moles, 1.17 wt.) and anhydrous tetrahydrofuran (THF, 15 L, 5 volumes) were charged to a clean, dry 100-L jacketed glass reactor, whilst maintaining a nitrogen atmosphere. Once complete dissolution was observed the solution was transferred to a clean, dry carboy, and stored under nitrogen until required. The 100-L reactor was rinsed with anhydrous THF.

2-Fluoro-4-iodoaniline (3201 g, 13.5 moles, 1.07 wt.) and anhydrous THF (6 L, 2 volumes) were charged to a 100-L jacketed glass reactor, whilst maintaining a nitrogen atmosphere. Once complete dissolution was observed the solution was transferred to a clean, dry carboy, and stored under nitrogen until required. The 100-L reactor was rinsed with anhydrous THF.

Lithium bis(trimethylsilyl)amide (45 L, 1M in THF, 45 moles, 15 volumes) was charged to a 100-L jacketed glass reactor whilst maintaining a nitrogen atmosphere. The solution was stirred and cooled to 2° C. The previously prepared solution of compound (V)/hexachloroethane in THF was added via a peristaltic pump over 51 minutes whilst maintaining an internal temperature of <10° C. (T max was 8.3° C.). The batch was stirred for an additional 41 minutes prior to submission of a sample for in-process UPLC. Compound (V) was not detected. The previously prepared solution of 2-fluoro-4-ioaniline in THF was added via a peristaltic pump over 61 minutes whilst maintaining an internal temperature of <10° C. (T max was 8.5° C.). The batch temperature was adjusted to 20±5° C. and stirred for an additional 15 hours, 28 minutes prior to submission of a sample for in-process UPLC. Compound (IVa) was 0.69 area %. The batch was cooled to 2° C. over 36 minutes then the above prepared ammonium chloride solution was added whilst maintaining the internal temperature <10° C. (T max was 7.5° C.). The batch was distilled under vacuum (25 inches Hg), at a jacket temperature of 50° C., to 27 L (9 volumes). The distillation time was approximately 8 hours. Purified water (36 L, 12 volumes) was added and the batch distilled under vacuum (27 inches Hg), at a jacket temperature of 60° C., to 38 L (12.6 volumes). The distillation time was approximately 8.5 hours. Significant foaming of the batch was observed near the end of the distillation which resulted in material being pushed into the reactor head and riser. Ethanol (11 L, 3.7 volumes) was added. Attempts to wash down the walls of the reactor by circulation the batch were unsuccessful due to the thickness and granularity of the batch. During the attempted circulation a small crack in the BOV was noted. The batch was transferred to carboys while a new BOV was installed. Additional ethanol (5 L, 1.7 volumes) was employed to facilitate transfer of the batch and ensure minimal transfer losses. Once returned to the 100-L reactor the batch was stirred at 15° C. for 12 hours, 20 minutes then transferred to a 24 inch filter dressed with cellulose filter paper. Filtration was facile (10 minutes) and the majority of the batch was easily removed from the reactor. The reactor was rinsed with purified water (30 L, 10 volumes) and the reactor rinse used to wash the wet cake. The wet cake (crude compound (III)) was allowed to condition under nitrogen for 18 hours and 24 minutes. The wet cake was returned to the 100-L reactor using ethanol (38 L, 12.7 volumes) to aid the transfer. The batch temperature was adjusted to 20±5° C. and the contents were stirred for 41 minutes. The batch temperature was adjusted to 45±5° C. and the contents were stirred at that temperature for 2 hours and 18 minutes. The batch was cooled to 5±5° C., stirred at that temperature for 2 hours and 44 minutes then transferred to a 24 inch filter dressed with cellulose filter paper. The filter cake was washed with ethanol (20 L, 6.7 volumes) and conditioned under nitrogen for 14 hours and 19 minutes. The filter cake was dried to constant weight at 50±5° C. under vacuum to afford 5229 g of compound (III) as an off-white solid.

### B. Batch-2

A solution of ammonium chloride (3001 g, 56.1 moles, 1.1 wt.) in purified water (8 L, 3 volumes) was prepared in a 45-L carboy.

Compound (V) (2750 g, 11.8 moles, 1 wt.), hexachloroethane (3210 g, 13.6 moles, 1.17 wt.) and anhydrous tetrahydrofuran (THF, 14 L, 5 volumes) were charged to a clean, dry 100-L jacketed glass reactor, whilst maintaining a nitrogen atmosphere. Once complete dissolution was observed the solution was transferred to a clean, dry carboy, and stored under nitrogen until required. The 100-L reactor was rinsed with anhydrous THF.

2-Fluoro-4-iodoaniline (2912 g, 12.3 moles, 1.06 wt.) and anhydrous THF (6 L, 2 volumes) were charged to a 100-L jacketed glass reactor, whilst maintaining a nitrogen atmosphere. Once complete dissolution was observed the solution was transferred to a clean, dry carboy, and stored under nitrogen until required. The 100-L reactor was rinsed with anhydrous THF.

Lithium bis(trimethylsilyl)amide (41 L, 1M in THF, 41 moles, 15 volumes) was charged to a 100-L jacketed glass reactor (R100-4) whilst maintaining a nitrogen atmosphere. The solution was stirred and cooled to 1.8° C. The previously prepared solution of compound (V)/hexachloroethane in THF was added via a peristaltic pump over 55 minutes whilst maintaining an internal temperature of <10° C. (T max was 8.4° C.). The batch was stirred for an additional 32 minutes prior to submission of a sample for in-process UPLC. Compound (V) was not detected. The previously prepared solution of 2-fluoro-4-ioaniline in THF was added via a peristaltic pump over 69 minutes whilst maintaining an internal temperature of <10° C. The batch temperature was adjusted to 20±5° C. and stirred for an additional 13 hours, 50 minutes prior to submission of a sample for in-process UPLC. Compound (IVa) was 0.82 area %. The batch was cooled to 1.9° C. over 58 minutes then the above prepared ammonium chloride solution was added whilst maintaining the internal temperature <10° C. The batch was distilled under vacuum (26 inches Hg), at a jacket temperature of <65° C., to 24 L (9 volumes). The distillation time was approximately 8 hours. Purified water (33 L, 12 volumes) was added and the batch distilled under vacuum (28 inches Hg), at a jacket temperature of <65° C., to 41.5 L (15 volumes). The distillation time was approximately 5 hours. Significant foaming of the batch was observed near the end of the distillation (reference ALB-DEV-18-0135). Ethanol (12 L, 4 volumes) was added to the 100-L reactor and the batch was stirred at 15±5° C. for 2 hours, 31 minutes. The batch was then transferred to a 24 inch filter dressed with cellulose filter paper. The reactor was rinsed with purified water (28 L, 10 volumes) and the reactor rinse used to wash the wet cake. The wet cake (crude compound (III)) was allowed to condition under nitrogen for 67 hours and 25 minutes. The wet cake was returned to the 100-L reactor using ethanol (35 L, 12.7 volumes) to aid the transfer. The batch temperature was adjusted to 20±5° C. and the contents were stirred for 35 minutes. The batch temperature was adjusted to 45±5° C. and the contents were stirred at that temperature for 44 minutes. The batch was cooled to 5±5° C., stirred at that temperature for 16 hours and 36 minutes then transferred to a 24 inch filter dressed with cellulose filter paper. The filter cake was washed with ethanol (18 L, 6.5 volumes) and conditioned under nitrogen for 1 hour and 7 minutes. The filter cake was dried to constant weight at 50±5° C. under vacuum to afford 4776 g compound (III) as an off-white solid.

### C. In-Process Test Results for Steps 4a) and 4b)

| | Test | | | |
|---|---|---|---|---|
| Test | Method | Specification | Batch-1 | Batch-2 |
| UPLC | TM-4a | Compound (V) ≤1 area % | Not | Not |
| | | by conversion with respect | Detected | Detected |
| | | to Compound (IVa) | | |
| UPLC | TM-4b | Compound (IVa) ≤5 area | 0.69% | 0.82% |
| | | % by conversion with | | |
| | | respect to Compound (III) | | |

### Step 5): Preparation of the Compound of Formula (II)

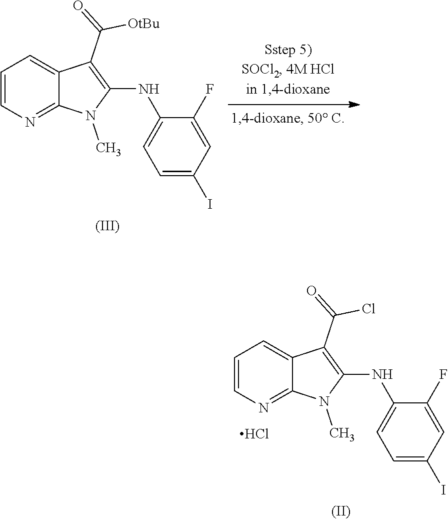 [SMILES: Cn1c(Nc2ccc(I)cc2F)c(C(=O)OC(C)(C)C)c2cccnc21 ; "(III)" ; Cn1c(Nc2ccc(I)cc2F)c(C(=O)Cl)c2cccnc21 ; Cl ; "(II)"]

### A. Batch-1

A 100-L drop bottom glass jacketed reactor was equipped with a two channel chart recorder, a thermal control unit and a condenser. The reactor was vented via a scrubber system filled with 4M sodium hydroxide solution. A nitrogen bleed was applied then the reactor was charged with anhydrous 1,4-dioxane (24.5 L, 4.6 volumes) and compound (III) (5209 g, 11.1 moles, 1 wt., Batch-1). The batch temperature was adjusted to 20±5° C. Thionyl chloride (8.1 L, 1.6 volumes) was charged to the reactor while maintaining the batch temperature <30° C. then 4 molar hydrogen chloride in 1,4-dioxane (16.7 L, 3.2 volumes) was charged to the reactor while maintaining the batch temperature <30° C. The batch temperature was heated to 55±5° C. and the contents stirred 14 hours and 29 minutes. After this time, the batch was sampled for UPLC analysis to give 2.6% compound (III). The batch temperature was adjusted to 20±5° C. then n-heptane (24 L, 4.6 volumes) was charged to the batch. The batch was distilled to 43 L (8 volumes) while maintaining the jacket temperature <50° C. and maintaining the batch temperature <40° C. n-Heptane (36 L, 7 volumes) was charged to the reactor and the contents distilled under the same conditions to 32 L (6 volumes). n-Heptane (47 L, 9 volumes) was charged to the reactor and the contents distilled under the same conditions to 33 L (6 volumes) a total of three times. The batch temperature was adjusted to 20±5° C. and the batch was stirred for 17 hours and 33 minutes. The batch was filtered over polypropylene cloth and cellulose filter paper. The filter cake was washed with n-heptane (52 L, 10 volumes), conditioned under nitrogen for 48 hours and 43 minutes then dried to constant weight at 30±5° C. under vacuum to afford 5211 g compound (II).

### B. Batch-2

A 100-L drop bottom glass jacketed reactor was equipped with a two channel chart recorder, a thermal control unit and a condenser. The reactor was vented via a scrubber system filled with 4M sodium hydroxide solution. A nitrogen bleed was applied then the reactor was charged with anhydrous 1,4-dioxane (22 L, 4.6 volumes) and compound (III) (4747 g, 10.2 moles, 1 wt., Batch-2). The batch temperature was adjusted to 20±5° C. Thionyl chloride (7.4 L, 1.6 volumes) was charged to the reactor while maintaining the batch temperature <30° C. then 4 molar hydrogen chloride in 1,4-dioxane (15.3 L, 3.2 volumes) was charged to the reactor while maintaining the batch temperature <30° C. The batch temperature was heated to 55±5° C. and the contents stirred 17 hours and 44 minutes. After this time, the batch was sampled for UPLC analysis to give 2.0% compound (III). The batch temperature was adjusted to 20±5° C. then n-heptane (22 L, 4.6 volumes) was charged to the batch. The batch was distilled to 39 L (8 volumes) while maintaining the jacket temperature <50° C. and maintaining the batch temperature <40° C. n-Heptane (33 L, 7 volumes) was charged to the reactor and the contents distilled under the same conditions to 30 L (6 volumes). n-Heptane (43 L, 9 volumes) was charged to the reactor and the contents distilled under the same conditions to 30 L (6 volumes) a total of three times. The batch temperature was adjusted to 20±5° C. and the batch was stirred for 2 hours and 4 minutes. The batch was filtered over polypropylene cloth and cellulose filter paper. The filter cake was washed with n-heptane (48 L, 10 volumes), conditioned under nitrogen for 62 hours and 22 minutes then dried to constant weight at 30±5° C. under vacuum to afford 4711 g compound (II).

### C. In-Process Test Results for Step 5

| | Test | | | |
|---|---|---|---|---|
| Test | Method | Specification | Batch-1 | Batch-2 |
| UPLC | TM-5 | Compound (III) ≤8.0% | 2.6% | 2.0% |
| | | (by conversion | | |
| | | based on the ratio of | | |
| | | (III) to (II), Area %, | | |
| | | area of (III)/(area of | | |
| | | (III) + (II)) | | |

### Steps 6a) and 6b): Preparation of the Compound of Formula (I)

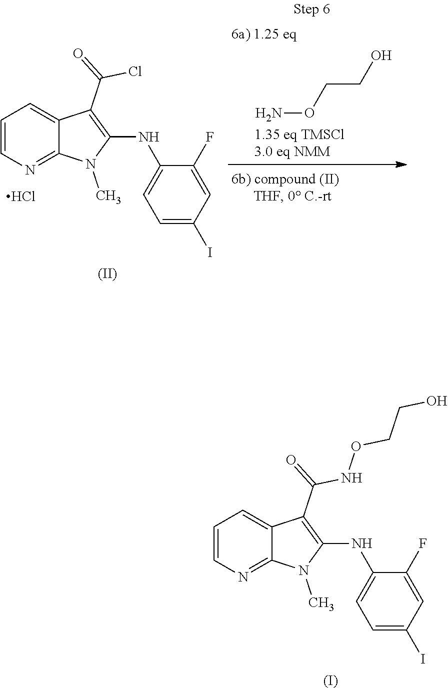 [SMILES: Cn1c(Nc2ccc(I)cc2F)c(C(=O)Cl)c2cccnc21 ; Cl ; NOCCO ; "(II)" ; Cn1c(Nc2ccc(I)cc2F)c(C(=O)NOCCO)c2cccnc21 ; "(I)"]

The 200-L Hastelloy reactor was flushed with nitrogen then charged with THF (38.5 L, 4 volumes), 2-(aminooxy)ethanol (2619 g, 65.5 wt/wt, 22.3 moles) and n-methylmorpholine (6.9 L, 62.8 mol, 0.66 wt.). The batch was cooled to 0±5° C. and chlorotrimethylsilane (3914 g, 36.0 moles, 0.41 wt.) was charged to the reactor while maintaining the batch temperature <10° C. The batch was stirred at 0±5° C. for 1 hour.

A 72-L glass reactor was equipped with a chart recorder and charged with THF (32 L, 3.33 volumes) and compound (II) (3200 g, 6.9 moles, 0.33 wt.). The contents were stirred 5 minutes until a fine suspension was observed. The suspension was transferred to the 200-L Hastelloy reactor over 78 minutes while maintaining the batch temperature <10° C. A second portion of THF (32 L, 3.33 volumes) and compound (II) (3201 g, 6.9 moles, 0.33 wt.) was charged to the 72-L reactor. The contents were stirred 3 minutes until a fine suspension was observed. The suspension was transferred to the 200-L Hastelloy reactor over 21 minutes while maintaining the batch temperature ≤10° C. A third portion of THF (32 L, 3.33 volumes) and compound (II) (3172 g, 6.8 moles, 0.33 wt.) was charged to the 72-L reactor. The contents were stirred 6 minutes until a fine suspension was observed. The suspension was transferred to the 200-L Hastelloy reactor over 22 minutes while maintaining the batch temperature <10° C.

The temperature of the batch in the 200-L Hastelloy reactor was adjusted to 0±5° C. and the contents stirred 33 minutes. After this time, the batch was sampled for UPLC analysis to give 4.5% compound (II). The batch was stirred at 0±5° C. for an additional 1 hour and 47 minutes before sampling a second time for UPLC analysis to give 3.7% compound (II). The batch temperature was adjusted to 20±5° C. Approximately ⅓ of the batch was transferred from the 200-L Hastelloy reactor to the 72-L reactor, treated with activated charcoal (1.6 kg, 0.17 wt) and stirred for at least 30 minutes. The batch was filtered over a Celite pad and the batch solution was held in carboys at room temperature until required for further processing. This operation was repeated an additional two times with the remainder of the batch. The 200-L Hastelloy reactor was cleaned and rinsed with THF (20 L). The carbon treated batch solution was returned to the 200-L Hastelloy reactor via a transfer line fitted with an inline filter. The carboys were rinsed with THF (20 L, 2 volumes) and the rinse was transferred to the 200-L Hastelloy reactor via the transfer line fitted with the inline filter. Approximately half of the batch was transferred to clean dry glass carboys and held at room temperature. Purified water (38 L, 4 volumes) was charged to the 200-L Hastelloy reactor while maintaining the batch temperature <30° C. The batch was stirred for 8 minutes then transferred to clean dry glass carboys and held at 2-8° C. overnight. The other half of the batch was returned to the 200-L Hastelloy reactor and treated with purified water (38 L, 4 volumes) while maintaining the batch temperature <30° C. The batch was stirred overnight at 2-8° C. The batch was concentrated under reduced pressure with a jacket temperature of <45° C. to a final batch volume of 104 L (11 volumes). Methyl tert-butyl ether (MTBE, 58 L, 6 volumes) was transferred to the 200-L Hastelloy reactor using an inline filter and the contents of the reactor were concentrated under reduced pressure with a jacket temperature of 50° C. to a final batch volume of 103-106 L (11 volumes) a total of four times. Pre-filtered MTBE (58 L, 6 volumes) was charged to the 200-L Hastelloy reactor, the batch temperature was adjusted to 20±5° C. and the batch was stirred 17 hours and 13 minutes. The batch was filtered over polypropylene cloth and cellulose filter paper. The filter cake was washed with pre-filtered MTBE (2×48 L, 2×5 volumes) then conditioned under nitrogen for 63 hours and 18 minutes. The 200-L Hastelloy reactor was cleaned and rinsed with pre-filtered MTBE (23 L) before returning the filter cake with purified water (96 L, 10 volumes). The batch temperature was adjusted to 20±5° C. and the batch was stirred 57 minutes. The batch was filtered over polypropylene cloth and cellulose filter paper. The filter cake was washed with purified water (48 L, 5 volumes) then conditioned under nitrogen for 16 hours and 58 minutes.

The wet cake was returned to the reactor with pre-filtered ethanol (110 L, 11 volumes). The batch temperature was adjusted to 80±5° C. and the batch was stirred until complete dissolution was observed (note: dissolution was observed at 75° C.). Purified water (82 L, 8.5 volumes) was charged to the reactor over 1 hour and 8 minutes while maintaining the batch temperature at >70° C. The batch temperature was slowly adjusted to 15±5° C. over approximately 16 hours and the batch stirred at 15±5° C. for approximately 6 hours. The batch was filtered over polypropylene cloth and cellulose filter paper. The filter cake was washed with purified water (4×48 L, 4×5 volumes), conditioned under nitrogen for 18 hours and 48 minutes then dried to constant weight at 65±5° C. under vacuum to afford 4605 g compound (I).

After this time, the batch was sampled for Karl Fisher analysis (USP<921>) to give 1.3% water; OVI analysis to give 950 ppm ethanol; and none detected for THF, MTBE, 1,4-dioxane, and n-heptane. The batch was dried an additional 43 hours and 35 minutes then sampled a second time for Karl Fisher analysis (USP<921>) to give 1.4% water. The batch was off loaded to afford 4598 g compound (I).

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A process for preparing a compound represented by formula (I): 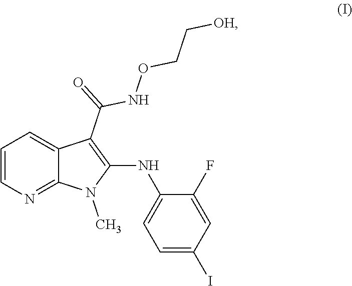 [SMILES: Cn1c(Nc2ccc(I)cc2F)c(C(=O)NOCCO)c2cccnc21]
or a salt thereof, comprising:
6a) contacting a compound represented by formula (K): 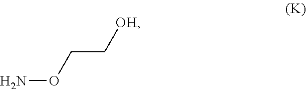
or a salt thereof, with a first base and a silylating agent in a first solvent to form a first mixture comprising an O-silyl protected compound of formula (K); and
6b) adding a second mixture comprising a compound represented by formula (II): 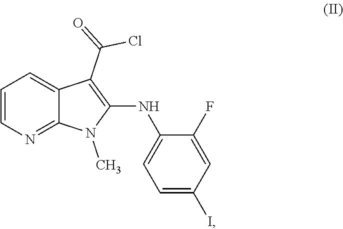 [SMILES: Cn1c(Nc2ccc(I)cc2F)c(C(=O)Cl)c2cccnc21]
or a salt therefore, to the first mixture of step 6a) to form the compound represented by formula (I).

2. The process of claim 1, wherein the compound of formula (K) is a p-toluenesulfonic acid salt thereof represented by formula (K-1):

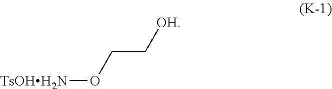

3. The process of claim 1, wherein, prior to contacting the silylating agent, the compound of formula (K) or (K-1) is first contacted with the first base in the first solvent.

4. The process of claim 1, wherein the silylating agent is trimethylsilyl chloride (TMSCl).

5. The process of claim 1, wherein the first base is a tertiary amine.

6. The process of claim 5, wherein the tertiary amine is 4-methylmorpholine.

7. The process of claim 1, wherein the first solvent comprises tetrahydrofuran (THF).

8. The process of claim 6, wherein a precipitate comprising a p-toluenesulfonic acid salt of 4-methylmorpholine is filtered prior to contacting the silylating agent.

9. The process of claim 1, wherein:
the compound of formula (K) or the salt thereof is present in an amount of from about 1.1 to about 1.5 equivalents relative to the compound of formula (II);
trimethylsilyl chloride (TMSCl) is present in an amount of from about 1.2 to about 2.0 equivalents relative to the compound of formula (II); and
4-methylmorpholine present in an amount of from about 3 to about 5 equivalents relative to the compound of formula (II), when the compound of formula (K) is in a neutral form; or 4-methylmorpholine is present in an amount of from about 4 to about 6 equivalents relative to the compound of formula (II), when the compound of formula (K) is in a salt form.

10. The process of claim 1, wherein the first mixture is formed in-situ.

11. The process of claim 1, wherein the second mixture further comprises a second solvent selected from the group consisting of tetrahydrofuran (THF), 2-methyltetrahydrofuran (2-MeTHF), acetonitrile (ACN), dichloromethane (DCM), methyl tert-butyl ether (MTBE), heptanes, isopropyl acetate (IPAc), or combinations thereof.

12. The process of claim 11, wherein the second solvent comprises tetrahydrofuran (THF).

13. The process of claim 1, wherein the second mixture is a slurry comprising a HCl salt of formula (II).

14-15. (canceled)

16. The process of claim 1, further comprising prior to step 6a):
5) contacting a compound represented by formula (III): 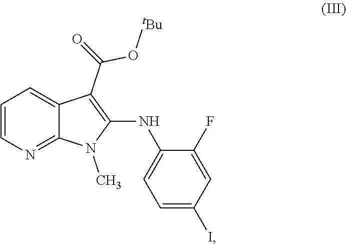
or a salt thereof, with a first chlorinating agent and hydrogen chloride in a third solvent to form a HCl salt of the compound represented by formula (II):

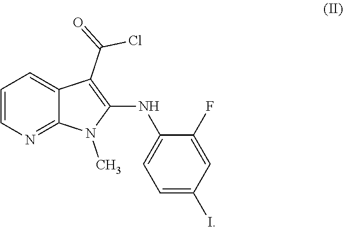 [SMILES: Cn1c(Nc2ccc(I)cc2F)c(C(=O)Cl)c2cccnc21]

17. The process of claim 16, wherein the first chlorinating agent is thionyl chloride.

18. The process of claim 16, wherein the first chlorinating agent is present in an excess amount of at least 5 equivalents relative to the compound of formula (III).

19. (canceled)

20. The process of claim 16, wherein hydrogen chloride is present in an amount of from about 5 to about 6 equivalents relative to the compound of formula (III).

21-23. (canceled)

24. The process of claim 16, further comprising prior to step 5):
4a) contacting a compound represented by formula (V): 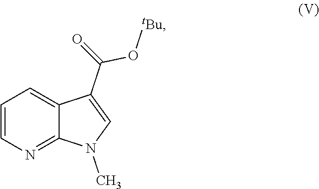
or a salt thereof, with a second chlorinating agent and a second base in a fourth solvent to form a compound represented by formula (IVa): 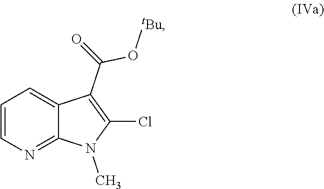
or a salt thereof, and
4b) reacting the compound of formula (IVa), or the salt thereof, with an aniline represented by formula (L): 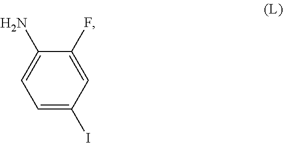
or a salt thereof, with a third base in a fifth solvent to form the compound represented by formula (III): 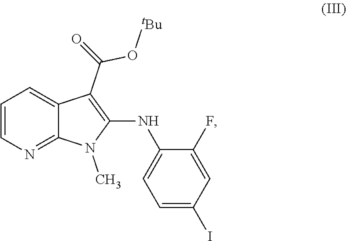
or the salt thereof.

25. The process of claim 24, wherein, in step 4a), the second chlorinating agent is hexachloroethane.

26. The process of claim 25, wherein hexachloroethane is present in an amount of from about 1.1 to about 1.5 equivalents relative to the compound of formula (V).

27. The process of claim 24, wherein the second and third bases are each independently a metal amide selected from the group consisting of lithium diisopropylamide (LDA), lithium bis(trimethylsilyl)amide (LiHMDS), potassium bis(trimethylsilyl)amide (KHMDS), and lithium 2,2,6,6,-tetramethylpiperidide (LiTMP); or
the second base is a metal amide selected from the group consisting of lithium diisopropylamide (LDA), lithium bis(trimethylsilyl)amide (LiHMDS), potassium bis(trimethylsilyl)amide (KHMDS), and lithium 2,2,6,6,-tetramethylpiperidide (LiTMP); and the third base comprises an alkali tert-butoxide selected from the group consisting of sodium tert-butoxide and potassium tert-butoxide.

28. (canceled)

29. The process of claim 27, wherein the second and third bases are each lithium bis(trimethylsilyl)amide (LiHMDS); or the second base is lithium bis(trimethylsilyl)amide (LiHMDS) and the third base comprises potassium tert-butoxide.

30. The process of claim 24, wherein, when the second and third bases are the same, steps 4a) and 4b) are conducted in one-pot.

31. (canceled)

32. The process of claim 24, wherein, in step 4b), the aniline of formula (L) is present in an amount of no more than 1.1 equivalent relative to the compound of formula (IVf): and/or the aniline of formula (L) is added to a reaction mixture of step 4a) comprising the compound of formula (IVa), or the salt thereof.

33. (canceled)

34. The process of claim 24, wherein the fourth and fifth solvents each comprise tetrahydrofuran (THF).

35-39. (canceled)

40. A process for preparing a compound represented by formula (K): 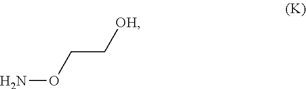
or a salt thereof, comprising:
7) contacting 2-hydroxyisoindoline-1,3-dione represented by the formula: 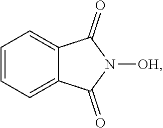 [SMILES: O=C1c2ccccc2C(=O)N1O]
with 2-bromoethanol and a non-nucleophilic base in an aprotic solvent to form 2-(2-hydroxyethoxy)isoindoline-1,3-dione represented by formula (J): 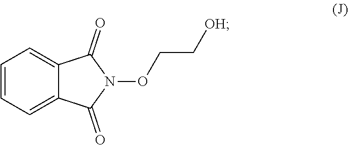 [SMILES: O=C1c2ccccc2C(=O)N1OCCO]
8a) treating 2-(2-hydroxyethoxy)isoindoline-1,3-dione with ammonia in an alcohol solvent to provide the compound of formula (K); and
8b) optionally converting the compound of formula (K) to the salt thereof.

41. The process of claim 40, wherein the non-nucleophilic base is a tertiary amine selected from the group consisting of triethyl amine and 3 N,N-diisopropylethylamine; and the aprotic solvent is acetonitrile, or
the non-nucleophilic base is 1,8-diazabicyclo[5.4.0]undec-7-ene (DBU): and the aprotic solvent is dimethylformamide (DMF).

42. (canceled)

43. The process of any one of claim 40, wherein, in step 8a), the alcohol solvent is methanol: and/or ammonia is a solution in methanol at a concentration of from about 3.5 M to about 7 M.

44. (canceled)

45. The process of any one of claim 40, wherein, in step 8b), the salt of formula (K) is a p-toluenesulfonic acid salt represented by formula (K-1):

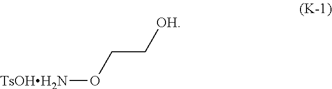

46-51. (canceled)

52. A compound represented by formula (X):

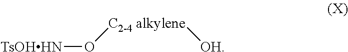

53. The compound of claim 52, represented by formula (K-1): [chemical structure]
